(19) United States
(12) Patent Application Publication
(10) Pub. No.: US 2022/0362275 A1
(43) Pub. Date: Nov. 17, 2022

(54) Method and Compositions for Treating Glioblastoma

(71) Applicant: PHOENIX BIOTECHNOLOGY, INC., San Antonio, TX (US)

(72) Inventors: ROBERT A. NEWMAN, SURRY, ME (US); OTIS C. ADDINGTON, SAN ANTONIO, TX (US); PEIYING YANG, Sugarland, TX (US); CLAUDIO FESTUCCIA, L'Aquila (IT); JOSE R MATOS, PLANO, TX (US)

(73) Assignee: PHOENIX BIOTECHNOLOGY, INC., San Antonio, TX (US)

(21) Appl. No.: 17/830,957

(22) Filed: Jun. 2, 2022

Related U.S. Application Data
(63) Continuation-in-part of application No. PCT/US2020/059784, filed on Nov. 10, 2020.
(60) Provisional application No. 63/028,767, filed on May 22, 2020.
(60) Provisional application No. 62/942,337, filed on Dec. 2, 2019.

(51) Int. Cl. A61K 31/7048; A61K 31/19; A61K 31/495; A61P 35/00; A61N 5/10

(52) U.S. Cl. CPC A61K 31/7048; A61K 31/19; A61K 31/495; A61P 35/00; A61N 5/10

(57) ABSTRACT

A method of treating glioma is provided. A combination protocol as described herein includes radiotherapy, chemotherapy, and pharmacotherapy. Resection of the glioma is optionally included in the combination protocol. Pharmacotherapy is conducted with an oleandrin-containing composition. Treatment of glioblastoma is particularly contemplated.

### CROSS-REFERENCE TO EARLIER-FILED APPLICATIONS

The present application claims the benefit of and is a continuation-in-part of application No. PCT/US2020/059784, filed Nov. 10, 2020, which claims the benefit of provisional applications No. 62/942,337, filed Dec. 2, 2019, and No. 63/028,767, filed May 22, 2020, the entire disclosures of which are hereby incorporated by reference.

### FIELD OF THE INVENTION

The present invention concerns a method of treating glioblastoma by administration of a pharmaceutical composition, which may comprise oleandrin, an extract comprising oleandrin, an extract of Nerium species, one or more components of an extract of Nerium species, or a mixture of triterpenes. The composition optionally further comprises one or more other active ingredients. Some embodiments concern treatment of GM (glioma) using a combination protocol comprising radiotherapy, chemotherapy, and pharmacotherapy (administration of a pharmaceutical composition as described herein). The combination protocol optionally further comprises resection of the GM tumor. The GM can be any of Grade I, Grade II, Grade III or Grade IV. The GM can be newly diagnosed GM, recurrent GM, or treatment resistant GM.

### BACKGROUND OF THE INVENTION

In the United States, about 17,000 people a year are diagnosed with cancer that began in or next to the brain. These are called primary brain cancers. Another 100,000 people are diagnosed with cancer in the brain or spinal cord that spread from another place in the body. These are called secondary brain cancers.

Gliomas are a class of primary brain tumors. They are some of the fastest-growing brain tumors. The different types of gliomas include astrocytoma (which include low-grade astrocytomas (Grade I pilocytic astrocytoma, and Grade II difuse astrocytoma), anaplastic astrocytoma (Grade III), glioblastoma (Grade IV, GBM, also known as glioblastoma multiforme)), ependymoma, and oligodendroglioma. GBM is thus considered a subset of GM.

GBM is the most common malignant (cancerous) adult brain tumor and one of the fastest-growing tumors of the central nervous system. 240,000 people die world-wide each year from all brain cancers—with GBM being the most common, aggressive and lethal form of primary brain cancer. The median age of diagnosis is 58 y, and the median age at death is 65 y. Key risk factors include the following: male, adults age 45-65, with familial history. 18,000 people are diagnosed annually in US, alone, with GBM, with 13,000 deaths each year. Less than 3% of those diagnosed with GBM survive five years, and a majority of patients don't survive more than a year after diagnosis.

After initial surgical resection of a glioma, the most common course of treatment for GBM includes radiotherapy and/or chemotherapy.

Mann et al. (“Advances in Radiotherapy for Glioblastoma” in Front. Neurol. (January 2018), 8, 1-11, article 748; doi: 10.3389/fneur.2017.00748) discloses external beam radiotherapy (RT) treatment protocols for GBM.

Fernandes et al. (“Current Standards of Care in Glioblastoma Therapy” in Glioblastoma. Steven De Vleeschouwer (Editor), Codon Publications, Brisbane, Australia, ISBN: 978-0-9944381-2-6; Doi: dx.doi.org/10.15586/codon.glioblastoma. 2017, pp 197-241), mention that the standard of care for GBM includes surgical resection of the tumor followed by radiotherapy (RT) plus concomitant and maintenance temozolomide (TMZ) chemotherapy; even so, almost all patients experience tumor progression with nearly universal mortality. The median survival from initial diagnosis is less than 15 months, with a 2-year survival rate of 26-33%. The addition of bevacizumab to standard treatment revealed no increase in overall survival (OS) but improved progression-free survival (PFS). A multicenter retrospective study, including 503 patients with rGBM submitted to reoperation, concluded that preoperative and postoperative KPS, EOR of first re-resection, and chemotherapy after first re-resection significantly influenced survival after reoperation. Importantly, this study reported a rate of permanent new deficits after first re-resection of 8%. Fernandes et al. conclude, “Currently, no standard of care is established for recurrent or progressive GBM (rGBM).” Weller et al. (“Standards of care for treatment of recurrent glioblastoma—are we there yet?” in Neuro-Onc. (2013), 15(1), 4-47; doi: 10.1093/neuonc/nos273) disclose much of the same treatment protocols as those disclosed by Fernandes et al., and they conclude, “Despite some minor improvements in PFS, no obvious increase in survival has been associated with any particular regimen.”

The current standard of care for patients with nGBM (newly diagnosed glioblastoma) is maximum safe surgical resection followed by concurrent TMZ (temozolomide, 75 mg/m²/day for 6 weeks) and RT (60 Gy in 30 fractions) and then six maintenance cycles of TMZ (150-200 mg/m²/day for the first 5 days of a 28-day cycle sdTMZ), according to the results of the phase III EORTC 26981. For patients aged under 70 years with good PS (KPS≥60), the optimal dose fractionation schedule for external beam RT, following resection or biopsy, is 60 Gy in 2 Gy fractions delivered over 6 weeks. The QUANTEC authors emphasize that for most brain tumors, there is no clinical indication to give fractionated RT>60 Gy. In younger patients with good PS, focal reirradiation (stereotactic radiosurgery, SRS; hypofractionated stereotactic radiotherapy, HFSRT) for rGBM may improve outcomes compared to supportive care or systemic therapy alone.

TMZ is an oral chemotherapeutic drug that induces DNA methylation and tumor cytotoxicity through cell cycle arrest. The cytotoxic activity of TMZ and other alkylating agents is apparent by the formation of O⁶-methylguanine DNA adducts, which are repaired by the enzyme MGMT. Consequently, the primary mechanism of resistance to TMZ is dependent on the MGMT activity. TMZ exhibits linear pharmacokinetics with excellent bioavailability, readily enters the cerebrospinal fluid, and does not require hepatic metabolism for activation. TMZ-containing capsules are currently marketed in various dosage strengths (5 mg, 20 mg, 100 mg, 140 mg, 180 mg and 250 mg under the tradename TEMODAR® (Merck & Co., Inc., Whitehouse Station, N.J. 08889, USA; NDA 021029, the entire disclosure of which is hereby incorporated by reference). TMZ capsules are preferably administered on an empty stomach. Bedtime administration may be advised.

Dose-dense schedules of TMZ (ddTMZ) have been designed to deplete tumor MGMT levels and thereby improve activity of TMZ, particularly in the MGMT unmethylated GBM cohort. In the RTOG 0525 phase III trial, 833 patients were randomized to receive sdTMZ or ddTMZ (75-100 mg/m² days 1 through 21 of a 28-day cycle), for 6-12 cycles, after completion of concomitant RT-TMZ. The median OS (16.6 vs. 14.9 months; P=0.63) and the median PFS (5.5 vs. 6.7 months; P=0.06) were not significantly different between the two treatment arms. There was increased grade ≥3 toxicity in ddTMZ arm (34% vs. 53%; P<0.001), as well as a greater deterioration on function subscales and QoL.

GBM is well known to recur after completion of radiotherapy and/or chemotherapy. Such GBM is referred to herein as “treatment resistant GBM” or “recurrent GBM”.

Nerium oleander, a member of the Nerium species, is an ornamental plant widely distributed in subtropical Asia, the southwestern United States, and the Mediterranean. Its medical and toxicological properties have long been recognized. It has been proposed for use, for example, in the treatment of hemorrhoids, ulcers, leprosy, snake bites, cancers, tumors, neurological disorders, cell-proliferative diseases.

Extraction of components from plants of Nerium species has traditionally been carried out using boiling water, cold water, or organic solvent.

ANVIRZEL™ (U.S. Pat. No. 5,135,745 to Ozel) contains the concentrated form or powdered form of the hot-water extract of Nerium oleander. Muller et al. (Pharmazie. (1991) September 46(9), 657-663) disclose the results regarding the analysis of a water extract of Nerium oleander. They report that the polysaccharide present is primarily galacturonic acid. Other saccharides include rhamnose, arabinose and galactose. Polysaccharide content and individual sugar composition of polysaccharides within the hot water extract of Nerium oleander have also been reported by Newman et al. (J. Herbal Pharmacotherapy, (2001) vol 1, pp. 1-16). Compositional analysis of ANVIRZEL™, the hot water extract, was described by Newman et al. (Anal. Chem. (2000), 72(15), 3547-3552). U.S. Pat. No. 5,869,060 to Selvaraj et al. pertains to extracts ofNerium species and methods of production. To prepare the extract, plant material is placed in water and boiled. The crude extract is then separated from the plant matter and sterilized by filtration. The resultant extract can then be lyophilized to produce a powder. U.S. Pat. No. 6,565,897 (U.S. Pregrant Publication No. 20020114852 and PCT International Publication No. WO 2000/016793 to Selvaraj et al.) discloses a hot-water extraction process for the preparation of a substantially sterile extract.

Erdemoglu et al. (J. Ethnopharmacol. (2003) November 89(1), 123-129) discloses results for the comparison of aqueous and ethanolic extracts of plants, including Nerium oleander, based upon their anti-nociceptive and anti-inflammatory activities.

Organic solvent extracts of Nerium oleander are also disclosed by Adome et al. (Afr. Health Sci. (2003) August 3(2), 77-86; ethanolic extract), el-Shazly et al. (J. Egypt Soc. Parasitol. (1996), August 26(2), 461-473; ethanolic extract), Begum et al. (Phytochemistry (1999) February 50(3), 435-438; methanolic extract), Zia et al. (J. Ethnolpharmacol. (1995) November 49(1), 33-39; methanolic extract), and Vlasenko et al. (Farmatsiia. (1972) September-October 21(5), 46-47; alcoholic extract).

A supercritical fluid extract of Nerium species is known (U.S. Pat. Nos. 8,394,434, 8,187,644, 7,402,325) and has demonstrated efficacy in treating neurological disorders (U.S. Pat. Nos. 8,481,086, 9,220,778, 9,358,293, US 20160243143A1) and cell-proliferative disorders (U.S. Pat. No. 8,367,363). PBI-05204 is a specifically formulated botanical drug consisting of a modified supercritical CO₂ extract of Nerium oleander that has undergone both Phase I and Phase II clinical trials in the United States for treatment of patients with a variety of advanced cancers; however, it has been found to be ineffective in treating some cancers (U.S. Pat. No. 8,367,363).

Oleandrin has been suggested for the treatment of glioma. Garofalo et al. (“The Glycoside Oleandrin Reduces Glioma Growth with Direct and Indirect Effects on Tumor Cells” in J. Neurosci. (April 2017), 37(14), 3926-3939) suggest coadministration of oleandrin and temozolomide for treating glioma, neo-GBM. Teng et al. (“Systemic Anticancer Neural Stem Cells in Combination with a Cardiac Glycoside for Glioblastoma Therapy” in Stem Cells (August 2014), 32(8), 2021-2031; doi:10.1002/stem.1727) suggest coadministration of lanatoside C with a genetically engineered neural stem cell line that synthesizes and secretes TRAIL and the Gaussia luciferase blood reporter. Newman et al. (“Enhancement of radiotherapy by oleandrin is a caspase-3 dependent process” in Cancer Letters (2002), 185, 145-151) suggest that the combined use of X-ray therapy and a hot-water extract of Nerium sp. for treating PC-3 human prostate cells.

Triterpenes are known to possess a wide variety of therapeutic activities. Some of the known triterpenes include oleanolic acid, ursolic acid, betulinic acid, bardoxolone, maslinic acid, and others. The therapeutic activity of the triterpenes has primarily been evaluated individually rather than as combinations of triterpenes.

Oleanolic acid is in a class of triterpenoids typified by compounds such as bardoxolone which have been shown to be potent activators of the innate cellular phase 2 detoxifying pathway, in which activation of the transcription factor Nrf2 leads to transcriptional increases in programs of downstream antioxidant genes containing the antioxidant transcriptional response element (ARE). Bardoxolone itself has been extensively investigated in clinical trials in inflammatory conditions; however, a Phase 3 clinical trial in chronic kidney disease was terminated due to adverse events that may have been related to known cellular toxicities of certain triterpenoids including bardoxolone at elevated concentrations.

Compositions containing triterpenes in combination with other therapeutic components are found as plant extracts. Fumiko et al. (Biol. Pharm. Bull (2002), 25(11), 1485-1487) discloses the evaluation of a methanolic extract of Rosmarimus officinalis L. for treating trypanosomiasis. Addington et al. (U.S. Pat. Nos. 8,481,086, 9,220,778, 9,358,293, US 20160243143 A1) disclose a supercritical fluid extract (SCF; PBI-05204) of Nerium oleander containing oleandrin and triterpenes for the treatment of neurological conditions. Addington et al. (U.S. Pat. No. 9,011,937, US 20150283191 A1) disclose a triterpene-containing fraction (PBI-04711) of the SCF extract of Nerium oleander containing oleandrin and triterpenes for the treatment of neurological conditions. Jäger et al. (Molecules (2009), 14, 2016-2031) disclose various plant extracts containing mixtures of oleanolic acid, ursolic acid, betulinic acid and other components. Mishra et al. (PLoS One 2016 25; 11(7):e0159430. Epub 2016 Jul. 25) disclose an extract of Betula utilis bark containing a mixture of oleanolic acid, ursolic acid, betulinic acid and other components. Wang et al. (Molecules (2016), 21, 139) disclose an extract of Alstonia scholaris containing a mixture of oleanolic acid, ursolic acid, betulinic acid and other components. L. e Silva et al. (Molecules (2012), 17, 12197) disclose an extract of Eriope blanchetti containing a mixture of oleanolic acid, ursolic acid, betulinic acid and other components. Rui et al. (Int. J. Mol. Sci. (2012), 13, 7648-7662) disclose an extract of Eucaplyptus globulus containing a mixture of oleanolic acid, ursolic acid, betulinic acid and other components. Ayatollahi et al. (Iran. J. Pharm. Res. (2011), 10(2), 287-294) disclose an extract of Euphorbia microsciadia containing a mixture of oleanolic acid, ursolic acid, betulinic acid and other components. Wu et al. (Molecules (2011), 16, 1-15) disclose an extract of Ligustrum species containing a mixture of oleanolic acid, ursolic acid, betulinic acid and other components. Lee et al. (Biol. Pharm. Bull (2010), 33(2), 330) disclose an extract of Forsythia viridissima containing a mixture of oleanolic acid, ursolic acid, betulinic acid and other components.

Oleanolic acid (O or OA), ursolic acid (U or UA) and betulinic acid (B or BA) are the three major triterpene components found in PBI-05204 (PBI-23; a supercritical fluid extract of Nerium oleander) and PBI-04711 (a triterpene-containing fraction of PBI-05204). We (two of the instant inventors) previously reported (Van Kanegan et al., in Nature Scientific Reports (May 2016), 6:25626. doi: 10.1038/srep25626) on the contribution of the triterpenes toward efficacy by comparing their neuroprotective activity in a brain slice oxygen glucose deprivation (OGD) model assay at similar concentrations. We found that PBI-05204 (PBI) and PBI-04711 (Fraction 0-4) provide neuroprotective activity.

Extracts of Nerium species are known to contain many different classes of compounds: cardiac glycosides, glycones, steroids, triterpenes, polysaccharides and others. Specific compounds include oleandrin; neritaloside; odoroside; oleanolic acid; ursolic acid; betulinic acid; oleandrigenin; oleaside A; betulin (urs-12-ene-3β,28-diol); 28-norurs-12-en-3β-ol; urs-12-en-3β-ol; 3β,3β-hydroxy-12-oleanen-28-oic acid; 3β,20α-dihydroxyurs-21-en-38-oic acid; 30,27-dihydroxy-12-ursen-38-oic acid; 3β,13β-dihydroxyurs-11-en-28-oic acid; 3β,12α-dihydroxyoleanan-28,13β-olide; 3β,27-dihydroxy-12-oleanan-28-oic acid; and other components.

Multhoff et al. (“Radiation, inflammation, and immune responses in Cancer” in Front. Onc. (June 2012), 2(58), 1-18; doi: 10.3389/fonc.2012.00058) suggest the use of oleandrin, ursolic acid or betulinic acid, among other compounds, as radiosensitizers for use in combination with radiotherapy.

Failure of standard of chemo/radiotherapy regimens has been attributed to multiple factors such as microenvironment protection, de novo and/or acquired tumor resistance, limitations in drug delivery, increased angiogenesis and/or vasculogenic mimicry (VM), and the facile emergence of glioma stem cells (GSCs) (Mooney et al., “Current Approaches and Challenges in the Molecular Therapeutic Targeting of Glioblastoma” in World neurosurgery, (2019), 129, 90-100).

A need remains for improved therapies to treat GBM, in particular recurrent or resistant GM.

### SUMMARY OF THE INVENTION

The invention provides a pharmaceutical composition and method for treating glioma (GM). As used herein, glioma can be selected from the group consisting of astrocytoma (which include low-grade astrocytomas (Grade I pilocytic astrocytoma, and Grade II difuse astrocytoma), anaplastic astrocytoma (Grade III), glioblastoma (Grade IV, GBM, also known as glioblastoma multiforme)), ependymoma, and oligodendroglioma. As used herein and unless otherwise specified, GM is intended to encompass all known grades of GM, and in particular GBM, meaning the GM can be any of Grade I, Grade II, Grade III or Grade IV.

As used herein and unless otherwise specified, GBM encompasses initial GBM (iGBM, which is GBM that is diagnosed (occurs) for the first time in a subject; this is also referred to as newly diagnosed GBM or nGBM) and/or recurrent (recurring) GBM (rGBM, which is GBM that is re-diagnosed (reccurs) in a subject having already had iGBM). The invention thus also provides a pharmaceutical composition and method for treating GM, in particular GBM (iGBM, rGBM). The GM (or GBM) can also be “treatment resistant GM” (trGM), “treatment resistant GBM” (trGBM), “recurrent GM” (rGM), or “recurrent GBM” (rGBM). Unless otherwise specified, GM should be construed to encompass all of these types.

The invention also provides a combination protocol for treating GM, in particular GBM.

The composition(s) and method(s) herein may provide increased overall survival (OS) and/or increased progression-free survival (PFS) and/or increased glioma-free survival (GFS) for subjects having GM or GBM.

The present invention provides a method of treating GM by administration of a pharmaceutical composition, which may comprise oleandrin, an extract comprising oleandrin, an extract of Nerium species, one or more components of an extract of Nerium species, or a mixture of at least three triterpenes (oleanolic acid (OA), ursolic acid (UA), betulinic acid (BA)) present at a combination of molar ratios (OA:UA:BA) as described herein. The composition optionally further comprises one or more other active ingredients. The one or more other active ingredients can be one or more active ingredients included in an extract containing oleandrin and/or one or more active ingredients known or found to be efficacious against GM.

In some embodiments, the extract, such as present in oleandrin-containing composition (OCC), may comprise one or more cardiac glycosides and one or more cardiac glycoside precursors (such as cardenolides, cardadienolides and cardatrienolides, all of which are the aglycone constituents of cardiac glycosides, for example, digitoxin, acetyl digitoxins, digitoxigenin, digoxin, acetyl digoxins, digoxigenin, medigoxin, strophanthins, cymarine, ouabain, or strophanthidin). The extract may further comprise one or more glycone constituents of cardiac glycosides (such as glucoside, fructoside, and/or glucuronide) as cardiac glycoside presursors. Accordingly, the composition may comprise one or more cardiac glycosides and two more cardiac glycoside precursors selected from the group consisting of one or more aglycone constituents, and one or more glycone constituents.

In some embodiments, the composition comprises one or more cardiac glycosides and one or more cardiac glycoside precursors (such as cardenolides, cardadienolides and cardatrienolides, all of which are the aglycone constituents of cardiac glycosides, for example, digitoxin, acetyl digitoxins, digitoxigenin, digoxin, acetyl digoxins, digoxigenin, medigoxin, strophanthins, cymarine, ouabain, or strophanthidin). The composition may further comprise one or more glycone constituents of cardiac glycosides (such as glucoside, fructoside, and/or glucuronide) as cardiac glycoside presursors. Accordingly, the composition may comprise one or more cardiac glycosides and two more cardiac glycoside precursors selected from the group consisting of one or more aglycone constituents, and one or more glycone constituents.

In some embodiments, the composition excludes oleandrin. Such a composition would comprise a mixture of triterpenes (MT) OA+UA+BA. Various improved triterpene mixtures, as well as their use, disclosed herein are considered within the scope of the invention.

In some embodiments, the composition comprises one or more components extractable from Nerium species plant material. In some embodiments, the composition comprises one or more components selected from the group consisting of cardiac glycoside, glycone, aglycone, steroid, triterpene, polysaccharide, saccharide, alkaloid, fat, protein, neritaloside, odoroside, oleanolic acid, ursolic acid, betulinic acid, oleandrigenin, oleaside A, betulin (urs-12-ene-3β,28-diol), 28-norurs-12-en-3β-ol, urs-12-en-3β-ol, 3β,3β-hydroxy-12-oleanen-28-oic acid, 3β,20α-dihydroxyurs-21-en-28-oic acid, 3β,27-dihydroxy-12-ursen-28-oic acid, 3β,13β-dihydroxyurs-11-en-28-oic acid, 3β,12α-dihydroxyoleanan-28,13β-olide, 3β,27-dihydroxy-12-oleanan-28-oic acid, homopolygalacturonan, arabinogalaturonan, chlorogenic acid, caffeic acid, L-quinic acid, 4-coumaroyl-CoA, 3-O-caffeoylquinic acid, 5-O-caffeoylquinic acid, cardenolide B-1, cardenolide B-2, oleagenin, neridiginoside, nerizoside, odoroside-H, 3-beta-O-(D-diginosyl)-5-beta, 14 beta-dihydroxy-card-20(22)-enolide pectic polysaccharide composed of galacturonic acid, rhamnose, arabinose, xylose, and galactose, polysaccharide with MW in the range of 17000-120000 D, or MW about 35000 D, about 3000 D, about 5500 D, or about 12000 D, cardenolide monoglycoside, cardenolide N-1, cardenolide N-2, cardenolide N-3, cardenolide N-4, pregnane, 4,6-diene-3,12,20-trione, 20R-hydroxypregna-4,6-diene-3,12-dione, 16beta,17beta-epoxy-12beta-hydroxypregna-4,6-diene-3,20-dione, 12beta-hydroxypregna-4,6,16-triene-3,20-dione (neridienone A), 20S,21-dihydroxypregna-4,6-diene-3,12-dione (neridienone B), neriucoumaric acid, isoneriucoumaric acid, oleanderoic acid, oleanderen, 8alpha-methoxylabdan-18-oic acid, 12-ursene, kaneroside, neriumoside, 3β-O-(D-diginosyl)-2α-hydroxy-8,14β-epoxy-5β-carda-16:17,20:22-dienolide, 3β-O-(D-diginosyl)-2α,14β-dihydroxy-5β-carda-16:17,20:22-dienolide, 3β,27-dihydroxy-urs-18-en-13,28-olide, 3β,22α,28-trihydroxy-25-nor-lup-1(10),20(29)-dien-2-one, cis-karenin (3β-hydroxy-28-Z-p-coumaroyloxy-urs-12-en-27-oic acid), trans-karenin (3-β-hydroxy-28-E-p-coumaroyloxy-urs-12-en-27-oic acid), 3beta-hydroxy-5alpha-carda-14(15),20(22)-dienolide (beta-anhydroepidigitoxigenin), 3 beta-O-(D-digitalosyl)-21-hydroxy-5beta-carda-8,14,16,20(22)-tetraenolide (neriumogenin-A-3beta-D-digitaloside), proceragenin, neridienone A, 3beta,27-dihydroxy-12-ursen-28-oic acid, 3beta,13beta-dihydroxyurs-11-en-28-oic acid, 3beta-hydroxyurs-12-en-28-aldehyde, 28-orurs-12-en-3beta-ol, urs-12-en-3beta-ol, urs-12-ene-3beta,28-diol, 3beta,27-dihydroxy-12-oleanen-28-oic acid, (20S,24R)-epoxydammarane-3beta,25-diol, 20beta,28-epoxy-28alpha-methoxytaraxasteran-3beta-ol, 20beta,28-epoxytaraxaster-21-en-3beta-ol, 28-nor-urs-12-ene-3beta,17 beta-diol, 3beta-hydroxyurs-12-en-28-aldehyde, alpha-neriursate, beta-neriursate, 3alpha-acetophenoxy-urs-12-en-28-oic acid, 3beta-acetophenoxy-urs-12-en-28-oic acid, oleanderolic acid, kanerodione, 3β-p-hydroxyphenoxy-11α-methoxy-12α-hydroxy-20-ursen-28-oic acid, 28-hydroxy-20(29)-lupen-3,7-dione, kanerocin, 3alpha-hydroxy-urs-18,20-dien-28-oic acid, D-sarmentose, D-diginose, neridiginoside, nerizoside, isoricinoleic acid, gentiobiosylnerigoside, gentiobiosylbeaumontoside, gentiobiosyloleandrin, folinerin, 12β-hydroxy-5β-carda-8,14,16,20(22)-tetraenolide, 8β-hydroxy-digitoxigenin, Δ16-8β-hydroxy-digitoxigenin, Δ16-neriagenin, uvaol, ursolic aldehyde, 27(p-coumaroyloxy)ursolic acid, oleanderol, 16-anhydro-deacteyl-nerigoside, 9-D-hydroxy-cis-12-octadecanoic acid, adigoside, adynerin, alpha-amyrin, beta-sitosterol, campestrol, caoutchouc, capric acid, caprylic acid, choline, cornerin, cortenerin, deacetyloleandrin, diacetyl-nerigoside, foliandrin, pseudocuramine, quercetin, quercetin-3-rhamnoglucoside, quercitrin, rosaginin, rutin, stearic acid, stigmasterol, strospeside, urehitoxin, and uzarigenin. Additional components that may be present in the extract are disclosed by Gupta et al. (IJPSR (2010), 1(3), 21-27, the entire disclosure of which is hereby incorporated by reference).

An extract, such as present in OCC, may comprise oleandrin and one or more components selected from the group consisting of glycoside, glycone, aglycone, steroid, triterpene, polysaccharide, saccharide, alkaloid, fat, protein, neritaloside, odoroside, oleanolic acid, ursolic acid, betulinic acid, oleandrigenin, oleaside A, betulin (urs-12-ene-3β,28-diol), 28-norurs-12-en-3β-ol, urs-12-en-3β-ol, 3β,3β-hydroxy-12-oleanen-28-oic acid, 3β,20α-dihydroxyurs-21-en-28-oic acid, 3β,27-dihydroxy-12-ursen-28-oic acid, 3β,13β-dihydroxyurs-11-en-28-oic acid, 3β,12α-dihydroxyoleanan-28,13β-olide, 3β,27-dihydroxy-12-oleanan-28-oic acid, homopolygalacturonan, arabinogalaturonan, chlorogenic acid, caffeic acid, L-quinic acid, 4-coumaroyl-CoA, 3-O-caffeoylquinic acid, 5-O-caffeoylquinic acid, cardenolide B-1, cardenolide B-2, oleagenin, neridiginoside, nerizoside, odoroside-H, 3-beta-O-(D-diginosyl)-5-beta, 14 beta-dihydroxy-card-20(22)-enolide pectic polysaccharide composed of galacturonic acid, rhamnose, arabinose, xylose, and galactose, polysaccharide with MW in the range of 17000-120000 D, or MW about 35000 D, about 3000 D, about 5500 D, or about 12000 D, cardenolide monoglycoside, cardenolide N-1, cardenolide N-2, cardenolide N-3, cardenolide N-4, pregnane, 4,6-diene-3,12,20-trione, 20R-hydroxypregna-4,6-diene-3,12-dione, 16beta,17beta-epoxy-12beta-hydroxypregna-4,6-diene-3,20-dione, 12beta-hydroxypregna-4,6,16-triene-3,20-dione (neridienone A), 20S,21-dihydroxypregna-4,6-diene-3,12-dione (neridienone B), neriucoumaric acid, isoneriucoumaric acid, oleanderoic acid, oleanderen, 8alpha-methoxylabdan-18-oic acid, 12-ursene, kaneroside, neriumoside, 3β-O-(D-diginosyl)-2α-hydroxy-8,14β-epoxy-5β-carda-16:17,20:22-dienolide, 3β-O-(D-diginosyl)-2α,14β-dihydroxy-5β-carda-16:17,20:22-dienolide, 3β,27-dihydroxy-urs-18-en-13,28-olide, 3β,22α,28-trihydroxy-25-nor-lup-1(10),20(29)-dien-2-one, cis-karenin (3β-hydroxy-28-Z-p-coumaroyloxy-urs-12-en-27-oic acid), trans-karenin (3-β-hydroxy-28-E-p-coumaroyloxy-urs-12-en-27-oic acid), 3beta-hydroxy-5alpha-carda-14(15),20(22)-dienolide (beta-anhydroepidigitoxigenin), 3 beta-O-(D-digitalosyl)-21-hydroxy-5beta-carda-8,14,16,20(22)-tetraenolide (neriumogenin-A-3beta-D-digitaloside), proceragenin, neridienone A, 3beta,27-dihydroxy-12-ursen-28-oic acid, 3beta,13beta-dihydroxyurs-11-en-28-oic acid, 3beta-hydroxyurs-12-en-28-aldehyde, 28-orurs-12-en-3beta-ol, urs-12-en-3beta-ol, urs-12-ene-3beta,28-diol, 3beta,27-dihydroxy-12-oleanen-28-oic acid, (20S,24R)-epoxydammarane-3beta,25-diol, 20beta,28-epoxy-28alpha-methoxytaraxasteran-3beta-ol, 20beta,28-epoxytaraxaster-21-en-3beta-ol, 28-nor-urs-12-ene-3beta,17 beta-diol, 3beta-hydroxyurs-12-en-28-aldehyde, alpha-neriursate, beta-neriursate, 3alpha-acetophenoxy-urs-12-en-28-oic acid, 3beta-acetophenoxy-urs-12-en-28-oic acid, oleanderolic acid, kanerodione, 3β-p-hydroxyphenoxy-11α-methoxy-12α-hydroxy-20-ursen-28-oic acid, 28-hydroxy-20(29)-lupen-3,7-dione, kanerocin, 3alpha-hydroxy-urs-18,20-dien-28-oic acid, D-sarmentose, D-diginose, neridiginoside, nerizoside, isoricinoleic acid, gentiobiosylnerigoside, gentiobiosylbeaumontoside, gentiobiosyloleandrin, folinerin, 12β-hydroxy-5β-carda-8,14,16,20(22)-tetraenolide, 8β-hydroxy-digitoxigenin, Δ16-8β-hydroxy-digitoxigenin, Δ16-neriagenin, uvaol, ursolic aldehyde, 27(p-coumaroyloxy)ursolic acid, oleanderol, 16-anhydro-deacteyl-nerigoside, 9-D-hydroxy-cis-12-octadecanoic acid, adigoside, adynerin, alpha-amyrin, beta-sitosterol, campestrol, caoutchouc, capric acid, caprylic acid, choline, cornerin, cortenerin, deacetyloleandrin, diacetyl-nerigoside, foliandrin, pseudocuramine, quercetin, quercetin-3-rhamnoglucoside, quercitrin, rosaginin, rutin, stearic acid, stigmasterol, strospeside, urehitoxin, and uzarigenin. Additional components that may be present in the extract are disclosed by Gupta et al. (IJPSR (2010), 1(3), 21-27, the entire disclosure of which is hereby incorporated by reference).

Some embodiments concern treatment of GM using one or more combination protocols comprising radiotherapy, chemotherapy, and pharmacotherapy (administration of pharmaceutical composition(s) as described herein). Any combination protocol optionally further comprises resection of the GM tumor.

The invention provides one or more combination protocols for the treatment of GM, in particular GBM. A combination protocol for treating a subject with GM comprises at least the following steps, which may be executed in any order during a treatment period:
treating said subject with radiotherapy;
treating said subject with chemotherapy;
treating said subject with pharmacotherapy (administration of composition(s), pharmaceutical composition(s) or oleandring-containing composition(s) as described herein); and
optionally resecting the GM.

A combination protocol for treating a subject with GM comprises at least the following steps, which may be executed in any order during a treatment period:
treating said subject with radiotherapy;
treating said subject with pharmacotherapy (administration of composition(s) or pharmaceutical composition(s) as described herein); and
optionally resecting the GM.

A combination protocol for treating a subject with GM comprises at least the following steps, which may be executed in any order during a treatment period:
treating said subject with chemotherapy;
treating said subject with pharmacotherapy (administration of composition(s) or pharmaceutical composition(s) as described herein); and
optionally resecting the GM.

A combination protocol for the treatment of GM, in particular GBM, in a subject, said protocol comprising
resecting said GM, or GBM, from said subject, thereby leaving a resection site in said subject; and
conducting the following steps in an overlapping manner during a treatment period:
   administering TMZ to said subject according to any one or more of the dosing protocols described herein;
   irradiating tissue defining and surrounding said resection site with X-ray radiation according to any one or more of the dosing protocols described herein; and
   administering a composition as described herein to said subject according to any one or more of the dosing protocols described herein.

Some embodiments of the invention include those wherein a) radiotherapy is conducted repeatedly during a treatment period; b) chemotherapy is conducted repeatedly during a treatment period; c) pharmacotherapy is conducted repeatedly during a treatment period; d) radiotherapy and chemotherapy are conducted in an overlapping manner during a treatment period; e) radiotherapy and pharmacotherapy are conducted in an overlapping manner during a treatment period; f) chemotherapy and pharmacotherapy are conducted in an overlapping manner during a treatment period; g) radiotherapy, chemotherapy and pharmacotherapy are conducted in an overlapping manner during a treatment period; h) radiotherapy and pharmacotherapy are conducted in a sequential manner during a treatment period; i) chemotherapy and pharmacotherapy are conducted in a sequential manner during a treatment period; j) radiotherapy, chemotherapy and pharmacotherapy are conducted in a sequential manner during a treatment period; k) resection of the tumor is conducted before any one of radiotherapy, chemotherapy and pharmacotherapy; l) resection of the tumor is conducted after any one of radiotherapy, chemotherapy and pharmacotherapy; m) resection of the tumor is conducted between radiotherapy and chemotherapy; n) resection of the tumor is conducted between radiotherapy and pharmacotherapy; o) resection of the tumor is conducted between chemotherapy and pharmacotherapy, or p) any combination of the above.

In some embodiments, the invention provides a combination protocol for treating a subject with GM, said protocol comprising resecting the GM and then conducting at least the following steps in any order during a treatment period:
treating said subject with radiotherapy;
treating said subject with chemotherapy; and
treating said subject with pharmacotherapy (administration of composition(s), pharmaceutical composition(s), or oleandrin-containing composition(s) as described herein).

In some embodiments, the invention provides a combination protocol for treating a subject with GM, said protocol comprising resecting the GM and then conducting at least the following steps in any order during a treatment period:
treating said subject with X-ray radiotherapy;
treating said subject with TMZ chemotherapy; and
treating said subject with pharmacotherapy by administering composition(s), pharmaceutical composition(s), or oleandrin-containing pharmaceutical composition(s).

In some embodiments, the invention provides a combination protocol for treating a subject with GM, said protocol comprising resecting the GM and then conducting at least the following steps in any order during a treatment period:
treating said subject with X-ray radiotherapy;
treating said subject with TMZ chemotherapy; and
treating said subject with pharmacotherapy by administering: a) a pharmaceutical composition comprising oleandrin; b) a pharmaceutical composition comprising extract of plant material, said extract comprising oleandrin; c) a pharmaceutical composition comprising extract of plant material, said extract comprising oleandrin and one or more other active ingredients extractable from said plant material; d) a pharmaceutical composition comprising one or more components extractable from Nerium species; or e) any combination thereof.

The extract can be obtained by supercritical fluid (SCF) extraction, water extraction (hot or cold water extraction), or organic solvent extraction of plant material. The extract can be the result of extraction using a combination of any two of SCF extraction, water extraction, or organic solvent extraction of plant material. In some embodiments, the plant material is obtained from Nerium sp.

In some embodiments, the invention provides a combination protocol for treating a subject with GM, said protocol comprising resecting the GM and then conducting at least the following steps in any order during a treatment period:
irradiating said subject with X-ray according to any one or more of the dosing protocols described herein;
administering TMZ to said subject according any one or more of the dosing protocols described herein; and
administering oleandrin to said subject, said oleandrin being present in: a) a pharmaceutical composition comprising oleandrin; b) a pharmaceutical composition comprising extract of plant material, said extract comprising oleandrin; c) a pharmaceutical composition comprising extract of plant material, said extract comprising oleandrin and one or more other active ingredients extractable from said plant material; or d) any combination thereof.

In all embodiments of the invention described herein, the glioma can be GM or GBM that is newly diagnosed, recurrent, or treatment resistant.

The invention also provides a method of treating recurrent or treatment resistant GM (or GBM) comprising administering to a subject in need thereof a pharmaceutical composition comprising a) oleandrin; b) an extract comprising oleandrin; c) an extract of Nerium species; d) one or more components of an extract of Nerium species; or e) a mixture of at least three triterpenes (oleanolic acid (OA), ursolic acid (UA), betulinic acid (BA)) present at a combination of molar ratios (OA:UA:BA), as described herein. The composition optionally further comprises one or more other active ingredients. The one or more other active ingredients can be one or more active ingredients included in an extract containing oleandrin and/or one or more active ingredients known or found to be efficacious against GM.

The invention also provides a combination protocol for treating a subject having recurrent or treatment resistant GM (or GBM), the method comprising at least the following steps, which may be executed in any order during a treatment period:
treating said subject with radiotherapy;
treating said subject with chemotherapy;
treating said subject with pharmacotherapy (administration of composition(s), pharmaceutical composition(s) or oleandring-containing composition(s) as described herein); and
optionally resecting the GM.

The invention also provides A combination protocol for the treatment of recurrent or treatment resistant GM, in particular GBM, in a subject, said protocol comprising
resecting said GM, or GBM, from said subject, thereby leaving a resection site in said subject; and
conducting the following steps in an overlapping manner during a treatment period:
   administering TMZ to said subject according to any one or more of the dosing protocols described herein;
   irradiating tissue defining and surrounding said resection site with X-ray radiation according to any one or more of the dosing protocols described herein; and
   administering a composition as described herein to said subject according to any one or more of the dosing protocols described herein.

During a method of treatment of the invention, administration of the composition as described herein will result in a reduction of the number and/or size of spheroids of glioma stem cells (in particular, GBM stem cells) in the subject, in particular if the GM or GBM is treatment resistant.

In some embodiments, the OCC (or composition or pharmaceutical composition) is administered chronically in daily doses over a period of days. The OCC (or composition or pharmaceutical composition) can be administered chronically daily over a period of at least one week, at least two weeks, at least three weeks, at least four weeks, at least five weeks, at least six weeks or more. A subject can also be administered maintenance doses of OCC (or composition or pharmaceutical composition) after completion of radiotherapy and chemotherapy. In some embodiments, a subject is administered daily dose of OCC (or composition or pharmaceutical composition) for a period of at least four weeks or at least one month. In some embodiments, a subject is administered daily doses of OCC (or composition or pharmaceutical composition) for at least a first period of days, weeks, or months. In some embodiments, a subject is not administered daily doses of OCC (or composition or pharmaceutical composition) for at least one day or for at least a period of 1-7, 1-6, 1-5, 1-4, 1-3, or 1-2 days. Combinations of all OCC (or composition or pharmaceutical composition) dosing regimens disclosed in this application or found to be safe and effective are contemplated within the scope of the invention.

In some embodiments, radiotherapy includes exposing the subject to irradiation with X-rays, such as by external beam radiation. Some embodiments of the invention include those wherein a) the subject receives a dose of X-ray radiation daily for at least one day or for a first period of 1-7, 1-6, 1-5, 1-4, 1-3, or 1-2 days; b) the subject is not exposed to X-ray radiation for at least one day or for a second period of 1-2, 1-3, 1-4, 1-5, 1-6, or 1-7 days; and optionally c) steps (items) a) and b) are repeated at least once, at least twice, at least three time, as least four times, at least five times, at least six times. Items a) (first period) and b) (second period) can be repeated as many times as needed to provide the target clinical benefit. In some embodiments, radiotherapy is dose fractionated, whereby a total dose radiation is divided and administered over a predetermined period of days. For example, a total dose of about 60 Gy is administered in about 30 daily fractions, or a total dose of about 60 Gy is administered in about 2 Gy fractions over a period of about 30 days, over a period of about 4 to about 7 weeks, over a period of about 4 to about 6 weeks, over a period of about 5 to about 7 weeks, or over a period of about 6 weeks.

Alternatively, a RT (otherwise referred to as XRT) protocol can be as follows: a 2 cm CTV (computed tomographic venography) margin and 3-5 mm PTV (planning target volume) margin followed by a “conedown” (also called a “boost”) phase with a more limited volume, defined by the contrast-enhanced T1 abnormality on postoperative MRI with a 2-cm CTV margin and a 3- to 5-mm PTV margin. In some embodiments, the initial volume receives about 46 Gy in about 2 Gy daily fractions, and the boost volume receives about 14 Gy in about 2 Gy daily fractions. Alternatively, high-grade glioma can be treated with fractionated stereotactic RT (median dose, about 35 Gy in about 10 fractions). Also, for previously irradiated patients with recurrent GBM can be administered a median total dose of about 30 Gy (median about 5 Gy/fraction). Elderly patients with GBM can be treated with hypofractionated RT given as about 40 Gy in about 15 fractions over 3 weeks with concomitant temozolamide and/or adjuvant temozolamide at doses as described herein. For elderly patients over the age of 70 with GBM or anaplastic astrocytoma (AA) with a KPS of 70 or more, they may be treated with focal RT by administering about 50.4 Gy given in daily fractions of about 1.8 Gy. An alternative RT dosing protocol can include a median dose of about 36 Gy in about 2-Gy fractions. Alternatively, a suitable RT dosing schedule can include fractionated stereotactic RT by administering a median dose of about 36 Gy in about 2-Gy fractions.

In some embodiments, TMZ is administered at about 75 mg/m² daily for about 42 days concomitant with focal radiotherapy (about 60 Gy administered in about 30 fractions) followed by maintenance dose of TMZ for about 6 cycles. Focal RT includes the tumor bed or resection site with an about 2 to about 3 cm margin. No dose reductions are recommended during the concomitant phase; however, dose interruptions or discontinuation may occur based on toxicity. The TMZ dose should be continued throughout the about 42-day concomitant period up to about 49 days. Four weeks after completing the TMZ+RT phase, TMZ is administered for an additional 6 cycles of maintenance treatment. Cycles 1-6: a) Cycle 1—Dosage in Cycle 1 (maintenance) is about 150 mg/m² once daily for 5 days followed by about 23 days without treatment; b) at the start of Cycle 2, the dose can be escalated to about 200 mg/m², if the CTC nonhematologic toxicity for Cycle 1 is Grade less than or equal to 2 (except for alopecia, nausea, and vomiting), absolute neutrophil count (ANC) is greater than or equal to about 1.5×10⁹/L, and the platelet count is greater than or equal to about 100×10⁹/L. The dose remains at about 200 mg/m² per day for the first about 5 days of each subsequent cycle except if toxicity occurs. If the dose was not escalated at Cycle 2, escalation should not be done in subsequent cycles. Dose reductions during the maintenance phase should be applied according to the following table.

TABLE 1
Temozolomide Dose Levels for Maintenance Treatment:

| Dose Level | Dose (mg/m²/day) | Remarks |
|---|---|---|
| −1 | 100 | Reduction for prior toxicity |
| 0 | 150 | Dose during Cycle 1 |
| 1 | 200 | Dose during Cycles 2-6 in |
| | | absence of toxicity |

TABLE 2
Temozolomide Dose Reduction or Discontinuation During
Maintenance Treatment

| | Reduce TMZ by 1 | Discontinue |
|---|---|---|
| Toxicity | Dose Level* | TMZ |
| Absolute Neutrophil Count | less than 1.0 × 109/L | See footnote† |
| Platelet Count | less than 50 × 109/L | See footnote† |
| CTC Nonhematological Toxicity | CTC Grade 3 | CTC Grade 4† |
| (except for alopecia, nausea, | | |
| vomiting) | | |

*TMZ dose levels are listed in Table 1.
†TMZ is to be discontinued if dose reduction to less than 100 mg/m² is required or if the same Grade 3 nonhematological toxicity (except for alopecia, nausea, vomiting) recurs after dose reduction.
TMZ = temozolomide;
CTC = Common Toxicity Criteria.

For patients with refractory astrocytoma, a suitable TMZ treatment schedule can be as follows. For adults the initial dose can be about 150 mg/m² once daily for about 5 consecutive days per 28-day treatment cycle. For adult patients, if both the nadir and day of dosing (Day 29, Day 1 of next cycle) ANC are greater than or equal to about 1.5×10⁹/L (1500/μL) and both the nadir and Day 29, Day 1 of next cycle platelet counts are greater than or equal to about 100×10⁹/L (100,000/μL), the TMZ dose may be increased to about 200 mg/m²/day for about 5 consecutive days per 28-day treatment cycle.

The daily dose calculations for TMZ administration according to body surface area (BSA) can be as follows.

| Total BSA | 75 mg/m² | 150 mg/m² | 200 mg/m² |
|---|---|---|---|
| (m²) | (mg daily) | (mg daily) | (mg daily) |
| 1.0 | 75 | 150 | 200 |
| 1.1 | 82.5 | 165 | 220 |
| 1.2 | 90 | 180 | 240 |
| 1.3 | 97.5 | 195 | 260 |
| 1.4 | 105 | 210 | 280 |
| 1.5 | 112.5 | 225 | 300 |
| 1.6 | 120 | 240 | 320 |
| 1.7 | 127.5 | 255 | 340 |
| 1.8 | 135 | 270 | 360 |
| 1.9 | 142.5 | 285 | 380 |
| 2.0 | 150 | 300 | 400 |
| 2.1 | 157.5 | 315 | 420 |
| 2.2 | 165 | 330 | 440 |
| 2.3 | 172.5 | 345 | 460 |
| 2.4 | 180 | 360 | 480 |
| 2.5 | 187.5 | 375 | 500 |

TMZ-containing capsules in various dosage strengths (5 mg, 20 mg, 100 mg, 140 mg, 180 mg and 250 mg under the tradename TEMODAR® (Merck & Co., Inc., Whitehouse Station, N.J. 08889, USA; NDA 021029, the entire disclosure of which is hereby incorporated by reference) are preferably administered on an empty stomach. Bedtime administration may be advised. TMZ can also be administered by injection in an aqueous vehicle comprising about 2.5 mg of TMZ/mL. The drug-containing solution should be administered by intravenous infusion. Additional prescribing information for TMZ is available under NDA 021029, the entire disclosure of which is incorporated herein by reference.

In some embodiments, the invention provides use of an anticancer composition for the treatment of GM, said anticancer composition comprising (consisting essentially of): a) one or more cardiac glycoside(s); b) one or more triterpenes; c) one or more chemotherapeutic agent(s); and/or d) any combination of the listed items.

Chemotherapy is intended to include at least administering to a subject one or more chemotherapeutic agents known or found to be therapeutically effective against GM, esp. GBM. In some embodiments, the one or more other chemotherapeutic agents are selected from the group consisting of nitrosoureas, DNA alkylating agent(s), temozolomide, carmustine (BCNU), lomustine (CCNU), nimustine (ACNU), fotemusine, cediranib, erlotinib, galunisertib, irinotecan, procarbazine, vincristine, bevacizumab, hydroxyurea, and cytarabine.

Pharmacotherapy is intended to include at least administering to a subject a composition comprising: a) oleandrin; b) oleandrin-containing extract; c) oleandrin and one or more other active ingredients extractable from oleander plant; d) a triterpene composition comprising OA, UA, and BA; or e) any combination thereof. Pharmacotherapy can further comprise a) administering one or more drugs for treating the symptoms associated with GM (or GBM); b) administering one or more drugs for treating adverse event(s) associated radiotherapy; c) administering one or more drugs for treating adverse event(s) associated with chemotherapy; or d) any combination thereof.

Exemplary one or more active ingredients extractable from Nerium species, e.g. oleander plant can be selected from the group consisting of glycoside, glycone, aglycone, steroid, triterpene, polysaccharide, saccharide, alkaloid, fat, protein, neritaloside, odoroside, oleanolic acid, ursolic acid, betulinic acid, oleandrigenin, oleaside A, betulin (urs-12-ene-3β,28-diol), 28-norurs-12-en-3β-ol, urs-12-en-3β-ol, 3β,3β-hydroxy-12-oleanen-28-oic acid, 3β,20α-dihydroxyurs-21-en-28-oic acid, 3β,27-dihydroxy-12-ursen-28-oic acid, 3β,13β-dihydroxyurs-11-en-28-oic acid, 3β,12α-dihydroxyoleanan-28,13β-olide, 3β,27-dihydroxy-12-oleanan-28-oic acid, homopolygalacturonan, arabinogalaturonan, chlorogenic acid, caffeic acid, L-quinic acid, 4-coumaroyl-CoA, 3-O-caffeoylquinic acid, 5-O-caffeoylquinic acid, cardenolide B-1, cardenolide B-2, oleagenin, neridiginoside, nerizoside, odoroside-H, 3-beta-O-(D-diginosyl)-5-beta, 14 beta-dihydroxy-card-20(22)-enolide pectic polysaccharide composed of galacturonic acid, rhamnose, arabinose, xylose, and galactose, polysaccharide with MW in the range of 17000-120000 D, or MW about 35000 D, about 3000 D, about 5500 D, or about 12000 D, cardenolide monoglycoside, cardenolide N-1, cardenolide N-2, cardenolide N-3, cardenolide N-4, pregnane, 4,6-diene-3,12,20-trione, 20R-hydroxypregna-4,6-diene-3,12-dione, 16beta,17beta-epoxy-12beta-hydroxypregna-4,6-diene-3,20-dione, 12beta-hydroxypregna-4,6,16-triene-3,20-dione (neridienone A), 20S,21-dihydroxypregna-4,6-diene-3,12-dione (neridienone B), neriucoumaric acid, isoneriucoumaric acid, oleanderoic acid, oleanderen, 8alpha-methoxylabdan-18-oic acid, 12-ursene, kaneroside, neriumoside, 3β-O-(D-diginosyl)-2α-hydroxy-8,14β-epoxy-5β-carda-16:17,20:22-dienolide, 3β-O-(D-diginosyl)-2α,14β-dihydroxy-5β-carda-16:17,20:22-dienolide, 3β,27-dihydroxy-urs-18-en-13,28-olide, 3β,22α,28-trihydroxy-25-nor-lup-1(10),20(29)-dien-2-one, cis-karenin (3β-hydroxy-28-Z-p-coumaroyloxy-urs-12-en-27-oic acid), trans-karenin (3-β-hydroxy-28-E-p-coumaroyloxy-urs-12-en-27-oic acid), 3beta-hydroxy-5alpha-carda-14(15),20(22)-dienolide (beta-anhydroepidigitoxigenin), 3 beta-O-(D-digitalosyl)-21-hydroxy-5beta-carda-8,14,16,20(22)-tetraenolide (neriumogenin-A-3beta-D-digitaloside), proceragenin, neridienone A, 3beta,27-dihydroxy-12-ursen-28-oic acid, 3beta,13beta-dihydroxyurs-11-en-28-oic acid, 3beta-hydroxyurs-12-en-28-aldehyde, 28-orurs-12-en-3beta-ol, urs-12-en-3beta-ol, urs-12-ene-3beta,28-diol, 3beta,27-dihydroxy-12-oleanen-28-oic acid, (20S,24R)-epoxydammarane-3beta,25-diol, 20beta,28-epoxy-28alpha-methoxytaraxasteran-3beta-ol, 20beta,28-epoxytaraxaster-21-en-3beta-ol, 28-nor-urs-12-ene-3beta,17 beta-diol, 3beta-hydroxyurs-12-en-28-aldehyde, alpha-neriursate, beta-neriursate, 3alpha-acetophenoxy-urs-12-en-28-oic acid, 3beta-acetophenoxy-urs-12-en-28-oic acid, oleanderolic acid, kanerodione, 3β-p-hydroxyphenoxy-11α-methoxy-12α-hydroxy-20-ursen-28-oic acid, 28-hydroxy-20(29)-lupen-3,7-dione, kanerocin, 3alpha-hydroxy-urs-18,20-dien-28-oic acid, D-sarmentose, D-diginose, neridiginoside, nerizoside, isoricinoleic acid, gentiobiosylnerigoside, gentiobiosylbeaumontoside, gentiobiosyloleandrin, folinerin, 12β-hydroxy-5β-carda-8,14,16,20(22)-tetraenolide, 8β-hydroxy-digitoxigenin, Δ16-8β-hydroxy-digitoxigenin, Δ16-neriagenin, uvaol, ursolic aldehyde, 27(p-coumaroyloxy)ursolic acid, oleanderol, 16-anhydro-deacteyl-nerigoside, 9-D-hydroxy-cis-12-octadecanoic acid, adigoside, adynerin, alpha-amyrin, beta-sitosterol, campestrol, caoutchouc, capric acid, caprylic acid, choline, cornerin, cortenerin, deacetyloleandrin, diacetyl-nerigoside, foliandrin, pseudocuramine, quercetin, quercetin-3-rhamnoglucoside, quercitrin, rosaginin, rutin, stearic acid, stigmasterol, strospeside, urehitoxin, and uzarigenin. Additional components that may be present in the extract are disclosed by Gupta et al. (IJPSR (2010), 1(3), 21-27, the entire disclosure of which is hereby incorporated by reference).

Any treatment method of the invention can comprise dose escalation or dose de-escalation as needed. For example, the following:
administering an initial dose of a composition to the subject according to a prescribed initial dosing regimen for a period of time;
periodically determining the adequacy of subject's clinical response and/or therapeutic response to treatment with said composition; and
if the subject's clinical response and/or therapeutic response is adequate, then continuing treatment with said composition as needed until the desired clinical endpoint is achieved; or
if the subject's clinical response and/or therapeutic response are inadequate at the initial dose and initial dosing regimen, then escalating or deescalating the dose until the desired clinical response and/or therapeutic response in the subject is achieved.

Treatment of a subject with a composition is continued as needed. The dose or dosing regimen can be adjusted as needed until the patient reaches the desired clinical endpoint(s). Determination of the adequacy of clinical response and/or therapeutic response can be conducted by a clinician familiar with GM.

The individual steps of the methods of the invention can be conducted at separate facilities or within the same facility.

It is contemplated that any composition as described herein can be administered chronically, i.e. on a recurring basis, such as daily, every other day, every second day, every third day, every fourth day, every fifth day, every sixth day, weekly, every other week, every second week, every third week, monthly, bimonthly, semi-monthly, every other month every second month, quarterly, every other quarter, trimesterly, seasonally, semi-annually and/or annually. The composition can be administered daily for a first period of days (1-6, 1-5, 1-4, 1-3, 1-2, or 1 day(s)) and then not administered for a second period of days (1-6, 1-5, 1-4, 1-3, 1-2, or 1 day(s)).

In some embodiments, the subject is administered 140 microg to 315 microg of oleandrin per day. In some embodiments, a dose comprises 20 microg to 750 microg, 12 microg to 300 microg, or 12 microg to 120 microg of oleandrin. The daily dose of oleandrin can range from 20 microg to 750 microg, 0.01 microg to 100 mg, or 0.01 microg to 100 microg of oleandrin/day. The recommended daily dose of oleandrin, present in the SCF extract, is generally about 0.25 to about 50 microg twice daily or about 0.9 to 5 microg twice daily or about every 12 hours. The dose can be about 0.5 to about 100 microg/day, about 1 to about 80 microg/day, about 1.5 to about 60 microg/day, about 1.8 to about 60 microg/day, about 1.8 to about 40 microg/day. The maximum tolerated dose can be about 100 microg/day, about 80 microg/day, about 60 microg/day, about 40 microg/day, about 38.4 microg/day or about 30 microg/day of oleander extract containing oleandrin and the minimum effective dose can be about 0.5 microg/day, about 1 microg/day, about 1.5 microg/day, about 1.8 microg/day, about 2 microg/day, or about 5 microg/day. Suitable doses comprising oleandrin and triterpene can be about 0.05-0.5 mg/kg/day, about 0.05-0.35 mg/kg/day, about 0.05-0.22 mg/kg/day, about 0.05-0.4 mg/kg/day, about 0.05-0.3 mg/kg/day, about 0.05-0.5 microg/kg/day, about 0.05-0.35 microg/kg/day, about 0.05-0.22 microg/kg/day, about 0.05-0.4 microg/kg/day, or about 0.05-0.3 microg/kg/day.

Any composition described herein can be administered systemically. Modes of systemic administration include parenteral, buccal, enteral, intramuscular, subdermal, sublingual, peroral, or oral. The composition can also be administered via injection or intravenously.

The cardiac glycoside is preferably oleandrin. In some embodiments, the composition comprises or further comprises a) one or more triterpenes; b) one or more steroids; c) one or more triterpene derivatives; d) one or more steroid derivatives; or e) a combination thereof. In some embodiments, the composition comprises cardiac glycoside and: a) two or three triterpenes; b) two or three triterpene derivatives; c) two or three triterpene salts; or d) a combination thereof. In some embodiments, the triterpene is selected from the group consisting of oleanolic acid, ursolic acid, betulinic acid and salts, prodrugs, or derivatives thereof. As used herein, the generic terms triterpene and cardiac glycoside also encompass salts and derivatives thereof, unless otherwise specified.

Some embodiments of the invention include those wherein a pharmaceutical composition comprises at least one pharmaceutical excipient and oleadrin-containing composition. Some embodiments of the invention include those wherein a pharmaceutical composition comprises at least one pharmaceutical excipient and one or more components extractable from Nerium species.

The cardiac glycoside can be present in a pharmaceutical composition in pure form or as part of an extract comprising one or more cardiac glycosides. The triterpene(s) can be present in a pharmaceutical composition in pure form or as part of an extract comprising said triterpene(s). In some embodiments, the one or more components extractable from Nerium species are present in a pharmaceutical composition in pure form or as part of an extract comprising said component(s).

In some embodiments, the cardiac glycoside is present as the primary therapeutic component, meaning the component primarily responsible for anticancer activity, in the pharmaceutical composition. In some embodiments, the one or more components extractable from Nerium species are present as the primary therapeutic component. In some embodiments, the mixture of the triterpenes is present as the primary therapeutic component.

In some embodiments, an extract is obtained by extraction of plant material. The extract can comprise a hot-water extract, cold-water extract, supercritical fluid (SCF) extract, organic solvent extract, or combination thereof of the plant material. In some embodiments, the plant material is Nerium species plant mass. Particular species include Nerium oleander. In some embodiments, the extract comprises at least one pharmacologically active agent that contributes to the therapeutic efficacy of the composition when the extract is administered to a subject. In some embodiments, the composition further comprises one or more other non-cardiac glycoside therapeutically effective agents, i.e. one or more agents that are not cardiac glycosides.

In some embodiments, a composition comprising oleandrin (OL), oleanolic acid (OA), ursolic acid (UA) and betulinic acid (BA) is more efficacious than pure oleandrin, when equivalent doses based upon oleandrin content are compared.

In some embodiments, the molar ratio of total triterpene content (OA+UA+BA) to oleandrin ranges from about 15:1 to about 5:1, or about 12:1 to about 8:1, or about 100:1 to about 15:1, or about 100:1 to about 50:1, or about 100:1 to about 75:1, or about 100:1 to about 80:1, or about 100:1 to about 90:1, or about 10:1.

In some embodiments, the molar ratios of the individual triterpenes to oleandrin range as follows: about 2-8 (OA):about 2-8 (UA):about 0.1-1 (BA):about 0.5-1.5 (OL); or about 3-6 (OA):about 3-6 (UA):about 0.3-8 (BA):about 0.7-1.2 (OL); or about 4-5 (OA):about 4-5 (UA):about 0.4-0.7 (BA):about 0.9-1.1 (OL); or about 4.6 (OA):about 4.4 (UA):about 0.6 (BA):about 1 (OL).

In some embodiments, the molar ratios of the individual triterpenes in the triterpene mixture is in the range of about 15.6 OA to about 4 UA to about 1 BA, or about 16 OA to about 4 UA to about 1 BA, or in the range of about 15-16 OA to about 3.5-4.5 UA to about 0.5-1.5 BA, or in the range of about 15.4-15.8 OA to about 3.8-4.2 UA to about 0.8-1.2 BA.

In some embodiments, the molar ratio of the OA:UA is about 4 OA to about 1 UA, the molar ratio OA:UA:BA is about P:Q:1 or greater, wherein P is at least 4, and Q is at least 1, (e.g. about 4:1:1 or greater, about 8:2:1 or greater, or about 16:4:1 or greater), and the molar of OA+UA:BA is about 5:1 or greater (or about 10:1 or greater, or about 20:1 or greater). Exemplary acceptable molar ratios of OA:UA:BA include about 4:1:1, about 8:2:1, about 16:4:1, about 32:8:1, about 64:16:1, about 128:32:1, about 256:64:1.

In some embodiments, the molar ratio of UA:BA is about (0.04-0.8):1, the molar ratio of OA:UA:BA is about X:(0.04-0.8):1 or greater, wherein X is about 0.04 or greater. Exemplary acceptable molar ratios of OA:UA:BA include about 0.04:0.04:1, about 0.08:0.04:1, about 0.12:0.04.1, about 0.15:0.04:1, about 0.31:0.04:1, about 0.62:0.04:1, about 1.24:0.04:1, about 2.5:0.04:1, about 0.04:0.08:1, about 0.08:0.08:1, about 0.12:0.08.1, about 0.15:0.08:1, about 0.31:0.08:1, about 0.62:0.08:1, about 1.24:0.08:1, about 2.5:0.08:1, or greater.

Oleandrin is optionally present in any triterpene composition of the invention.

In some embodiments, a composition comprising oleandrin (OL) and one or more components extractable from Nerium species plant material is more efficacious than pure oleandrin, when equivalent doses based upon oleandrin content are compared. In some embodiments, the molar ratio of oleandrin to said one or more components extractable from Nerium species plant mass in the range of about 100:1 to about 1:100.

Embodiments of the invention include those wherein the plant material (biomass) is obtained from Nerium sp., Nerium oleander, Nerium oleander L (Apocynaceae), Nerium odourum, white oleander, pink oleander, Agrobacterium tumefaciens (Ibrahim et al., “Stimulation of oleandrin production by combined Agrobacterium tumefaciens mediated transformation and fungal elicitation in Nerium oleander cell cultures” in Enz. Microbial Technol. (2007), 41(3), 331-336, the entire disclosure of which is hereby incorporated by reference), or a combination thereof. In some embodiments, the biomass comprises leaves, stems, flowers, bark, fruits, seeds, sap, and/or pods. Nerium oleander can be obtained from microculture in vitro, whereby shoot cultures can be initiated from seedlings and/or from shoot apices of the Nerium oleander cultivars Splendens giganteum, Revanche or Alsace, or other cultivars (Vila et al., “Micropropagation of Oleander (Nerium oleander L.)” in HortScience (2010), 45(1), 98-102, the entire disclosure of which is hereby incorporated by reference). Nerium oleander plant material can be obtained, for example, from commercial plant suppliers such as Aldridge Nursery, Atascosa, Tex.

In some embodiments, a composition comprising one or more components, excluding oleandrin, which are extractable from plant material is more efficacious than pure oleandrin, when equivalent doses based upon oleandrin content are compared.

In some embodiments, the other therapeutic agent or the one or more other components is not a polysaccharide obtained during preparation of the extract, meaning it is not an acidic homopolygalacturonan or arabinogalaturonan. In some embodiments, the other therapeutic agent or the one or more other components is a polysaccharide obtained during preparation of the extract, meaning it is an acidic homopolygalacturonan or arabinogalaturonan.

In some embodiments, the extract excludes another therapeutic agent and/or excludes an acidic homopolygalacturonan or arabinogalaturonan obtained during preparation of the extract. In some embodiments, the extract comprises or further comprises another therapeutic agent and/or an acidic homopolygalacturonan or arabinogalaturonan obtained during preparation of the extract.

The invention also provides use of oleandrin in the manufacture of a medicament for the treatment of GM in a subject. In some embodiments, the manufacture of such a medicament comprises: providing one or more compounds of the invention; including a dose of said compound(s) in a pharmaceutical dosage form; and packaging the pharmaceutical dosage form. In some embodiments, the manufacture can be conducted as described in PCT International Application No. PCT/US06/29061. The manufacture can also include one or more additional steps such as: delivering the packaged dosage form to a vendor (retailer, wholesaler and/or distributor); selling or otherwise providing the packaged dosage form to a subject having GM; including with the medicament a label and a package insert, which provides instructions on use, dosing regimen, administration, content and toxicology profile of the dosage form.

In some embodiments, the treatment of GM comprises: determining that a subject has GM; indicating administration of pharmaceutical dosage form to the subject according to a dosing regimen; administering to the subject one or more pharmaceutical dosage forms, wherein the one or more pharmaceutical dosage forms is administered according to the dosing regimen.

The pharmaceutical composition can further comprise a combination of at least one material selected from the group consisting of a water soluble (miscible) co-solvent, a water insoluble (immiscible) co-solvent, a surfactant, an antioxidant, a chelating agent, and an absorption enhancer.

The solubilizer is at least a single surfactant, but it can also be a combination of materials such as a combination of: a) surfactant and water miscible solvent; b) surfactant and water immiscible solvent; c) surfactant, antioxidant; d) surfactant, antioxidant, and water miscible solvent; e) surfactant, antioxidant, and water immiscible solvent; f) surfactant, water miscible solvent, and water immiscible solvent; or g) surfactant, antioxidant, water miscible solvent, and water immiscible solvent.

The pharmaceutical composition optionally further comprises a) at least one liquid carrier; b) at least one emulsifying agent; c) at least one solubilizing agent; d) at least one dispersing agent; e) at least one other excipient; or f) a combination thereof.

In some embodiments, the water miscible solvent is low molecular weight (less than 6000) PEG, glycol, or alcohol. In some embodiments, the surfactant is a pegylated surfactant, meaning a surfactant comprising a poly(ethylene glycol) functional group.

The invention includes all combinations of the aspects, embodiments and sub-embodiments of the invention disclosed herein.

### BRIEF DESCRIPTION OF THE FIGURES

The following figures form part of the present description and describe exemplary embodiments of the claimed invention. The skilled artisan will, in light of these figures and the description herein, be able to practice the invention without undue experimentation.

FIG. 1A depicts the radiotherapy/pharmacotherapy combination protocol of Example 5 for evaluation of the peroral PBI-05204 and X-ray irradiation for treating GBM orthotopically injected into mice.

FIG. 1B depicts a chart of survival time for mice with orthotopically-injected human GBM cells, which were subjected to treatment with PBI-05204, XRT (X-ray radiotherapy), and the combination of PBI-05204 and XRT according to Example 5.

FIG. 2A depicts the dosing used for the combination protocol including radiotherapy (XRT; single dose (4 Gy) at day-10 after initiation of treatment), chemotherapy (32 mg TMZ/Kg bodyweight for three consecutive days starting at day-9 after initiation of treatment), and pharmacotherapy (peroral 40 mg PBI-05204/Kg bodyweight/day; five consecutive days per week for five weeks). FIGS. 2B-2D depict charts of survival time for mice with orthotopically-injected U87 GBM cells (into the brain tissue of the mice), which were subjected to treatment with TMZ, PBI-05204, XRT, XRT and PBI-05204, TMZ and PBI-05204, and the combination of TMZ+XRT+PBI-05204 according to Example 6.

FIGS. 3A-3C depict cell cycle analysis charts for U87MG cells treated with PBI-05204 and measured by PI and ANNEXIN-V staining according to Example 14. Red arrow indicates Annexin V positive cells.

FIGS. 4A-4C depict charts establishing the dose response of PBI-05204 against human GBM U87MG, U251, and T98 cells by apoptosis as determined by caspase 3, 8, and 9 enzymatic activity. Data are presented as Mean+/−SD ** p<0.01, *** p<0.001, p<0.0001 versus control.

FIGS. 5A-5C depict charts establishing the antiproliferative activity of PBI-05204 agains established human GBM U87MG, U251, and T98 cells based upon a crystal violet assay showing the growth of cells was inhibited by PBI-05204 treatment evidenced by the changes in staining of U87MG, U251, and T98G cell lines, morphological changes of different doses of PBI-05204 in U87MG cells, and growth curve (FIGS. 5A-5C).

FIGS. 6A-6C depict photographs of Western blot gels establishing the impact of PBI-05204 upon cell growth and apoptotic as well as cell signaling pathways in human GBM cells as established by down-regulating PI3K/mTOR pathways in human GBM cell lines. FIG. 6A depicts Western blots indicating protein expression of Beclin 1, NFκB p65, and Gadd45b in U87MG cells after treatment with PBI-05204. FIG. 6B depicts Western blots indicating Protein expression of Beclin 1, NF-κB p65, and Gadd45b in U251 cells after treatment with PBI-05204. FIG. 6C depicts Western blots for p-Akt (Ser473), P-Akt (Thr308), Ser235/236 p-S6 and Ser65 p-4E-BP1 proteins in EGF prestimulated U87MG cells after being treated with PBI-05204 for 72 hrs.

FIGS. 7A and 7B depict charts establishing the dose response of PBI-05204 against human GBM U87MG, U251, and T98 cells as determined by p-Akt (Ser473) and p-mTOR (Ser 2448) enzymatic activity. Data are presented as Mean+/−SD ** p<0.01, *** p<0.001, **** p<0.0001 versus control.

FIG. 8 depicts a chart quantifying and establishing decreased spheroid formation in GSC (glioma stem cells) after treatment with PBI-05204.

FIGS. 9A-9C depict charts quantifying and establishing decreased protein expression of CD44, CXCR4 and Sox2 in U87 cells after being treated with PBI-05204 for 24 hrs. Data are presented as Mean±SD. * p<0.05, ** p<0.01, *** p<0.001, **** p<0.0001 versus control.

FIG. 10 depicts a schematic of the treatment protocol employed in Example 17 wherein PBI-05204 is administered mice bearing human GBM subcutaneous xenograft mouse tumor (U87MG, U251, T98G).

FIGS. 11A and 11B depict the tumor growth curve (FIG. 11A) and weight (FIG. 11B) of U87MG xenograft of FIG. 10.

FIGS. 12A and 12B depict the tumor growth curve (FIG. 12A) and tumor weight (FIG. 12B) of U251 xenograft of FIG. 10.

FIGS. 13A and 13B depict the tumor growth curve (FIG. 13A) and weight (FIG. 13B) of T98G xenograft of FIG. 10.

FIG. 14 depicts a schematic of the treatment protocol employed in Example 18 wherein PBI-05204 is administered (10 mg/Kg) mice bearing human GBM tumor U87MG-luc cells in a mouse orthotopic intra-brain model.

FIG. 15A depicts a chart quantifying the disease-free survival (DFS) for the mice of FIG. 14. FIG. 15B depicts a chart quantifying the overall survival (OS) for the mice of FIG. 14. Data are presented as Mean±SD. A) p<0.05 versus control; b) p<0.01 versus control; c) p<0.05 versus PBI-05204.

FIGS. 16A-16B depict graphs showing the dose response of the GBM cells lines U87MG (FIG. 16A) and T98G (FIG. 16B) to treatment with a triterpene mixture versus control (DMSO). The concentration of the individual triterpenes (present at a OA:UA:BA molar ratio of about 16:4:1) in the solutions was as follows: “2×OUB” denotes 12.5 microM OA, 3.16 microM UA, 0.8 microM BA; “1×OUB” denotes 6.25 microM OA, 1.58 microM UA, 0.4 microM BA; and “4×OUB” denotes 25 microM OA, 6.32 microM UA, 1.6 microM BA.

FIGS. 17A-17E depict charts showing the efficacy of the triterpenes OA, UA, and BA individually and in different combinations against the GBM stem cell lines GBM9 or GS28. “CTG-FA_mean” denotes the mean inhibition of growth of GBM stem cells relative to control as determined by cell-titer glow assay. 1.00 denotes 100% inhibition of cell growth. FIGS. 17A-17C and 17E depict the results for evaluation against the GBM9 stem cell line. FIG. 17D depicts the results for evaluation against the GS28 stem cell line.

### DETAILED DESCRIPTION OF THE INVENTION

The invention provides a pharmacotherapy method of treating GM, especially GBM, in a subject by chronic administration of a composition, pharmaceutical composition, or oleandrin-containing composition (OCC) as described herein during a treatment period. The pharmacotherapy method can be combined with at least one of radiotherapy, chemotherapy, and surgery, and said combining can be sequential and/or in overlapping manner.

The composition is administered according to a dosing regimen best suited for the subject, the suitability of the dose and dosing regimen to be determined clinically according to conventional clinical practices and clinical treatment endpoints.

As used herein, the term “subject” is taken to mean any living being, creature or animal. In some embodiments, subject is taken to mean warm-blooded animal or cold-blooded animal. Exemplary warm-blooded animals include mammals, for example, cats, dogs, mice, rats, guinea pigs, horses, bovine cows, sheep, and humans.

As used herein, the term glioblastoma includes initial (newly discovered) and recurrent (recurring) gliobastoma. Unless otherwise specified GM may be used herein interchangeably with GBM.

A subject treated according to the invention will exhibit a therapeutic response. By “therapeutic response” is meant that a subject suffering from GM will enjoy at least one of the following clinical benefits as a result of treatment with a cardiac glycoside: eradication of GM the subject's brain, amelioration of GM, reduction in the occurrence of symptoms associated with GM, partial or full remission of GM, increased time to progression of GM, reduced rate of progression of GM, increased overall survival (in a population of subjects with GM), increase glioma-free survival time, and/or increased disease-free survival time. The therapeutic response can be a full or partial therapeutic response.

As used herein, “time to progression” is the period, length or duration of time after GM is diagnosed (or treated) until the GM begins to worsen. It is the period of time during which the size (severity) of the GM is maintained without further progression of the GM, and the period of time ends when the GM begins to progress again. Progression of a disease is determined by “staging” a subject suffering from the GM prior to or at initiation of therapy. For example, the subject's health is determined prior to or at initiation of therapy. The subject is then treated with OCC, and the progression of GM is monitored periodically. At some later point in time, the symptoms of the GM may worsen, thus marking progression of the GM and the end of the “time to progression”. The period of time during which the GM did not progress or during which the level or severity of the infection did not worsen is the “time to progression”.

As used herein, the term “treatment period” is taken to mean the period of time beginning with the initial treatment of a subject having GM and ending with the final treatment of said subject. The treatment period begins with the first of initiation of resection, iniation of radiotherapy, iniation of chemotherapy, or iniation of pharmacotherapy. The treatment period ends with the last of completion of radiotherapy, completion of chemotherapy, or completion of pharmacotherapy. In some embodiments, the treatment period begins with initiation of a combination protocol and ends with completion of a combination protocol. The treatment period can be one or more weeks, one or more months, one or more quarters and/or one or more years.

As used herein, the terms “resistant GM” (rGM) or “treatment resistant GM” (trGM) are used interchangeably and refer to glioma that has been treated by radiotherapy and/or chemotherapy but which nonetheless has not resulted in a full remission in a subject. The GM remains in the subject and continues to progress. The same is true for “resistant GBM” (rGBM) and “treatment resistant GBM” (trGBM).

As used herein, the terms “recurrent GM” (rGM) or “recurrent GBM” (rGBM) refer to a GM, or GBM respectively, that develops in a subject after the subject has already experienced a remission of a prior GM, or GBM respectively.

As used herein, the term “cardiac glycoside” includes at least oleandrin.

A dosing regimen includes a therapeutically relevant dose (or effective dose) of one or more cardiac glycosides (e.g. at least oleandrin) administered according to a dosing schedule. A therapeutically relevant dose, therefore, is a therapeutic dose at which a therapeutic response of the GM to treatment with composition, pharmaceutical composition, or OCC (oleandrin-containing composition) is observed and at which a subject can be administered the composition, pharmaceutical composition, or OCC without an excessive amount of unwanted or deleterious side effects. A therapeutically relevant dose is non-lethal to a subject, even though it may cause some side effects (adverse events) in the patient. It is a dose at which the level of clinical benefit to a subject being administered the composition, pharmaceutical composition, or OCC exceeds the level of deleterious side effects experienced by the subject due to administration of the composition, pharmaceutical composition, or OCC or component(s) thereof. A therapeutically relevant dose will vary from subject to subject according to a variety of established pharmacologic, pharmacodynamic and pharmacokinetic principles. However, a therapeutically relevant dose (relative, for example, to oleandrin) will typically be about about 25 micrograms, about 100 micrograms, about 250 micrograms, about 500 micrograms or about 750 micrograms of cardiac glycoside/day or it can be in the range of about 25-750 micrograms of cardiac glycoside per dose, or might not exceed about 25 micrograms, about 100 micrograms, about 250 micrograms, about 500 micrograms or about 750 micrograms of oleandrin/day. Another example of a therapeutically relevant dose (relative, for example, to triterpene either individually or together) will typically be in the range of about 0.1 micrograms to 100 micrograms, about 0.1 mg to about 500 mg, about 100 to about 1000 mg per kg of body weight, about 15 to about 25 mg/kg, about 25 to about 50 mg/kg, about 50 to about 100 mg/kg, about 100 to about 200 mg/kg, about 200 to about 500 mg/kg, about 10 to about 750 mg/kg, about 16 to about 640 mg/kg, about 15 to about 750 mg/kg, about 15 to about 700 mg/kg, or about 15 to about 650 mg/kg of bodyweight, i.e. mg of oleandrin per bodyweight. It is known in the art that the actual amount of OCC required to provide a target therapeutic result in a subject may vary from subject to subject according to the basic principles of pharmacy.

A dose of radiation administered to a subject generally refers to the “absorbed dose”, which is the fundamental quantity for describing the effects of radiation in a tissue or organ. Absorbed dose is the energy deposited in a small volume of matter (tissue) by the radiation beam passing through the matter divided by the mass of the matter. Absorbed dose is thus measured in terms of energy deposited per unit mass of material. Absorbed dose is measured in joules/kilogram, and a quantity of 1 joule/kilogram has the special unit of gray (Gy) in the International System of quantities and units. In terms of the older system of radiation quantities and units previously used, 1 Gy equals 100 rad, or 1 mGy equals 0.1 rad. The biological effects of an absorbed dose of a given magnitude are dependent on the type of radiation delivering the energy (i.e., whether the radiation is from x rays, gamma rays, electrons (beta rays), alpha particles, neutrons, or other particulate radiation) and the amount of radiation absorbed. This variation in effect is due to the differences in the manner in which the different types of radiation interact with tissue. The variation in the magnitude of the biological effects due to different types of radiation is described by the “radiation weighting factor” for the specific radiation type. The radiation weighting factor is a dimensionless constant, the value of which depends on the type of radiation. Thus, the absorbed dose (in Gy) averaged over an entire organ and multiplied by a dimensionless factor, the radiation weighting factor, gives the equivalent dose. The unit for the quantity equivalent dose is the sievert (Sv). Thus, the relation is as follows:

equivalent dose (in Sv)=absorbed dose (in Gy)×radiation weighting factor.

In the older system of units, equivalent dose was described by the unit rem and 1 Sv equals 100 rem or 1 mSv equals 0.1 rem.

A GM or GBM may be removed by resection. Surgical resection of the tumor is the current standard of care. In some embodiments, surgical resection is conducted prior to radiotherapy, prior to chemotherapy, and/or prior to pharmacotherapy with a composition of the invention. In some embodiments, surgical resection is conducted after radiotherapy, after chemotherapy, and/or after pharmacotherapy with a composition of the invention. In preferred embodiments, resection of the GM is conducted before radiotherapy, before chemotherapy, and before pharmacotherapy with a composition, pharmaceutical composition, or OCC of the invention

Generally, a more extensive surgical resection is associated with longer life expectancy, achieving the longest survival in those patients who undergo gross total resection followed by radiotherapy and chemotherapy. In some embodiments, the margins of the tumors are visually enhanced with a fluorescent agent prior to resection of the tumor.

Chemotherapy generally refers to administration of an anticancer compound to a subject with GM or GBM. Suitable anticancer compounds include nitrosoureas, DNA alkylating agent(s), temozolomide (TMZ), carmustine (BCNU), lomustine (CCNU), nimustine (ACNU), fotemusine, cediranib, erlotinib, galunisertib, irinotecan, procarbazine, vincristine, bevacizumab, hydroxyureas, and cytarabine. In some embodiments, the anticancer compound is temozolomide. A current standard of care for patients with nGBM is maximum safe surgical resection followed by concurrent TMZ (75 mg/m²/day for 6 weeks) and RT (60 Gy in 30 fractions) and then six maintenance cycles of TMZ (150-200 mg/m2/day for the first 5 days of a 28-day cycle—sdTMZ), according to the results of the phase III EORTC 26981.

A therapeutically relevant dose can be administered according to any dosing regimen typically used in the treatment of GM. A therapeutically relevant dose can be administered once, twice, thrice or more daily. It can be administered every other day, every third day, every fourth day, every fifth day, semiweekly, weekly, biweekly, every three weeks, every four weeks, monthly, bimonthly, semimonthly, every three months, every four months, semiannually, annually, or according to a combination of any of the above to arrive at a suitable dosing schedule. For example, a therapeutically relevant dose can be administered one or more times daily (up to 10 times daily for the highest dose) for one or more weeks.

The invention provides a method of treating GM in a mammal or host cell, the method comprising: administering a composition, pharmaceutical composition, or OCC to the mammal or host cell.

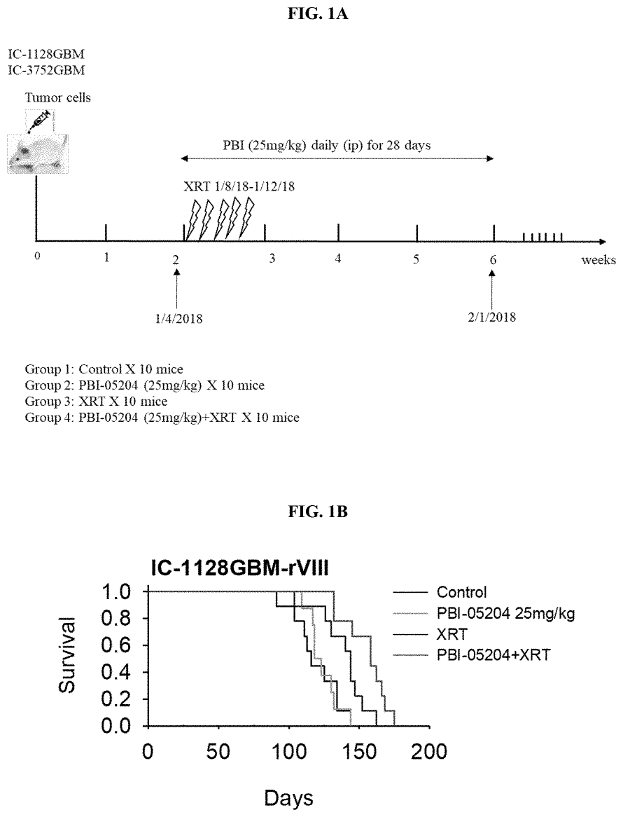

Example 5 provides a detailed description of a combination protocol used to used to evaluate the efficacy of OCC comprising PBI-05204 (supercritical fluid (SCF) extract of Nerium oleander, said SCF extract comprising oleandrin and one or more other active ingredients extractable from said Nerium oleander) in combination with radiotherapy but excluding chemotherapy and resection of the GM according to the protocol set forth in FIG. 1A.

Tumor cells were orthotopically injected with GM tumor cells (IC1128GBM, IC3752GBM). After a two-week of tumor development, fractionated X-ray therapy (XRT, for five days) and pharmacotherapy (chronic administration of PBI-05204; 25 mg/Kg; i.p. daily for 28 days) was initiated. The mice were divided into four groups (ten mice per group): Group 1: control—received no radiotherapy or pharmacotherapy; Group 2: received PBI-05204; Group 3: received XRT; and Group 4: received XRT and PBI-05204. The results (FIG. 1B); indicate the following order in terms of overall survival (in the group) over time: Group 4>Group 2>Group 3>Group 1 (control).

Accordingly, the invention provides a combination protocol method of treating GM, esp. GBM, in a subject, said method comprising: subjecting said subject to fractionated X-ray radiotherapy for a period of at least about 5 days; and chronically administering a composition, pharmaceutical composition, or OCC to said subject on a daily basis for a period of at least about 28 days. The fractionated total dose of radiation is typically evenly divided over said days. The total dose of composition, pharmaceutical composition, or OCC is typically evenly divided over said days.

Example 6 provides a detailed description of a combination protocol used to used to evaluate the efficacy of OCC comprising PBI-05204 (supercritical fluid (SCF) extract of Nerium oleander, said SCF extract comprising oleandrin and one or more other active ingredients extractable from said Nerium oleander) in combination with radiotherapy and chemotherapy but excluding resection of the GM according to the protocol set forth in FIG. 1A.

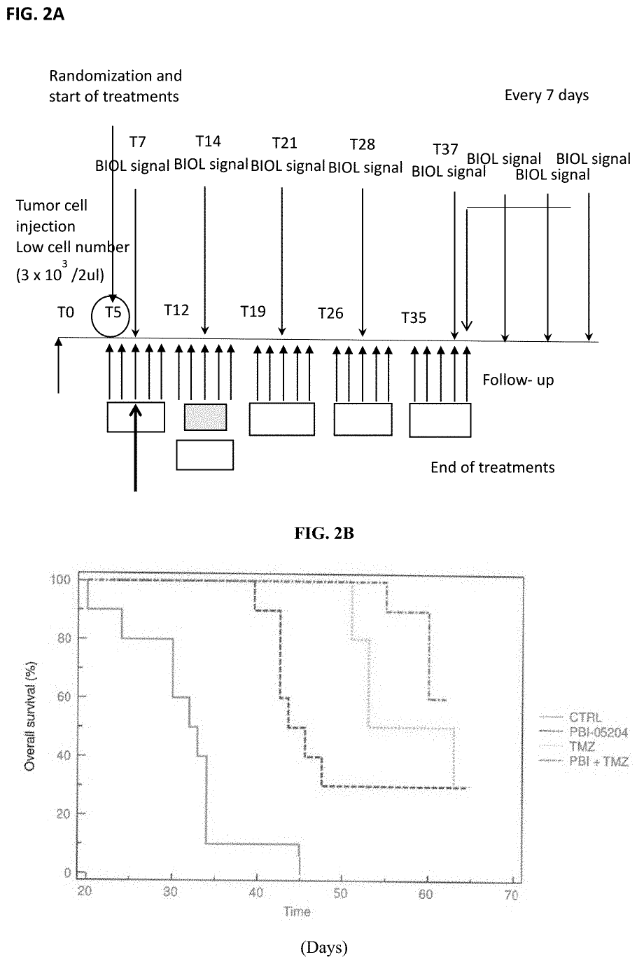

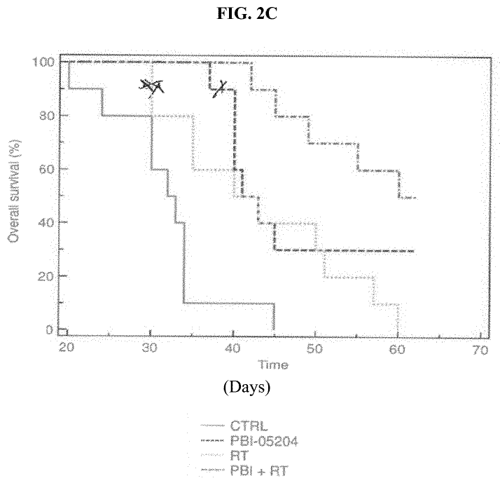

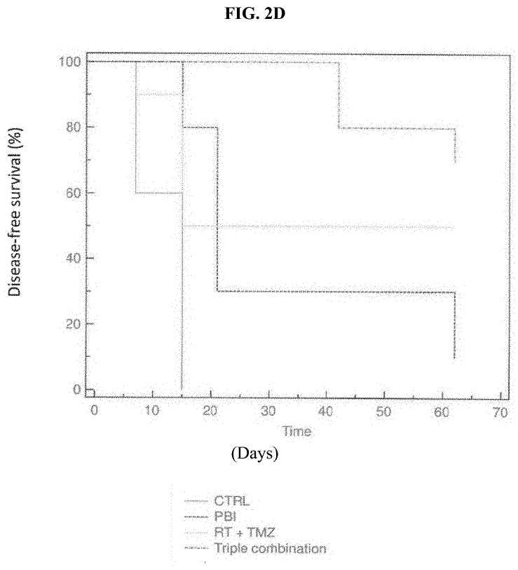

Tumor cells were orthotopically injected with GM tumor cells (U87GBM; 3×10³ cell/2 microL) into the brain tissue. After five days of tumor development, pharmacotherapy (chronic administration of PBI-05204; 40 mg/Kg; p.o. five days per week for five weeks) was initiated. TMZ was administered (32 mg TMZ/Kg bodyweight) was administered four three days starting on day 9 after initiation of pharmacotherapy. A single dose (4 Gy) of X-ray radiotherapy was administered on day 10 after initiation of pharmacotherapy. The mice were divided into 8 groups: Group 1: control—received only vehicle and no XRT, TMZ, or PBI-05204; Group 2: received TMZ and no XRT or PBI-05204; Group 3: received PBI-05204 in vehicle and no XRT or TMZ; Group 4: received XRT and no TMZ or PBI-05204; Group 5: received XRT and PBI-05204 and no TMZ; Group 6: received PBI-05204 and TMZ; Group 7: received XRT and TMZ; and Group 8: received XRT, TMZ, and PBI-05204. The results (FIGS. 2B-2D) indicate the following order in terms of overall survival (in the group) over time: FIG. 2B: Group 6>Group 2>Group 3>Group 1 (control); FIG. 2C: Group 5>Group 3>Group 4>Group 1 (control); FIG. 2D: Group 8>Group 7>Group 3>Group 1 (control).

Accordingly, the invention provides a combination protocol method of treating GM, esp. GBM, in a subject, said method comprising: chronically administering a composition, pharmaceutical composition, or OCC on a daily basis to said subject five days per week for at least five weeks; treating said subject to at least a dose of X-ray radiation; and treating said subject to at least three doses of TMZ. The total dose of TMZ is typically evenly divided over said days. The total dose of composition, pharmaceutical composition, or OCC is typically evenly divided over said days.

The invention also provides a method of treating GM, in particular GBM, in a subject, the method comprising chronically administering to said subject composition, pharmaceutical composition, or OCC, irradiating the GM of said subject with X-ray radiation at least once, and administering at least a dose of TMZ to said subject without resecting the GM from said subject.

The invention also provides a method of treating GM, in particular GBM, in a subject, the method comprising chronically administering composition, pharmaceutical composition, or OCC to said subject, irradiating the GM of said subject with X-ray radiation at least once, and administering at least a dose of TMZ to said subject, and resecting the GM from said subject prior to or after said administering composition, pharmaceutical composition, or OCC.

The invention also provides a method of treating GM, in particular GBM, in a subject, the method comprising chronically administering composition, pharmaceutical composition, or OCC to said subject, irradiating the GM of said subject with X-ray radiation wherein the total dose of radiation is fractionated over two or more days, and administering plural doses of TMZ to said subject. Said method can include or exclude resection of the GM.

The invention also provides a method of treating GM, in particular GBM, in a subject, the method comprising administering plural doses of composition, pharmaceutical composition, or OCC to said subject, irradiating the GM of said subject with plural doses of X-ray radiation, and administering plural doses of TMZ to said subject. Said method can include or exclude resection of the GM.

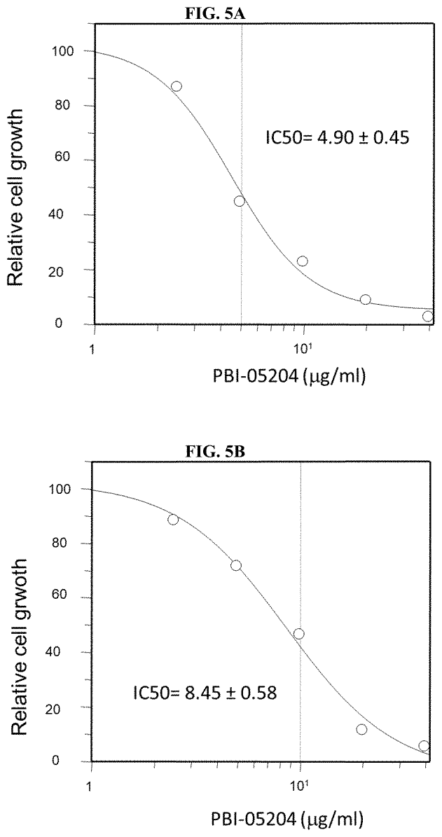

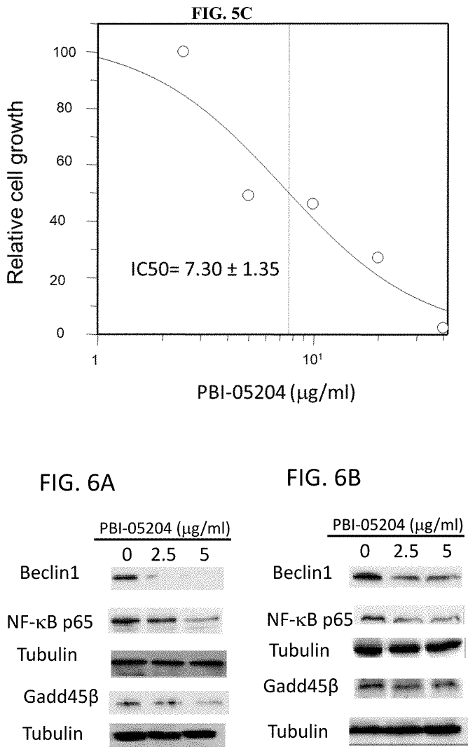

The in vitro GBM cell line response was determined using U87MG, U251 and T98g cell lines. All three human GBM cell lines responded to PBI-05204 with a concentration dependent inhibition of proliferation as shown in FIGS. 5A-5C. IC₅₀ values calculated for established GBM cells and stem like glioma cells (GSCs) were similar and ranged between 0.5 and 10 μg/ml. IC₅₀ values in all three GBM cells were comparable and ranged from 4.9 to 8.45 ug/ml when cells were treated with PBI-05204 for 72 hr. PBI-05204 treated U87MG cells had an elongated cell morphology with concentration dependent increased numbers of vacuoles. The increased aberrant cell morphology and decreased cell numbers were noted at the highest concentrations of PBI-05204 treated U87MG. U251 and T98G cell lines behaved in a similar manner when exposed to drug. Morphologic changes were associated with a concentration-dependent increase in drug mediated apoptosis as evidenced by PI and Annexin V staining via flow cytometry as well as increased caspase activities.

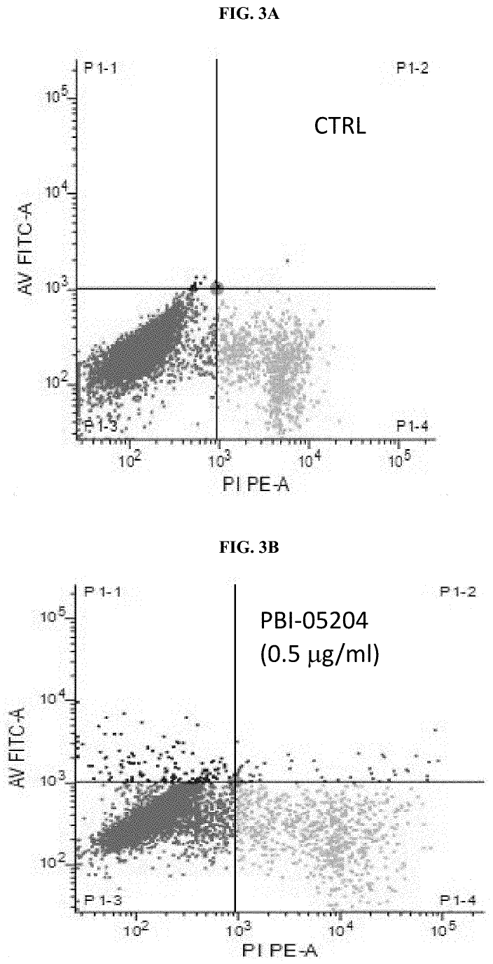

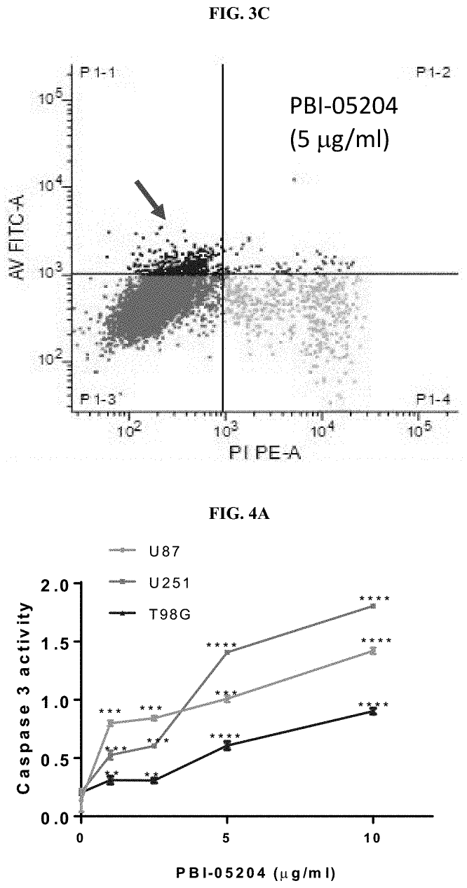

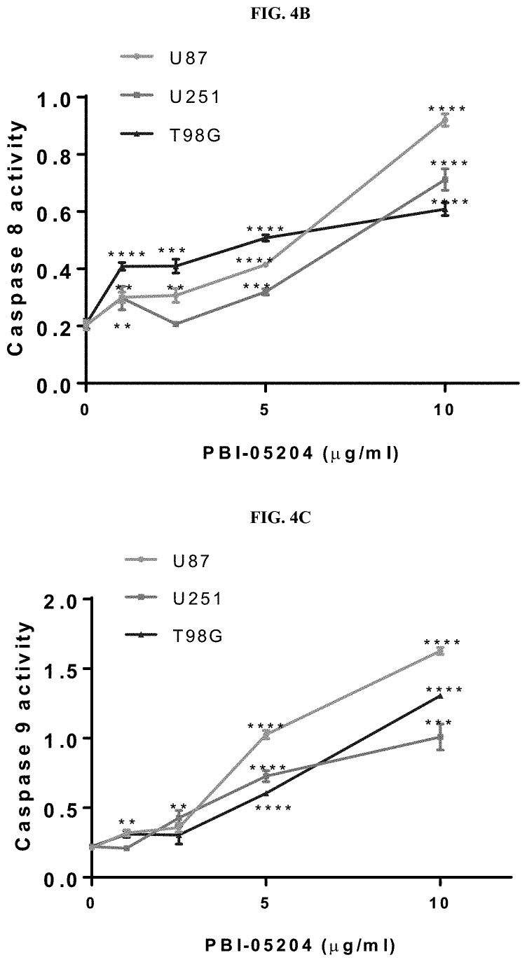

A cell cycle analysis (Example 14) was conducted to determine the impact of PBI-05204 upon apoptotic death of GM cells. The data (FIGS. 3A-3C) indicate there was an increase in Annexin V positive cells in PBI-05204 treated U87MG cells compared to that of vehicle control group. The red arrow in FIG. 3C indicates the apoptotic cell population. The PBI-05204 induced apoptotic cells death in GBM cells was also evidenced by increases in caspase 3, 8 and 9 activities (FIGS. 4A-4C). The pan-caspase inhibitor, z-VAD-fmk, largely inhibited PBI-05204 induced caspase 3 activity. In addition, caspase-3 was activated.

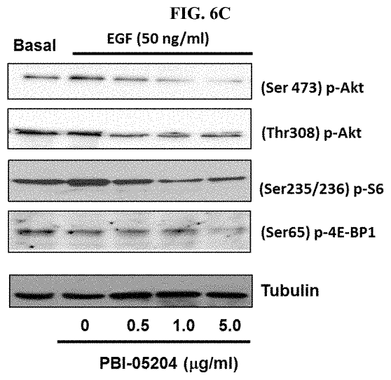

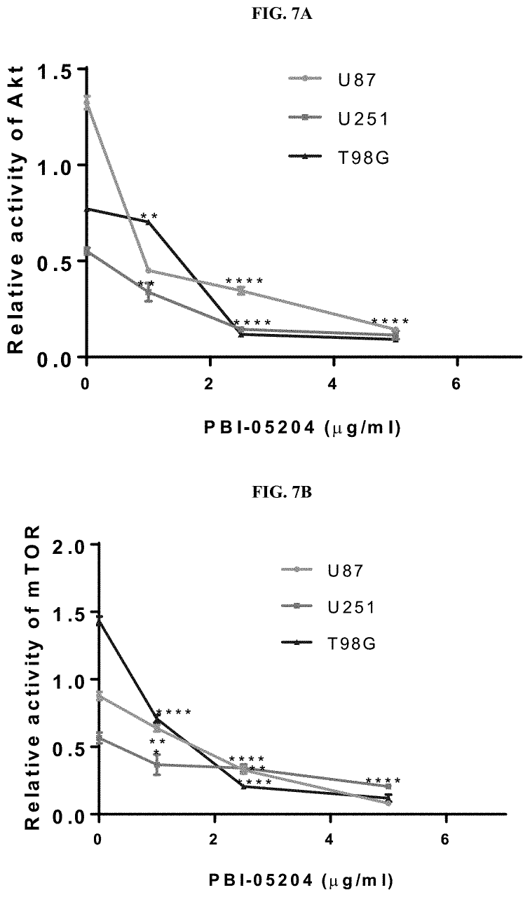

The level of enzymatic activity for key enzymes was determined. Evidence of PBI-05204 mediated ability to induce apoptosis was shown with Western blot analyses (FIGS. 6A-6C) of U87MG and U251 cells in which concentration dependent declines in expression of Beclin1, NF-kβ p65 and Gadd45p were evident in both cell lines. A clear indication of drug mediated inhibition of the p-Akt pathway was also observed due to reduced expression of Ser473 and Thr308 sites of Akt phosphorylation in addition to declines in expression of Ser235/236 p-S6 and Ser56 p-4E-BP1. PBI-05204 treatment of all three GBM cell lines demonstrated (FIGS. 7A, 7B) a concentration-dependent inhibition of both Akt and mTOR pathway activities which are commonly elevated in GBM.

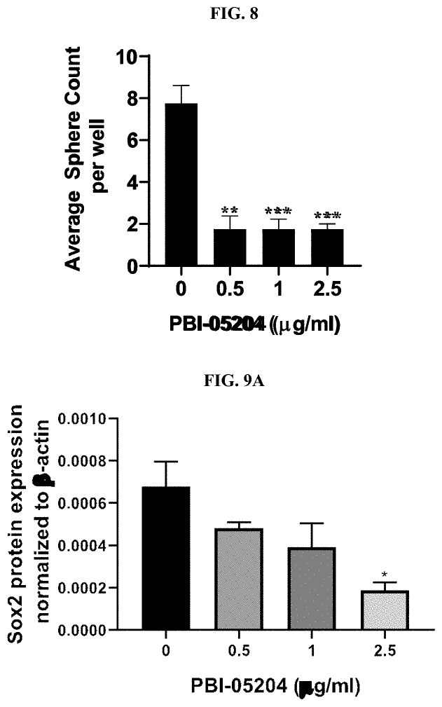

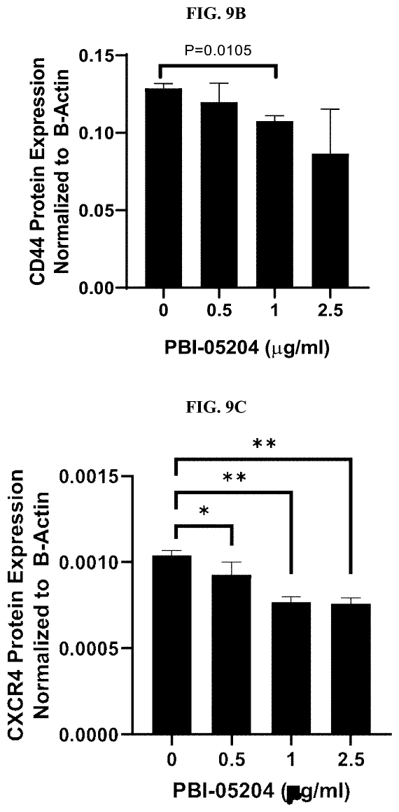

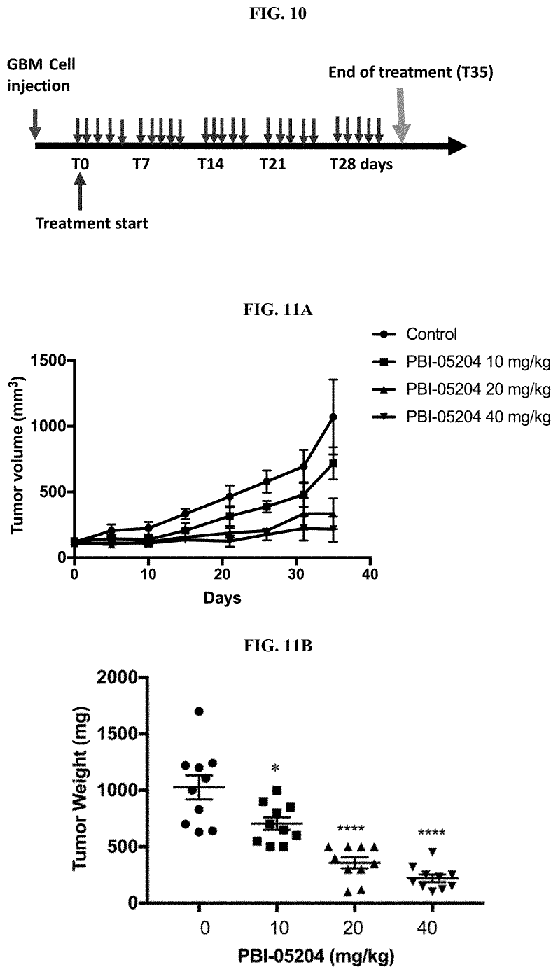

Drug mediated inhibition of tumor cell line proliferation alone, while important, might be considered by some to be insufficient to suggest a new and effective treatment for GBM unless a drug can be shown to be effective against GBM stem cells that work to regenerate and maintain tumor growth after initial therapy. Treatment of U87MG tumor spheroids was therefore examined and specific stem cell markers were evaluated. The data (FIG. 8) indicate that drug treatment of U87MG cells grown as tumor spheroids led to a significant decrease in spheroid size and number. Moreover, we show the amount and size of newly formed neurospheres and the level of neural stem cell markers, such as CXCR4, CD44 and SOX2, are decreased in the presence of PBI-05204 thereby reducing self-renewal and stemness of GSCs (FIGS. 9A-9C). Analyses of the PBI-05204 treated spheroids revealed declines in expression of tumor stem cell markers CD44, CXCR4 and SOX2 in U87 cells. Both the number and size of single spheres of patient derived GBM stem cells (BT48EF) were also reduced due to exposure to PBI-05204. Drug treatment of these cells resulted in a significant percentage of stem like cells induced to adhere to the plastic which was considered as a pre-requisite for glioma stem cell differentiation to the perineural/neural phenotype associated with reduced stem cell markers and increased differentiation markers (data not shown).

The ability of a composition to reduce the size and/or number of stem cell spheroids is indicative of its efficacy against recurrent or treatment resistant GM (or GBM). This ability is rare, if not unique, in the field of GM (or GBM) treatments. Prior to this work, it was not known or contemplated that the composition(s) of the invention could be used to treat recurrent or treatment resistant GM (or GBM).

Accordingly, the invention provides a method of treating recurrent or treatment resistant GM (or GBM) comprising chronically administering to a subject in need therapeutically effective dose(s) of a composition as described herein, whereby reducing the number or size of spheroids of GM (or GBM) stem cells in the subject.

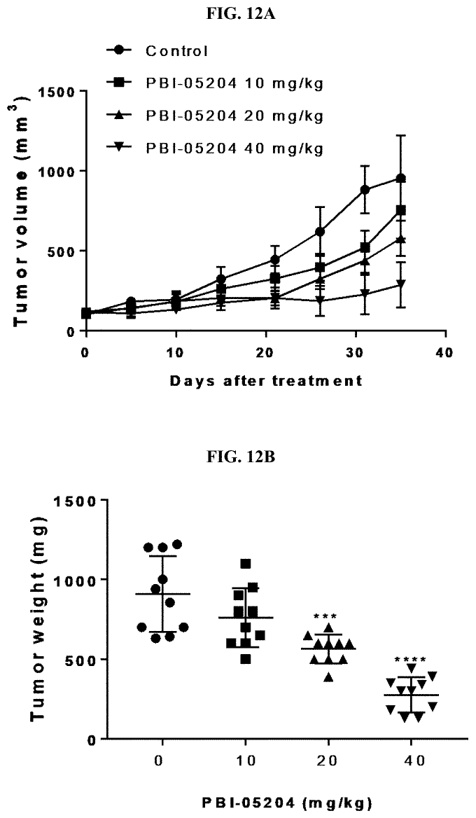

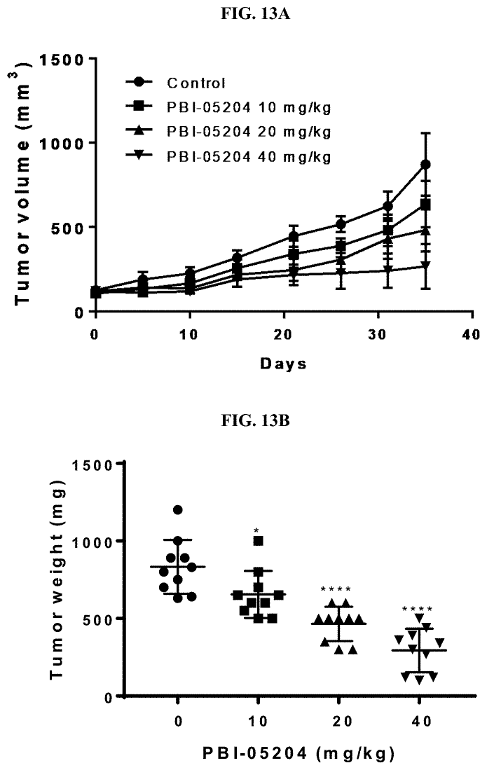

Additional evidence of the in vivo efficacy of PBI-05204 against GBM cells was obtained using a human GBM subcutaneous xenograft mouse model using the protocol of FIG. 10. GBM cells growing as subcutaneous tumors responded well to oral doses of PBI-05204. Mice which received 40 mg/kg PBI-05204 demonstrated very little tumor growth at all over the 35-day treatment period. Both the 20 mg/kg and 40 mg/kg dose levels resulted in significant inhibition of excised tumor weight compared to non-treated animals. This was evident for all three GBM cell lines tested as xenografts. Specifically, doses of 10, 20 or 40 mg/kg PBI-05204 produced 31.3%, 65.2% and 78.5% reductions respectively in excised day 35 tumor weight compared to untreated control mice with U87MG tumors (FIGS. 11A-11B). In mice with U251 tumors these dose dependent reductions were 16.1%, 37.7% and 69.5% (FIGS. 12A-12B) while in mice with T98G tumors the excised tumors showed reduction in weights of 21.4%, 44.2% and 64.7% (FIGS. 13A-13B).

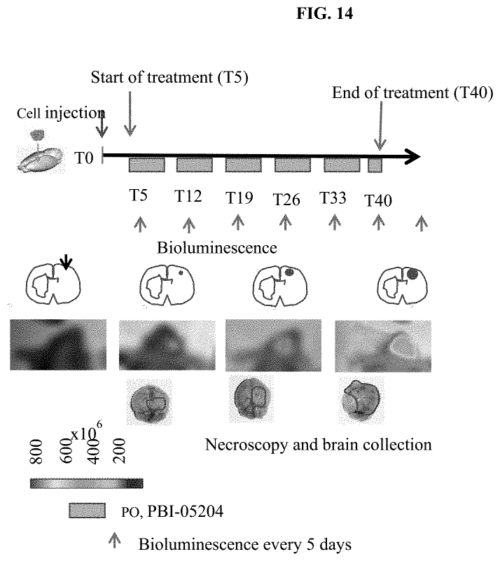

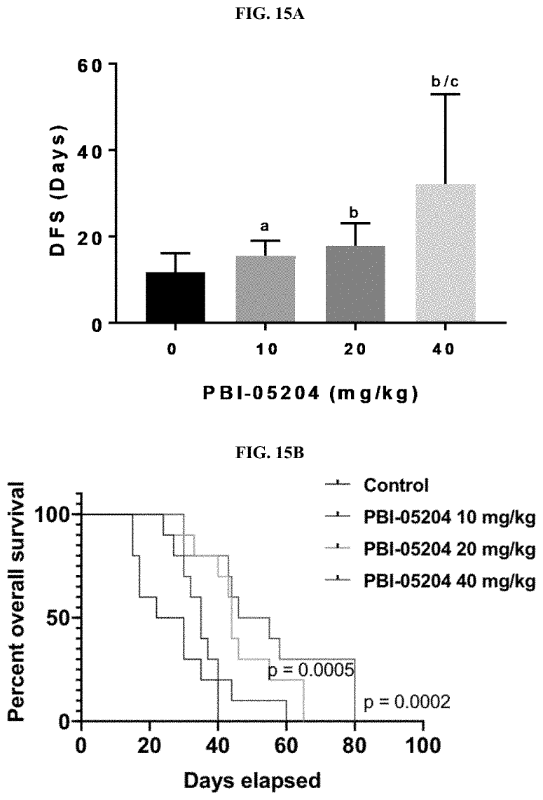

Additional evidence of the in vivo efficacy of PBI-05204 against GBM cells was obtained using a human GBM orthotropic intra-brain mouse model using the protocol of FIG. 14. Mice were injected with a small number of U87-luc tumor cells (3×10³). Mice received PBI-05204 orally over 35 days with a 45 day non-drug follow up period. Tumor growth in mice was measured every five days over a 40-day period of time as shown in FIG. 14. The highest dose of PBI-05204 tested resulted in 50% survival of 55 days versus untreated mice which a 50% group survival of only 28 days. As shown in FIGS. 15A-15B, the time necessary to detect bioluminescence in intra-brain tumors increased after PBI-0524 administration in a dose-dependent manner. Control mice developed a bioluminescent lesion after 11.80±4.13 days. The mean day of bioluminescence appearance (DFS) was 15.60±3.44 (p=0.0383 vs. the control), 17.90±5.15 (p=0.0091 vs control) and 32.10±20.77 (P=0.0072 vs CTRL) in mice treated with 10, 20 and 40 mg/kg/day, respectively. Overall survival (OS) increased after PBI-0524 administration in a dose-dependent manner. OS in control mice was 26.60±10.47 days. In contrast, values of OS increased in drug treated mice with 36.40±10.49 days (p=0.0480 vs. the control), 45.90±12.95 (p=0.0019 vs control) and 54.60±19.66 days (p=0.0014 vs control) for 10, 20 and 40 mg/kg/day groups, respectively.

GBM brain tissue obtained from the mice of Example 18 was analyzed after completion of the dosing. Excised brain tissues from the GBM xenograft mice were examined for expression of well characterized tumor and stem cell markers. Ki67 was significantly reduced following administration of PBI-05204 at all doses tested suggesting a strong and significant inhibition of tumor cell proliferation and growth. TUNEL, an established marker of apoptotic DNA fragmentation, was elevated in tumor tissues showing a dose dependent effect with respect to increased apoptosis. The decline in expression of CD31 demonstrates microvasculature within tumor tissue was decreased by PBI-05204. Tissues were also subjected to Western blot examination of total Akt protein expression as well as pAKT^{ser473} and pAKT^{Thr308} normalized to total Akt. There was a clear dose dependent inhibition of expression of both phosphorylated forms of Akt indicating inhibition of activation of this important pathway in GBM tumor tissue from mice administered PBI-05204.

The invention thus provides a method of treating GM, in particular GBM, in a subject, the method comprising administering to said subject composition, pharmaceutical composition, or OCC without resecting the GM from said subject.

The invention also provides a method of treating GM, in particular GBM, in a subject, the method comprising administering to said subject composition, pharmaceutical composition, or OCC prior to or after resecting the GM from said subject.

PBI-05204 (as described herein and in U.S. Pat. No. 8,187,644 B2 to Addington, which issued May 29, 2012, U.S. Pat. No. 7,402,325 B2 to Addington, which issued Jul. 22, 2008, U.S. Pat. No. 8,394,434 B2 to Addington et al, which issued Mar. 12, 2013, the entire disclosures of which are hereby incorporated by reference) comprises cardiac glycoside (oleandrin, OL) and triterpenes (oleanolic acid (OA), ursolic acid (UA) and betulinic acid (BA)) as the primary pharmacologically active components. The molar ratio of OL to total triterpene is about 1:(10-96). The molar ratio of OA:UA:BA is about 7.8:7.4:1. The combination of OA, UA and BA in PBI-05204 might increase the anticancer activity of oleandrin when compared on an OL equimolar basis. PBI-04711 is a fraction of PBI-05204, but it does not contain cardiac glycoside (OL). The molar ratio of OA:UA:BA in PBI-04711 is about 3:2.2:1. PBI-04711 might also possesses anticancer activity. Accordingly, an OCC comprising OL, OA, UA, and BA may be more efficacious than a composition comprising OL as the sole active ingredient based upon an equimolar content of OL. In some embodiments, the molar ratios of the individual triterpenes to oleandrin range as follows: about 2-8 (OA):about 2-8 (UA):about 0.1-1 (BA):about 0.5-1.5 (OL); or about 3-6 (OA):about 3-6 (UA):about 0.3-8 (BA):about 0.7-1.2 (OL); or about 4-5 (OA):about 4-5 (UA):about 0.4-0.7 (BA):about 0.9-1.1 (OL); or about 4.6 (OA):about 4.4 (UA):about 0.6 (BA):about 1 (OL).

Compositions, pharmaceutical compositions, or OCC's comprising oleandrin as the sole active agent are within the scope of the invention.

Compositions, pharmaceutical compositions, or OCC's comprising oleandrin and plural triterpenes as the active agents are within the scope of the invention. In some embodiments, the compositions, pharmaceutical composition, or OCC comprises oleandrin, oleanolic acid (free acid, salt, derivative or prodrug thereof), ursolic acid (free acid, salt, derivative or prodrug thereof), and betulinic acid (free acid, salt, derivative or prodrug thereof). The molar ratios of the compounds are as described herein.

Also within the scope of the invention are use of Compositions, pharmaceutical compositions, or OCC's comprising oleandrin and at least one or more active ingredients selected from the group consisting of cardiac glycoside, glycone, aglycone, steroid, triterpene, polysaccharide, saccharide, alkaloid, fat, protein, neritaloside, odoroside, oleanolic acid, ursolic acid, betulinic acid, oleandrigenin, oleaside A, betulin (urs-12-ene-3β,28-diol), 28-norurs-12-en-3β-ol, urs-12-en-3β-ol, 3β,3β-hydroxy-12-oleanen-28-oic acid, 3β,20α-dihydroxyurs-21-en-28-oic acid, 3β,27-dihydroxy-12-ursen-28-oic acid, 3β,13β-dihydroxyurs-11-en-28-oic acid, 3β,12α-dihydroxyoleanan-28,13β-olide, 3β,27-dihydroxy-12-oleanan-28-oic acid, homopolygalacturonan, arabinogalaturonan, chlorogenic acid, caffeic acid, L-quinic acid, 4-coumaroyl-CoA, 3-O-caffeoylquinic acid, 5-O-caffeoylquinic acid, cardenolide B-1, cardenolide B-2, oleagenin, neridiginoside, nerizoside, odoroside-H, 3-beta-O-(D-diginosyl)-5-beta, 14 beta-dihydroxy-card-20(22)-enolide pectic polysaccharide composed of galacturonic acid, rhamnose, arabinose, xylose, and galactose, polysaccharide with MW in the range of 17000-120000 D, or MW about 35000 D, about 3000 D, about 5500 D, or about 12000 D, cardenolide monoglycoside, cardenolide N-1, cardenolide N-2, cardenolide N-3, cardenolide N-4, pregnane, 4,6-diene-3,12,20-trione, 20R-hydroxypregna-4,6-diene-3,12-dione, 16beta,17beta-epoxy-12beta-hydroxypregna-4,6-diene-3,20-dione, 12beta-hydroxypregna-4,6,16-triene-3,20-dione (neridienone A), 20S,21-dihydroxypregna-4,6-diene-3,12-dione (neridienone B), neriucoumaric acid, isoneriucoumaric acid, oleanderoic acid, oleanderen, 8alpha-methoxylabdan-18-oic acid, 12-ursene, kaneroside, neriumoside, 3β-O-(D-diginosyl)-2α-hydroxy-8,14β-epoxy-5β-carda-16:17,20:22-dienolide, 3β-O-(D-diginosyl)-2α,14β-dihydroxy-5β-carda-16:17,20:22-dienolide, 3β,27-dihydroxy-urs-18-en-13,28-olide, 3β,22α,28-trihydroxy-25-nor-lup-1(10),20(29)-dien-2-one, cis-karenin (3β-hydroxy-28-Z-p-coumaroyloxy-urs-12-en-27-oic acid), trans-karenin (3-β-hydroxy-28-E-p-coumaroyloxy-urs-12-en-27-oic acid), 3beta-hydroxy-5alpha-carda-14(15),20(22)-dienolide (beta-anhydroepidigitoxigenin), 3 beta-O-(D-digitalosyl)-21-hydroxy-5beta-carda-8,14,16,20(22)-tetraenolide (neriumogenin-A-3beta-D-digitaloside), proceragenin, neridienone A, 3beta,27-dihydroxy-12-ursen-28-oic acid, 3beta,13beta-dihydroxyurs-11-en-28-oic acid, 3beta-hydroxyurs-12-en-28-aldehyde, 28-orurs-12-en-3beta-ol, urs-12-en-3beta-ol, urs-12-ene-3beta,28-diol, 3beta,27-dihydroxy-12-oleanen-28-oic acid, (20S,24R)-epoxydammarane-3beta,25-diol, 20beta,28-epoxy-28alpha-methoxytaraxasteran-3beta-ol, 20beta,28-epoxytaraxaster-21-en-3beta-ol, 28-nor-urs-12-ene-3beta,17 beta-diol, 3beta-hydroxyurs-12-en-28-aldehyde, alpha-neriursate, beta-neriursate, 3alpha-acetophenoxy-urs-12-en-28-oic acid, 3beta-acetophenoxy-urs-12-en-28-oic acid, oleanderolic acid, kanerodione, 3β-p-hydroxyphenoxy-11α-methoxy-12α-hydroxy-20-ursen-28-oic acid, 28-hydroxy-20(29)-lupen-3,7-dione, kanerocin, 3alpha-hydroxy-urs-18,20-dien-28-oic acid, D-sarmentose, D-diginose, neridiginoside, nerizoside, isoricinoleic acid, gentiobiosylnerigoside, gentiobiosylbeaumontoside, gentiobiosyloleandrin, folinerin, 12β-hydroxy-5β-carda-8,14,16,20(22)-tetraenolide, 8β-hydroxy-digitoxigenin, Δ16-8β-hydroxy-digitoxigenin, Δ16-neriagenin, uvaol, ursolic aldehyde, 27(p-coumaroyloxy)ursolic acid, oleanderol, 16-anhydro-deacteyl-nerigoside, 9-D-hydroxy-cis-12-octadecanoic acid, adigoside, adynerin, alpha-amyrin, beta-sitosterol, campestrol, caoutchouc, capric acid, caprylic acid, choline, cornerin, cortenerin, deacetyloleandrin, diacetyl-nerigoside, foliandrin, pseudocuramine, quercetin, quercetin-3-rhamnoglucoside, quercitrin, rosaginin, rutin, stearic acid, stigmasterol, strospeside, urehitoxin, and uzarigenin, and any combination thereof.

Also within the scope of the invention are use of compositions or pharmaceutical compositions, comprising or further comprising at least one or more active ingredients selected from the group consisting of cardiac glycoside, glycone, aglycone, steroid, triterpene, polysaccharide, saccharide, alkaloid, fat, protein, neritaloside, odoroside, oleanolic acid, ursolic acid, betulinic acid, oleandrigenin, oleaside A, betulin (urs-12-ene-3β,28-diol), 28-norurs-12-en-3β-ol, urs-12-en-3β-ol, 3β,3β-hydroxy-12-oleanen-28-oic acid, 3β,20α-dihydroxyurs-21-en-28-oic acid, 3β,27-dihydroxy-12-ursen-28-oic acid, 3β,13β-dihydroxyurs-11-en-28-oic acid, 3β,12α-dihydroxyoleanan-28,13β-olide, 3β,27-dihydroxy-12-oleanan-28-oic acid, homopolygalacturonan, arabinogalaturonan, chlorogenic acid, caffeic acid, L-quinic acid, 4-coumaroyl-CoA, 3-O-caffeoylquinic acid, 5-O-caffeoylquinic acid, cardenolide B-1, cardenolide B-2, oleagenin, neridiginoside, nerizoside, odoroside-H, 3-beta-O-(D-diginosyl)-5-beta, 14 beta-dihydroxy-card-20(22)-enolide pectic polysaccharide composed of galacturonic acid, rhamnose, arabinose, xylose, and galactose, polysaccharide with MW in the range of 17000-120000 D, or MW about 35000 D, about 3000 D, about 5500 D, or about 12000 D, cardenolide monoglycoside, cardenolide N-1, cardenolide N-2, cardenolide N-3, cardenolide N-4, pregnane, 4,6-diene-3,12,20-trione, 20R-hydroxypregna-4,6-diene-3,12-dione, 16beta,17beta-epoxy-12beta-hydroxypregna-4,6-diene-3,20-dione, 12beta-hydroxypregna-4,6,16-triene-3,20-dione (neridienone A), 20S,21-dihydroxypregna-4,6-diene-3,12-dione (neridienone B), neriucoumaric acid, isoneriucoumaric acid, oleanderoic acid, oleanderen, 8alpha-methoxylabdan-18-oic acid, 12-ursene, kaneroside, neriumoside, 3β-O-(D-diginosyl)-2α-hydroxy-8,14β-epoxy-5β-carda-16:17,20:22-dienolide, 3β-O-(D-diginosyl)-2α,14β-dihydroxy-5β-carda-16:17,20:22-dienolide, 3β,27-dihydroxy-urs-18-en-13,28-olide, 3β,22α,28-trihydroxy-25-nor-lup-1(10),20(29)-dien-2-one, cis-karenin (3β-hydroxy-28-Z-p-coumaroyloxy-urs-12-en-27-oic acid), trans-karenin (3-β-hydroxy-28-E-p-coumaroyloxy-urs-12-en-27-oic acid), 3beta-hydroxy-5alpha-carda-14(15),20(22)-dienolide (beta-anhydroepidigitoxigenin), 3 beta-O-(D-digitalosyl)-21-hydroxy-5beta-carda-8,14,16,20(22)-tetraenolide (neriumogenin-A-3beta-D-digitaloside), proceragenin, neridienone A, 3beta,27-dihydroxy-12-ursen-28-oic acid, 3beta,13beta-dihydroxyurs-11-en-28-oic acid, 3beta-hydroxyurs-12-en-28-aldehyde, 28-orurs-12-en-3beta-ol, urs-12-en-3beta-ol, urs-12-ene-3beta,28-diol, 3beta,27-dihydroxy-12-oleanen-28-oic acid, (20S,24R)-epoxydammarane-3beta,25-diol, 20beta,28-epoxy-28alpha-methoxytaraxasteran-3beta-ol, 20beta,28-epoxytaraxaster-21-en-3beta-ol, 28-nor-urs-12-ene-3beta,17 beta-diol, 3beta-hydroxyurs-12-en-28-aldehyde, alpha-neriursate, beta-neriursate, 3alpha-acetophenoxy-urs-12-en-28-oic acid, 3beta-acetophenoxy-urs-12-en-28-oic acid, oleanderolic acid, kanerodione, 3β-p-hydroxyphenoxy-11α-methoxy-12α-hydroxy-20-ursen-28-oic acid, 28-hydroxy-20(29)-lupen-3,7-dione, kanerocin, 3alpha-hydroxy-urs-18,20-dien-28-oic acid, D-sarmentose, D-diginose, neridiginoside, nerizoside, isoricinoleic acid, gentiobiosylnerigoside, gentiobiosylbeaumontoside, gentiobiosyloleandrin, folinerin, 12β-hydroxy-5β-carda-8,14,16,20(22)-tetraenolide, 8β-hydroxy-digitoxigenin, Δ16-8β-hydroxy-digitoxigenin, Δ16-neriagenin, uvaol, ursolic aldehyde, 27(p-coumaroyloxy)ursolic acid, oleanderol, 16-anhydro-deacteyl-nerigoside, 9-D-hydroxy-cis-12-octadecanoic acid, adigoside, adynerin, alpha-amyrin, beta-sitosterol, campestrol, caoutchouc, capric acid, caprylic acid, choline, cornerin, cortenerin, deacetyloleandrin, diacetyl-nerigoside, foliandrin, pseudocuramine, quercetin, quercetin-3-rhamnoglucoside, quercitrin, rosaginin, rutin, stearic acid, stigmasterol, strospeside, urehitoxin, uzarigenin and any combination thereof. Additional components that may be present in the extract are disclosed by Gupta et al. (IJPSR (2010), 1(3), 21-27, the entire disclosure of which is hereby incorporated by reference).

PBI-01011 is an improved triterpene-based composition comprising OA, UA and BA, wherein the molar ratio of OA:UA:BA is about 9-12:up to about 2:up to about 2, or about 10:about 1:about 1, or about 9-12:about 0.1-2:about 0.1-2, or about 9-11:about 0.5-1.5:about 0.5-1.5, or about 9.5-10.5:about 0.75-1.25:about 0.75-1.25, or about 9.5-10.5:about 0.8-1.2:about 0.8-1.2, or about 9.75-10.5:about 0.9-1.1:about 0.9-1.1.

The triterpenes used herein may be present as free acid, salt, prodrug, or derivative forms. Their molar ratios and amounts are relative to the respective parent triterpene free acid.

Compositions comprising triterpenes and excluding oleandrin were evaluated for their ability to treat recurrent or treatment resistant GBM, said activity being correlated with the composition's ability to reduce the size and/or number of spheroids of GBM stem cells. The compositions comprised the individual triterpenes or various dual or triple combinations thereof.

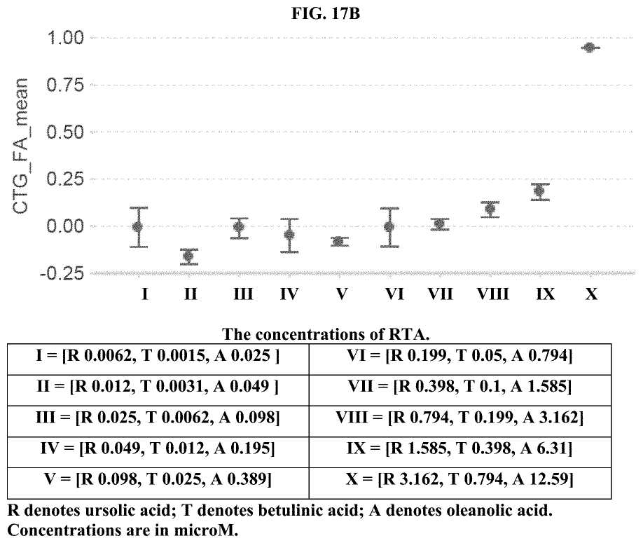

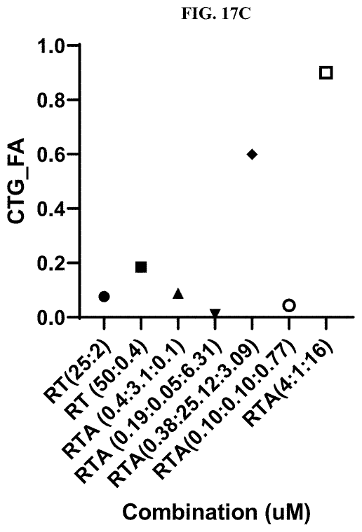

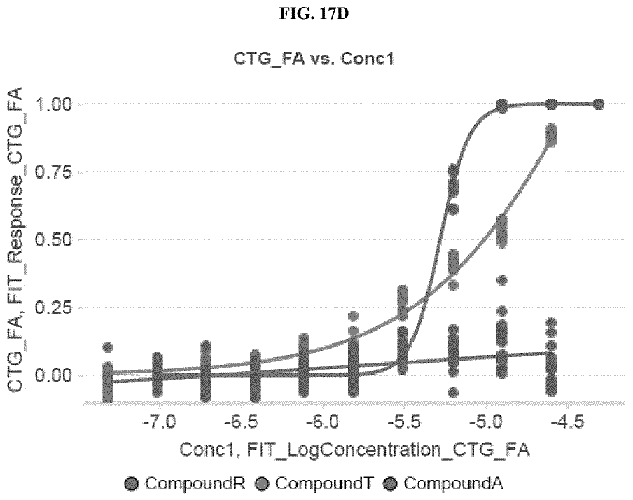

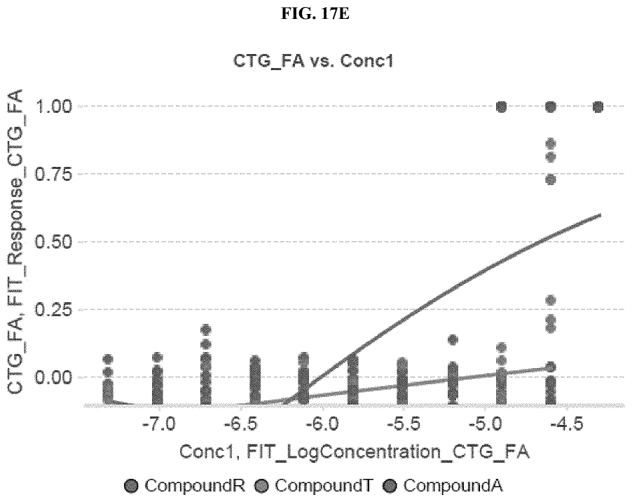

The results depicted in FIG. 17D were obtained by evaluating the dose dependent efficacy of the individual triterpenes ursolic acid (compound R), betulinic acid (compound T), and oleanolic acid (compound A) against the GS28 stem cell line. The results indicated that oleanolic acid exhibits very poor activity at all concentrations ranging from 1×10^{-4.5} to 1×10⁻⁷ M. Ursolic acid and betulinic acid exhibit activity at concentrations greater than about 5.6 microM (greater than 1×10^{-5.25}) but little to no activity at concentrations below that.

The results depicted in FIG. 17E were obtained by evaluating the dose dependent efficacy of the individual triterpenes ursolic acid (compound R), betulinic acid (compound T), and oleanolic acid (compound A) against the GBM9 stem cell line. The results indicated that oleanolic acid exhibits very poor activity at all concentrations ranging from 1×10^{-4.5} to 1×10⁻⁷ M. Ursolic acid and betulinic acid exhibit activity at concentrations greater than about 10 microM (greater than 1×10⁻⁵) but little to no activity at concentrations below that.

Over one thousand samples containing varying concentrations of the individual, dual combination, and triple combination triterpenes were then evaluated against the GBM9 stem cell line according to the bliss assay described below. It was found that a vast majority of the samples exhibited little to no activity toward inhibition of growth of GBM9 stem cell spheroids. FIGS. 17A and 17C depict some of the results, which are summarized in the following table.

| FIG. 17A | | FIG. 17C | |
|---|---|---|---|
| | ART (OUB) | Sample order: | ART (OUB) |
| Sample color | ratio | left to right | ratio |
| Blue, purple, | About 16:4:1 | First | 12.5:1 |
| yellow, green, | | | |
| orange | | | |
| Pink, brown | 20:5:1 | Second | 125:1 |
| Red, grey | 16.7:3.33:1 | Third | 0.03:0.13:1 |
| Black | 10:3:1 | Fourth | 126.2:3.8:1 |
| | | Fifth | 0.12:0.015:1 |
| | | Sixth | 7.7:1:1 |
| | | Seventh | About 16:4:1 |

In each case, the sample having the molar ratio of about 16:4:1 ART (OUB) exhibits unexpectedly high activity. This is especially surprising, because oleanolic acid on its own exhibited little to no activity at all concentrations tested.

The data further demonstrated that triterpene compositions exhibited significant efficacy at inhibiting stem cell spheroid growth when the composition was defined as follows: molar ratio of the OA:UA is about 4 OA to about 1 UA, the molar ratio OA:UA:BA is about P:Q:1 or greater, wherein P is at least 4, and Q is at least 1, (e.g. about 4:1:1 or greater, about 8:2:1 or greater, or about 16:4:1 or greater), and the molar of OA+UA:BA is about 5:1 or greater (or about 10:1 or greater, or about 20:1 or greater). Exemplary acceptable molar ratios of OA:UA:BA include about 4:1:1, about 8:2:1, about 16:4:1, about 32:8:1, about 64:16:1, about 128:32:1, about 256:64:1.

The data further demonstrated that triterpene compositions also exhibited significant efficacy at inhibiting stem cell spheroid growth when the composition was defined as follows: the molar ratio of UA:BA is about (0.04-0.8):1, the molar ratio of OA:UA:BA is about X:(0.04-0.8):1 or greater, wherein X is about 0.04 or greater. Exemplary acceptable molar ratios of OA:UA:BA include about 0.04:0.04:1, about 0.08:0.04:1, about 0.12:0.04.1, about 0.15:0.04:1, about 0.31:0.04:1, about 0.62:0.04:1, about 1.24:0.04:1, about 2.5:0.04:1, about 0.04:0.08:1, about 0.08:0.08:1, about 0.12:0.08.1, about 0.15:0.08:1, about 0.31:0.08:1, about 0.62:0.08:1, about 1.24:0.08:1, about 2.5:0.08:1, or greater.

The dose response of the 16:4:1 OUB combination (herein referred to as PBI-01641) was then evaluated against the GBM9 stem cell line. The results are depicted in FIG. 17B. Even though the relative molar ratio of the three triterpenes was kept constant, the data demonstrated a substantial difference between the sample IX (R=about 3.16 microM, T=about 0.79 microM, and A=about 12.6 microM) and sample X (R=about 1.59 microM, T=about 0.4 microM, and A=about 6.3 microM). Samples I through VIII exhibited little in vitro activity. The dose dependent response for the concentrations between sample IX and X was further evaluated while keeping the molar ratio constant.

PBI-01641 is an improved triterpene-based composition comprising OA, UA, and BA, wherein the molar ratio of OA:UA:BA is about 15-17:about 3-5:about 0.5-1.5; or about 15.5-16.5:about 3.5-4.5:about 0.75-1.25; or about 16:about 4:about 1.

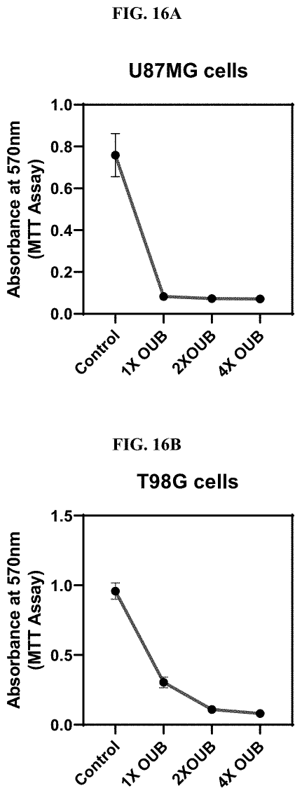

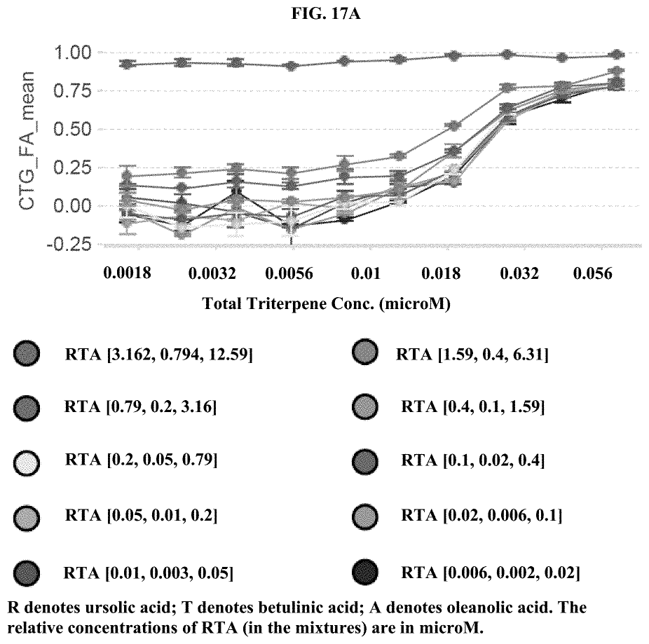

The efficacy of the PBI-01641 composition was evaluated against the U87MG (FIG. 16A) and T98G (FIG. 16B) GBM cell lines (“2×OUB” denotes 12.5 microM OA, 3.16 microM UA, 0.8 microM BA; “1×OUB” denotes 6.25 microM OA, 1.58 microM UA, 0.4 microM BA; and “4×OUB” denotes 25 microM OA, 6.32 microM UA, 1.6 microM BA). The results of these assays indicate whether the composition will be active against first time or initial GBM in a subject, because these assays do not focus on activity against growth of the stem cell spheroids. The results indicated that the composition also exhibits strong efficacy even at lower concentrations when used to treat GBM cell lines that are not treatment resistant.

Accordingly, the invention also provides a method of treating recurrent or treatment resistant GM (or GBM) comprising chronically administering to a subject in need thereof a pharmaceutical composition comprising a) oleandrin; b) an extract comprising oleandrin; c) an extract of Nerium species; d) one or more components of an extract of Nerium species; or e) a mixture of at least three triterpenes (oleanolic acid (OA), ursolic acid (UA), betulinic acid (BA)) present at a molar ratio of about 15-17 OA:about 3-5 UA:about 0.5-1.5 BA; or about 15.5-16.5 OA:about 3.5-4.5 UA:about 0.75-1.25 BA; or about 16 OA:about 4 UA:about 1 BA.

The invention also provides a method of treating recurrent or treatment resistant GM (or GBM) comprising chronically administering to a subject in need thereof a pharmaceutical composition comprising a mixture of at least three triterpenes (oleanolic acid (OA), ursolic acid (UA), betulinic acid (BA)), wherein the molar ratio of OA:UA is about 4 OA to about 1 UA, the molar ratio OA:UA:BA is about P:Q:1 or greater, wherein P is at least 4, and Q is at least 1, (e.g. about 4:1:1 or greater, about 8:2:1 or greater, or about 16:4:1 or greater), and the molar of OA+UA:BA is about 5:1 or greater (or about 10:1 or greater, or about 20:1 or greater). Exemplary acceptable molar ratios of OA:UA:BA include about 4:1:1, about 8:2:1, about 16:4:1, about 32:8:1, about 64:16:1, about 128:32:1, about 256:64:1.

The invention also provides a method of treating recurrent or treatment resistant GM (or GBM) comprising chronically administering to a subject in need thereof a pharmaceutical composition comprising a mixture of at least three triterpenes (oleanolic acid (OA), ursolic acid (UA), betulinic acid (BA)), wherein the molar ratio of OA:UA:BA is about X:(0.04-0.8):1 or greater, wherein X is about 0.04 or greater. Exemplary acceptable molar ratios of OA:UA:BA include about 0.04:0.04:1, about 0.08:0.04:1, about 0.12:0.04.1, about 0.15:0.04:1, about 0.31:0.04:1, about 0.62:0.04:1, about 1.24:0.04:1, about 2.5:0.04:1, about 0.04:0.08:1, about 0.08:0.08:1, about 0.12:0.08.1, about 0.15:0.08:1, about 0.31:0.08:1, about 0.62:0.08:1, about 1.24:0.08:1, about 2.5:0.08:1, or greater

In some embodiments, the OCC comprises oleandrin, at least oleanolic acid (free acid, salt, derivative or prodrug thereof) and ursolic acid (free acid, salt, derivative or prodrug thereof) present at a molar ratio of OA to UA as described herein. OA is present in large molar excess over UA.

In some embodiments, the OCC comprises oleandrin, at least oleanolic acid (free acid, salt, derivative or prodrug thereof) and betulinic acid (free acid, salt, derivative or prodrug thereof) present at a molar ratio of OA to BA as described herein. OA is present in large molar excess over BA.

In some embodiments, the OCC comprises oleandrin, at least oleanolic acid (free acid, salt, derivative or prodrug thereof), ursolic acid (free acid, salt, derivative or prodrug thereof), and betulinic acid (free acid, salt, derivative or prodrug thereof) present at a molar ratio of OA to UA to BA as described herein. OA is present in large molar excess over both UA and BA.

In general, a subject having GM, e.g. GBM, is treated as follows. The subject is evaluated to determine whether said subject has a GM. Administration of OCC is indicated. Initial doses of OCC are administered to the subject according to a prescribed dosing regimen for a period of time (a treatment period). The subject's clinical response and level of therapeutic response are determined periodically. If the level of therapeutic response is too low at one dose, then the dose is escalated according to a predetermine dose escalation schedule until the desired level of therapeutic response in the subject is achieved. Treatment of the subject with OCC is continued as needed. The dose or dosing regimen can be adjusted as needed until the patient reaches the desired clinical endpoint(s).

If a clinician intends to treat a subject having GM with a combination of oleandring and one or more other therapeutic agents, and it is known that the GM is at least partially therapeutically responsive to treatment with said one or more other therapeutic agents, then the present method invention comprises: administering to the subject in need thereof a therapeutically relevant dose of OCC and a therapeutically relevant dose of said one or more other therapeutic agents, wherein the OCC is administered according to a first dosing regimen and the one or more other therapeutic agents is administered according to a second dosing regimen. In some embodiments, the first and second dosing regimens are the same. In some embodiments, the first and second dosing regimens are different.

The composition, pharmaceutical, or OCC of the invention can be administered as primary pharmacotherapy, adjunct pharmacotherapy, or co-pharmacotherapy. Methods of the invention include separate administration or coadministration of the composition, pharmaceutical, or OCC with at least one other known composition, meaning the composition, pharmaceutical, or OCC can be administered before, during or after administration of the known composition (compound(s)) or of a composition for treating symptoms associated with GM. For example, medications used to treat vomiting, nausea, headache, pain, confusion, memory loss, difficulty with balance, urinary incontinence, vision problems, personality changes, irritability, or edema can be administered with or separately from the composition, pharmaceutical, or OCC.

The one or more other therapeutic agents can be administered at doses and according to dosing regimens that are clinician-recognized as being therapeutically effective or at doses that are clinician-recognized as being sub-therapeutically effective. The clinical benefit and/or therapeutic effect provided by administration of a combination of composition, pharmaceutical, or OCC and one or more other therapeutic can be additive or synergistic, such level of benefit or effect being determined by comparison of administration of the combination to administration of the individual OCC component(s) and one or more other therapeutic agents. The one or more other therapeutic agents can be administered at doses and according to dosing regimens as suggested or described by the Food and Drug Administration, World Health Organization, European Medicines Agency (E.M.E.A.), Therapeutic Goods Administration (TGA, Australia), Pan American Health Organization (PAHO), Medicines and Medical Devices Safety Authority (Medsafe, New Zealand) or the various Ministries of Health worldwide.

The compound(s) present in the pharmaceutical composition can be present in their unmodified form, salt form, derivative form or a combination thereof. As used herein, the term “derivative” is taken to mean: a) a chemical substance that is related structurally to a first chemical substance and theoretically derivable from it; b) a compound that is formed from a similar first compound or a compound that can be imagined to arise from another first compound, if one atom of the first compound is replaced with another atom or group of atoms; c) a compound derived or obtained from a parent compound and containing essential elements of the parent compound; or d) a chemical compound that may be produced from first compound of similar structure in one or more steps. For example, a derivative may include a deuterated form, oxidized form, dehydrated, unsaturated, polymer conjugated or glycosilated form thereof or may include an ester, amide, lactone, homolog, ether, thioether, cyano, amino, alkylamino, sulfhydryl, heterocyclic, heterocyclic ring-fused, polymerized, pegylated, benzylidenyl, triazolyl, piperazinyl or deuterated form thereof.

As used herein, the term “oleandrin” is taken to mean all known forms of oleandrin unless otherwise specified. Oleandrin can be present in racemic, optically pure or optically enriched form. Nerium oleander plant material can be obtained, for example, from commercial plant suppliers such as Aldridge Nursery, Atascosa, Tex.

The supercritical fluid (SCF) extract can be prepared as detailed in U.S. Pat. Nos. 7,402,325, 8,394,434, 8,187,644, or PCT International Publication No. WP 2007/016176 A2, the entire disclosures of which are hereby incorporated by reference. Extraction can be conducted with supercritical carbon dioxide in the presence or absence of a modifier (organic solvent) such as ethanol.

Other extracts containing cardiac glycoside, especially oleandrin, can be prepared by various different processes. An extract can be prepared according to the process developed by Dr. Huseyin Ziya Ozel (U.S. Pat. No. 5,135,745) describes a procedure for the preparation of a hot water extract. The aqueous extract reportedly contains several polysaccharides with molecular weights varying from 2 KD to 30 KD, oleandrin and oleandrigenin, odoroside and neritaloside. The polysaccharides reportedly include acidic homopolygalacturonans or arabinogalaturonans. U.S. Pat. No. 5,869,060 to Selvaraj et al. discloses hot water extracts of Nerium species and methods of production thereof, e.g. Example 2. The resultant extract can then be lyophilized to produce a powder. U.S. Pat. No. 6,565,897 (U.S. Pregrant Publication No. 20020114852 and PCT International Publication No. WO 2000/016793 to Selvaraj et al.) discloses a hot-water extraction process for the preparation of a substantially sterile extract. Erdemoglu et al. (J. Ethnopharmacol. (2003) November 89(1), 123-129) discloses results for the comparison of aqueous and ethanolic extracts of plants, including Nerium oleander, based upon their anti-nociceptive and anti-inflammatory activities. Organic solvent extracts of Nerium oleander are disclosed by Adome et al. (Aft. Health Sci. (2003) August 3(2), 77-86; ethanolic extract), el-Shazly et al. (J. Egypt Soc. Parasitol. (1996), August 26(2), 461-473; ethanolic extract), Begum et al. (Phytochemistry (1999) February 50(3), 435-438; methanolic extract), Zia et al. (J. Ethnolpharmacol. (1995) November 49(1), 33-39; methanolic extract), and Vlasenko et al. (Farmatsiia. (1972) September-October 21(5), 46-47; alcoholic extract). U.S. Pregrant Patent Application Publication No. 20040247660 to Singh et al. discloses the preparation of a protein stabilized liposomal formulation of oleandrin for use in the treatment of cancer. U.S. Pregrant Patent Application Publication No. 20050026849 to Singh et al. discloses a water soluble formulation of oleandrin containing a cyclodextrin. U.S. Pregrant Patent Application Publication No. 20040082521 to Singh et al. discloses the preparation of protein stabilized nanoparticle formulations of oleandrin from the hot-water extract.

One embodiment of the hot-water extract is available under the tradename ANVIRZEL™ (Nerium Biotechnology, Inc., San Antonio, Tex.; Salud Integral Medical Clinic, Tegucigalpa, Honduras; www.saludintegral.com; www.anvirzel.com) as a liquid dosage form. For sublingual administration, a typical dosing regimen is 1.5 ml per day or three doses of 0.5 ml in one day. For administration by injection, a typical dosing regimen is about 1 to about 2 ml/day, or about 0.1 to about 0.4 ml/m²/day for about 1 week to about 6 months or longer, or about 0.4 to about 0.8 ml/m²/day for about 1 week to about 6 months or longer, or about 0.8 to about 1.2 ml/m²/day for about 1 week to about 6 months or longer. Higher dosing can be used because the maximum tolerated dose of ANVIRZEL™ is much higher. ANVIRZEL™ comprises oleandrin, oleandrigenin, polysaccharides extracted (hot water extraction) from Nerium oleander. Commercially available vials comprise about 150 mg of oleander extract as a freeze-dried powder (prior to reconstitution with water before administration) which comprises about 200 to about 900 microg of oleandrin, about 500 to about 700 microg of oleandrigenin, and polysaccharides extracted from Nerium oleander. Said vials may also include pharmaceutical excipients such as at least one osmotic agent, e.g. mannitol, sodium chloride, at least one buffering agent, e.g. sodium ascorbate with ascorbic acid, at least one preservative, e.g. propylparaben, methylparaben.

The extracts also differ in their polysaccharide and carbohydrate content. The hot water extract contains 407.3 glucose equivalent units of carbohydrate relative to a standard curve prepared with glucose while analysis of the SCF CO₂ extract found carbohydrate levels that were found in very low levels that were below the limit of quantitation. The amount of carbohydrate in the hot water extract of Nerium oleander was, however, at least 100-fold greater than that in the SCF CO₂ extract. The polysaccharide content of the SCF extract can be 0%, <0.5%, <0.1%, <0.05%, or <0.01% wt. In some embodiments, the SCF extract excludes polysaccharide obtained during extraction of the plant mass.

| Nerium oleander | Carbohydrate content |
|---|---|
| preparation | (μg glucose equivalents/mg of plant extract) |
| Hot water extract | 407.3 ± 6.3 |
| SCF CO₂ extract | BLQ (below limit of quantitation) |

The partial compositions of the SCF CO₂ extract and hot water extract were determined by DART TOF-MS (Direct Analysis in Real Time Time of Flight Mass Spectrometry) on a JEOL AccuTOF-DART mass spectrometer (JEOL USA, Peabody, Mass., USA).

The SCF extract of Nerium species is a mixture of pharmacologically active compounds, such as oleandrin and triterpenes. The extract obtained by the SCF process is a substantially water-insoluble, viscous semi-solid (after solvent is removed) at ambient temperature. The SCF extract comprises many different components possessing a variety of different ranges of water solubility. The extract from a supercritical fluid process contains by weight a theoretical range of 0.9% to 2.5% wt of oleandrin or 1.7% to 2.1% wt of oleandrin or 1.7% to 2.0 wt of oleandrin. SCF extracts comprising varying amount of oleandrin have been obtained. In one embodiment, the SCF extract comprises about 2 by wt. of oleandrin. The SCF extract contains a 3-10 fold higher concentration of oleandrin than the hot-water extract. This was confirmed by both HPLC as well as LC/MS/MS (tandem mass spectrometry) analyses.

The SCF extract comprises oleandrin and the triterpenes oleanolic acid, betulinic acid and ursolic acid and optionally other components as described herein. The content of oleandrin and the triterpenes can vary from batch to batch; however, the degree of variation is not excessive. For example, a batch of SCF extract (PB-05204) was analyzed for these four components and found to contain the following approximate amounts of each.

| | | Oleanolic | Ursolic | Betulinic |
|---|---|---|---|---|
| | Oleandrin | acid | acid | acid |
| Content of | 20 | 73 | 69 | 9.4 |
| component | | | | |
| (mg/g of SCF | | | | |
| extract) | | | | |
| Content of | 2 | 7.3 | 6.9 | 0.94 |
| component (% | | | | |
| wt WRT g of | | | | |
| SCT extract) | | | | |
| Content of | 34.7 | 160 | 152 | 20.6 |
| component | | | | |
| (mmole/g of | | | | |
| SCF extract) | | | | |
| Molar ratio of | 1 | 4.6 | 4.4 | 0.6 |
| component | | | | |
| WRT oleandrin | | | | |

WRT denotes “with respect to”.

The content of the individual components may vary by ±25%, ±20%, ±15%, ±10% or ±5% relative to the values indicated. Accordingly, the content of oleandrin in the SCF extract would be in the range of 20 mg±5 mg (which is ±25% of 20 mg) per mg of SCF extract.

An extract of Nerium species was fractionated according to Example 21 (U.S. Pat. No. 9,011,937, issued Apr. 21, 2015 to Addington et al., the entire disclosure of which is hereby incorporated by reference) into five different fractions: O-H, O-2, O-3, O-4 and O-5. The fractions were prepared by loading the unfractionated extract onto an ODS-silica gel column equilibrated with water and subsequently eluting different fractions of the extract by sequentially passing various portions of aqueous mobile phase varying in methanol content (30%, 55%, 80% and 100%) through the column, collecting the respective effluents (fractions) and concentrating the effluents by solvent evaporation under reduced pressure to remove the solvent, thereby providing the fractions O-1 (or O-H), O-2, O-3, O-4 and O-5. The fractions were analyzed and their composition in terms of cardiac glycoside and other components was determined by thin layer chromatography using a sensitive dye indicator that adheres to (and hence is useful for detecting) cardiac glycosides. In addition, the presence or absence of cardiac glycosides in these fractions was analyzed using liquid chromatography/tandem mass spectrometry or DAD-UV detection.

The fractions were also analyzed by HPLC. Based upon a comparison of retention times obtained using corresponding external reference samples, it was determined the (Fr-O-2 and Fr-O-3) fractions contain oleandrin derivatives (cardiac glycosides), oleandrin (Rt=8.3 min) and other unidentified components. The bulk of the oleandrin found in the original unfractionated SCF extract was mainly in the Fr-O-3 fraction. The Fr-0-4 contained no quantifiable amounts of any cardiac glycoside. Accordingly, the composition of the fractions differed according to the content of oleandrin, cardiac glycoside and other unidentified components.

| | Oleandrin | Other Cardiac | Other |
|---|---|---|---|
| Fraction | (Y/N) | Glycoside (Y/N) | components |
| O—H | N | N | Y |
| O-2 | N | Y | Y |
| O-3 | Y | Y | Y |
| O-4 (O-4A) | N | N | Y |
| O-5 | N | N | Y |

A fraction of extract can be sub-fractionated to provide two or more different sub-fractions of a fraction of extract. Sub-fractionation can be carried out by liquid chromatography of the fraction. A suitable stationary phase for liquid chromatography can comprise silica gel or other resins such as ion-exchange media, alumina or nonbonded C18 material and a suitable mobile phase for liquid chromatography can comprise a combination of two or more organic solvents differing in polarity: a less polar organic solvent and a more polar organic solvent. A suitable polar organic solvent can be tetrahydrofuran, dichloromethane, ethyl acetate, acetone, dimethylformamide, acetonitrile, n-butanol, isopropanol, n-propanol, ethanol, methanol, acetic acid and water. A suitable non-polar organic solvent can be ethyl acetate pentane, cyclopentane, hexane, cyclohexane, benzene, toluene, 1,4-dioxane, chloroform or diethyl ether.

Buffering agents for use in buffered solutions include any of those already known in the art of liquid chromatography. Exemplary buffering agents include those containing phosphate, acetate, citrate, formate, phosphate, trifluoroacetic acid, chloroacetate, sulfonate, alkyl amine, TAE, TBE, ammonia, BuffAR, carbonate, HEPES, MES, thiocyanate, CAPS, CHES, guanidine, MOPS, PIPES, TRIS, sulfate, hydroxide, alkali metal halide, tricine, or amino acid ions or combinations thereof. One or more ion-pairing agents and/or one or more organic modifiers can also be included in the mobile phase.

Other types of chromatography that can be used to fractionate the extract include size exclusion chromatography, normal phase chromatography, ion exchange chromatography, hydrophobic interaction chromatography or combinations thereof. It is also possible to use combined forms of different types of chromatography. A stationary phase can include a medium that is a combination of two or more different media used for reverse phase, size exclusion, ion exchange or hydrophobic interaction chromatography, e.g. a combination of reverse phase stationary phase and size exclusion stationary phase, combination of reverse phase stationary phase and ion exchange stationary phase, or other such combinations or two, three or four different stationary phase media. The stationary phase medium can be porous, non-porous, surface porous, diffusive porous or totally porous.

Oleandrin, oleanolic acid, ursolic acid, betulinic acid and derivatives thereof can also be purchased from Sigma-Aldrich (www.sigmaaldrich.com; St. Louis, Mo., USA).

As used herein, the individually named triterpenes can independently be selected upon each occurrence in their native (unmodified, free acid) form, in their salt form, in derivative form, prodrug form, or a combination thereof. Compositions containing and methods employing deuterated forms of the triterpenes are also within the scope of the invention.

Oleanolic acid derivatives, prodrugs and salts are disclosed in US 20150011627 A1 to Gribble et al. which published Jan. 8, 2015, US 20140343108 A1 to Rong et al which published Nov. 20, 2014, US 20140343064 A1 to Xu et al. which published Nov. 20, 2014, US 20140179928 A1 to Anderson et al. which published Jun. 26, 2014, US 20140100227 A1 to Bender et al. which published Apr. 10, 2014, US 20140088188 A1 to Jiang et al. which published Mar. 27, 2014, US 20140088163 A1 to Jiang et al. which published Mar. 27, 2014, US 20140066408 A1 to Jiang et al. which published Mar. 6, 2014, US 20130317007 A1 to Anderson et al. which published Nov. 28, 2013, US 20130303607 A1 to Gribble et al. which published Nov. 14, 2013, US 20120245374 to Anderson et al. which published Sep. 27, 2012, US 20120238767 A1 to Jiang et al. which published Sep. 20, 2012, US 20120237629 A1 to Shode et al. which published Sep. 20, 2012, US 20120214814 A1 to Anderson et al. which published Aug. 23, 2012, US 20120165279 A1 to Lee et al. which published Jun. 28, 2012, US 20110294752 A1 to Arntzen et al. which published Dec. 1, 2011, US 20110091398 A1 to Majeed et al. which published Apr. 21, 2011, US 20100189824 A1 to Arntzen et al. which published Jul. 29, 2010, US 20100048911 A1 to Jiang et al. which published Feb. 25, 2010, and US 20060073222 A1 to Arntzen et al. which published Apr. 6, 2006, the entire disclosures of which are hereby incorporated by reference.

Ursolic acid derivatives, prodrugs and salts are disclosed in US 20150011627 A1 to Gribble et al. which published Jan. 8, 2015, US 20130303607 A1 to Gribble et al. which published Nov. 14, 2013, US 20150218206 A1 to Yoon et al. which published Aug. 6, 2015, U.S. Pat. No. 6,824,811 to Fritsche et al. which issued Nov. 30, 2004, U.S. Pat. No. 7,718,635 to Ochiai et al. which issued May 8, 2010, U.S. Pat. No. 8,729,055 to Lin et al. which issued May 20, 2014, and U.S. Pat. No. 9,120,839 to Yoon et al. which issued Sep. 1, 2015, the entire disclosures of which are hereby incorporated by reference.

Betulinic acid derivatives, prodrugs and salts are disclosed in US 20150011627 A1 to Gribble et al. which published Jan. 8, 2015, US 20130303607 A1 to Gribble et al. which published Nov. 14, 2013, US 20120237629 A1 to Shode et al. which published Sep. 20, 2012, US 20170204133 A1 to Regueiro-Ren et al. which published Jul. 20, 2017, US 20170096446 A1 to Nitz et al. which published Apr. 6, 2017, US 20150337004 A1 to Parthasaradhi Reddy et al. which published Nov. 26, 2015, US 20150119373 A1 to Parthasaradhi Reddy et al. which published Apr. 30, 2015, US 20140296546 A1 to Yan et al. which published Oct. 2, 2014, US 20140243298 A1 to Swidorski et al. which published Aug. 28, 2014, US 20140221328 A1 to Parthasaradhi Reddy et al. which published Aug. 7, 2014, US 20140066416 A1 to Leunis et al. which published Mar. 6, 2014, US 20130065868 A1 to Durst et al. which published Mar. 14, 2013, US 20130029954 A1 to Regueiro-Ren et al. which published Jan. 31, 2013, US 20120302530 A1 to Zhang et al. which published Nov. 29, 2012, US 20120214775 A1 to Power et al. which published Aug. 23, 2012, US 20120101149 A1 to Honda et al. which published Apr. 26, 2012, US 20110224182 to Bullock et al. which published Sep. 15, 2011, US 20110313191 A1 to Hemp et al. which published Dec. 22, 2011, US 20110224159 A1 to Pichette et al. which published Sep. 15, 2011, US 20110218204 to Parthasaradhi Reddy et al. which published Sep. 8, 2011, US 20090203661 A1 to Safe et al. which published Aug. 13, 2009, US 20090131714 A1 to Krasutsky et al. which published May 21, 2009, US 20090076290 to Krasutsky et al. which published Mar. 19, 2009, US 20090068257 A1 to Leunis et al. which published Mar. 12, 2009, US 20080293682 to Mukherjee et al. which published Nov. 27, 2008, US 20070072835 A1 to Pezzuto et al. which published Mar. 29, 2007, US 20060252733 A1 to Jansen et al. which published Nov. 9, 2006, and US 2006025274 A1 to O'Neill et al. which published Nov. 9, 2006, the entire disclosures of which are hereby incorporated by reference.

A pharmaceutical composition can be formulated in any suitable pharmaceutically acceptable dosage form. Parenteral, otic, ophthalmic, nasal, inhalable, buccal, sublingual, enteral, topical, oral, peroral, and injectable dosage forms are particularly useful. Particular dosage forms include a solid or liquid dosage forms. Exemplary suitable dosage forms include tablet, capsule, pill, caplet, troche, sache, solution, suspension, dispersion, vial, bag, bottle, injectable liquid, i.v. (intravenous), i.m. (intramuscular), i.p. (intraperitoneal) intrathecal, intracranial, or intraspanial administrable liquid and other such dosage forms known to the artisan of ordinary skill in the pharmaceutical sciences.

Suitable dosage forms can be prepared by mixing an OCC with pharmaceutically acceptable excipients as described herein or as described in Pi et al. (“Ursolic acid nanocrystals for dissolution rate and bioavailability enhancement: influence of different particle size” in Curr. Drug Deliv. (March 2016), 13(8), 1358-1366), Yang et al. (“Self-microemulsifying drug delivery system for improved oral bioavailability of oleanolic acid: design and evaluation” in Int. J. Nanomed. (2013), 8(1), 2917-2926), Li et al. (Development and evaluation of optimized sucrose ester stabilized oleanolic acid nanosuspensions prepared by wet ball milling with design of experiments” in Biol. Pharm. Bull. (2014), 37(6), 926-937), Zhang et al. (“Enhancement of oral bioavailability of triterpene through lipid nanospheres: preparation, characterization, and absorption evaluation” in J. Pharm. Sci. (June 2014), 103(6), 1711-1719), Godugu et al. (“Approaches to improve the oral bioavailability and effects of novel anticancer drugs berberine and betulinic acid” in PLoS One (March 2014), 9(3):e89919), Zhao et al. (“Preparation and characterization of betulin nanoparticles for oral hypoglycemic drug by antisolvent precipitation” in Drug Deliv. (September 2014), 21(6), 467-479), Yang et al. (“Physicochemical properties and oral bioavailability of ursolic acid nanoparticles using supercritical anti-solvent (SAS) process” in Food Chem. (May 2012), 132(1), 319-325), Cao et al. (“Ethylene glycol-linked amino acid diester prodrugs of oleanolic acid for PEPT1-mediated transport: synthesis, intestinal permeability and pharmacokinetics” in Mol. Pharm. (August 2012), 9(8), 2127-2135), Li et al. (“Formulation, biological and pharmacokinetic studies of sucrose ester-stabilized nanosuspensions of oleanolic acid” in Pharm. Res. (August 2011), 28(8), 2020-2033), Tong et al. (“Spray freeze drying with polyvinylpyrrolidone and sodium caprate for improved dissolution and oral bioavailablity of oleanolic acid, a BCS Class IV compound” in Int. J. Pharm. (February 2011), 404(1-2), 148-158), Xi et al. (Formulation development and bioavailability evaluation of a self-nanoemulsified drug delivery system of oleanolic acid” in AAPS PharmSciTech (2009), 10(1), 172-182), Chen et al. (“Oleanolic acid nanosuspensions: preparation, in-vitro characterization and enhanced hepatoprotective effect” in J. Pharm. Pharmacol. (February 2005), 57(2), 259-264), the entire disclosures of which are hereby incorporated by reference.

Suitable dosage forms can also be made according to U.S. Pat. No. 8,187,644 B2 to Addington, which issued May 29, 2012, U.S. Pat. No. 7,402,325 B2 to Addington, which issued Jul. 22, 2008, U.S. Pat. No. 8,394,434 B2 to Addington et al, which issued Mar. 12, 2013, the entire disclosures of which are hereby incorporated by reference. Suitable dosage forms can also be made as described herein.

An effective amount or therapeutically relevant amount of oleandrin is specifically contemplated. By the term “effective amount”, it is understood that a pharmaceutically effective amount is contemplated. A pharmaceutically effective amount is the amount or quantity of active ingredient which is enough for the required or desired therapeutic response, or in other words, the amount, which is sufficient to elicit an appreciable biological response when, administered to a patient. The appreciable biological response may occur as a result of administration of single or multiple doses of an active substance. A dose may comprise one or more dosage forms. It will be understood that the specific dose level for any patient will depend upon a variety of factors including the indication being treated, severity of the indication, patient health, age, gender, weight, diet, pharmacological response, the specific dosage form employed, and other such factors.

The desired dose for oral administration is up to 5 dosage forms although as few as one and as many as ten dosage forms may be administered as a single dose. Exemplary dosage forms can contain 0.01-100 mg or 0.01-100 microg of the OCC per dosage form, for a total 0.1 to 500 mg (1 to 10 dose levels) per dose. Doses will be administered according to dosing regimens that may be predetermined and/or tailored to achieve specific therapeutic response or clinical benefit in a subject.

The oleandrin can be present in a dosage form in an amount sufficient to provide a subject with an initial dose of oleandrin of about 20 to about 100 microg, about 12 microg to about 300 microg, or about 12 microg to about 120 microg. A dosage form can comprise about 20 of oleandrin to about 100 microg, about 0.01 microg to about 100 mg or about 0.01 microg to about 100 microg oleandrin, oleandrin extract or extract of Nerium oleander containing oleandrin.

The OCC can be included in an oral dosage form. Some embodiments of the dosage form are not enteric coated and release their charge of OCC within a period of 0.5 to 1 hours or less. Some embodiments of the dosage form are enteric coated and release their charge of OCC downstream of the stomach, such as from the jejunum, ileum, small intestine, and/or large intestine (colon). Enterically coated dosage forms will release OCC into the systemic circulation within 1-10 hr after oral administration.

The dosage form can be formulated for rapid release, immediate release, controlled release, sustained release, prolonged release, extended release, burst release, continuous release, slow release, or pulsed release dosage form, or in a dosage form that exhibits two or more of those types of release. The release profile of active agent from the dosage form can be a zero order, pseudo-zero, first order, pseudo-first order or sigmoidal release profile. The plasma concentration profile for the active agent can exhibit one or more maxima in a subject to which the dosage form is administered.

Based on human clinical data it is anticipated that 50% to 75% of an administered dose of oleandrin will be orally bioavailable therefore providing about 10 to about 20 microg, about 20 to about 40 microg, about 30 to about 50 microg, about 40 to about 60 microg, about 50 to about 75 microg, about 75 to about 100 microg of oleandrin per dosage form. Given an average blood volume in adult humans of 5 liters, the anticipated oleandrin plasma concentration will be in the range of about 0.05 to about 2 ng/ml, about 0.005 to about 10 ng/mL, about 0.005 to about 8 ng/mL, about 0.01 to about 7 ng/mL, about 0.02 to about 7 ng/mL, about 0.03 to about 6 ng/mL, about 0.04 to about 5 ng/mL, or about 0.05 to about 2.5 ng/mL. The recommended daily dose of oleandrin, present in the SCF extract, is generally about 0.2 microg to about 4.5 microg/kg bodyweight twice daily. The dose of oleandrin can be about 0.2 to about 1 microg/kg bodyweight/day, about 0.5 to about 1.0 microg/kg bodyweight/day, about 0.75 to about 1.5 microg/kg bodyweight/day, about 1.5 to about 2.52 microg/kg bodyweight/day, about 2.5 to about 3.0 microg/kg bodyweight/day, about 3.0 to 4.0 microg/kg bodyweight/day or about 3.5 to 4.5 microg oleandrin/kg bodyweight/day. The maximum tolerated dose of oleandrin can be about about 3.5 microg/kg bodyweight/day to about 4.0 microg/kg bodyweight/day. The minimum effective dose can be about 0.5 microg/day, about 1 microg/day, about 1.5 microg/day, about 1.8 microg/day, about 2 microg/day, or about 5 microg/day.

The OCC can be administered at low to high dose due to the combination of triterpenes present and the molar ratio at which they are present. A therapeutically effective dose for humans is about 100-1000 mg or about 100-1000 microg of OCC per Kg of bodyweight. Such a dose can be administered up to 10 times in a 24-hour period. Other suitable dosing ranges are specified below.

| | | Oleanolic | Ursolic | Betulinic | |
|---|---|---|---|---|---|
| Compo- | Oleandrin | acid | acid | acid | Suitable |
| sition | (moles) | (moles) | (moles) | (moles) | dose |
| A | 0.5-1.5 | 4-6 | — | — | 0.05 to 0.5 |
| | | | | | mg/kg/day |
| B | 0.5-1.5 | 4-6 | 4-6 | — | 0.05 to 0.35 |
| | | | | | mg/kg/day |
| C | 0.5-1.5 | 4-6 | 4-6 | 0.1-1 | 0.05 to 0.22 |
| (PBI- | | | | | mg/kg/day |
| 05204) | | | | | |
| D | 0.5-1.5 | — | 4-6 | — | 0.05 to 0.4 |
| | | | | | mg/kg/day |
| E | 0.5-1.5 | — | — | 0.1-1 | 0.05 to 0.4 |
| | | | | | mg/kg/day |
| AA | About 1 | — | — | 0.3-0.7 | 0.05 to 0.4 |
| | | | | | mg/kg/day |
| AB | About 1 | About 4.7 | — | — | 0.05 to 0.5 |
| | | | | | mg/kg/day |
| AC | About 1 | About 4.7 | About 4.5 | — | 0.05 to 0.4 |
| | | | | | mg/kg/day |
| AD | About 1 | About 4.7 | About 4.5 | About 0.6 | 0.05 to 0.22 |
| (PBI- | | | | | mg/kg/day |
| 05204) | | | | | |
| AE | About 1 | — | About 4.5 | — | 0.05 to 0.4 |
| | | | | | mg/kg/day |
| AF | About 1 | — | — | About 0.6 | 0.05 to 0.3 |
| | | | | | mg/kg/day |

All values are approximate, meaning “about” the specified value.

It should be noted that a compound herein might possess one or more functions in a composition or formulation of the invention. For example, a compound might serve as both a surfactant and a water miscible solvent or as both a surfactant and a water immiscible solvent.

A liquid composition can comprise one or more pharmaceutically acceptable liquid carriers. The liquid carrier can be an aqueous, non-aqueous, polar, non-polar, and/or organic carrier. Liquid carriers include, by way of example and without limitation, a water miscible solvent, water immiscible solvent, water, buffer and mixtures thereof.

As used herein, the terms “water soluble solvent” or “water miscible solvent”, which terms are used interchangeably, refer to an organic liquid which does not form a biphasic mixture with water or is sufficiently soluble in water to provide an aqueous solvent mixture containing at least five percent of solvent without separation of liquid phases. The solvent is suitable for administration to humans or animals. Exemplary water soluble solvents include, by way of example and without limitation, PEG (poly(ethylene glycol)), PEG 400 (poly(ethylene glycol having an approximate molecular weight of about 400), ethanol, acetone, alkanol, alcohol, ether, propylene glycol, glycerin, triacetin, poly(propylene glycol), PVP (poly(vinyl pyrrolidone)), dimethylsulfoxide, N,N-dimethylformamide, formamide, N,N-dimethylacetamide, pyridine, propanol, N-methylacetamide, butanol, soluphor (2-pyrrolidone), pharmasolve (N-methyl-2-pyrrolidone).

As used herein, the terms “water insoluble solvent” or “water immiscible solvent”, which terms are used interchangeably, refer to an organic liquid which forms a biphasic mixture with water or provides a phase separation when the concentration of solvent in water exceeds five percent. The solvent is suitable for administration to humans or animals. Exemplary water insoluble solvents include, by way of example and without limitation, medium/long chain triglycerides, oil, castor oil, corn oil, vitamin E, vitamin E derivative, oleic acid, fatty acid, olive oil, softisan 645 (Diglyceryl Caprylate/Caprate/Stearate/Hydroxy stearate adipate), miglyol, captex (Captex 350: Glyceryl Tricaprylate/Caprate/Laurate triglyceride; Captex 355: Glyceryl Tricaprylate/Caprate triglyceride; Captex 355 EP/NF: Glyceryl Tricaprylate/Caprate medium chain triglyceride).

Suitable solvents are listed in the “International Conference on Harmonisation of Technical Requirements for Registration of Pharmaceuticals for Human Use (ICH) guidance for industry Q3C Impurities: Residual Solvents” (1997), which makes recommendations as to what amounts of residual solvents are considered safe in pharmaceuticals. Exemplary solvents are listed as class 2 or class 3 solvents. Class 3 solvents include, for example, acetic acid, acetone, anisole, 1-butanol, 2-butanol, butyl acetate, tert-butlymethyl ether, cumene, ethanol, ethyl ether, ethyl acetate, ethyl formate, formic acid, heptane, isobutyl acetate, isopropyl acetate, methyl acetate, methyl-1-butanol, methylethyl ketone, methylisobutyl ketone, 2-methyl-1-propanol, pentane, 1-pentanol, 1-propanol, 2-propanol, or propyl acetate.

Other materials that can be used as water immiscible solvents in the invention include: Captex 100: Propylene Glycol Dicaprate; Captex 200: Propylene Glycol Dicaprylate/Dicaprate; Captex 200 P: Propylene Glycol Dicaprylate/Dicaprate; Propylene Glycol Dicaprylocaprate; Captex 300: Glyceryl Tricaprylate/Caprate; Captex 300 EP/NF: Glyceryl Tricaprylate/Caprate Medium Chain Triglycerides; Captex 350: Glyceryl Tricaprylate/Caprate/Laurate; Captex 355: Glyceryl Tricaprylate/Caprate; Captex 355 EP/NF: Glyceryl Tricaprylate/Caprate Medium Chain Triglycerides; Captex 500: Triacetin; Captex 500 P: Triacetin (Pharmaceutical Grade); Captex 800: Propylene Glycol Di (2-Ethythexanoate); Captex 810 D: Glyceryl Tricaprylate/Caprate/Linoleate; Captex 1000: Glyceryl Tricaprate; Captex CA: Medium Chain Triglycerides; Captex MCT-170: Medium Chain Triglycerides; Capmul GMO: Glyceryl Monooleate; Capmul GMO-50 EP/NF: Glyceryl Monooleate; Capmul MCM: Medium Chain Mono- & Diglycerides; Capmul MCM C8: Glyceryl Monocaprylate; Capmul MCM C10: Glyceryl Monocaprate; Capmul PG-8: Propylene Glycol Monocaprylate; Capmul PG-12: Propylene Glycol Monolaurate; Caprol 10G10O: Decaglycerol Decaoleate; Caprol 3GO: Triglycerol Monooleate; Caprol ET: Polyglycerol Ester of Mixed Fatty Acids; Caprol MPGO: Hexaglycerol Dioleate; Caprol PGE 860: Decaglycerol Mono-, Dioleate.

As used herein, a “surfactant” refers to a compound that comprises polar or charged hydrophilic moieties as well as non-polar hydrophobic (lipophilic) moieties; i.e., a surfactant is amphiphilic. The term surfactant may refer to one or a mixture of compounds. A surfactant can be a solubilizing agent, an emulsifying agent or a dispersing agent. A surfactant can be hydrophilic or hydrophobic.

The hydrophilic surfactant can be any hydrophilic surfactant suitable for use in pharmaceutical compositions. Such surfactants can be anionic, cationic, zwitterionic or non-ionic, although non-ionic hydrophilic surfactants are presently preferred. As discussed above, these non-ionic hydrophilic surfactants will generally have HLB values greater than about 10. Mixtures of hydrophilic surfactants are also within the scope of the invention.

Similarly, the hydrophobic surfactant can be any hydrophobic surfactant suitable for use in pharmaceutical compositions. In general, suitable hydrophobic surfactants will have an HLB value less than about 10. Mixtures of hydrophobic surfactants are also within the scope of the invention.

Examples of additional suitable solubilizer include: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol, available commercially from BASF under the trade name Tetraglycol) or methoxy PEG (Union Carbide); amides, such as 2-pyrrolidone, 2-piperidone, caprolactam, N-alkylpyrrolidone, N-hydroxyalkylpyrrolidone, N-alkylpiperidone, N-alkylcaprolactam, dimethylacetamide, and polyvinypyrrolidone; esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, caprolactone and isomers thereof, valerolactone and isomers thereof, butyrolactone and isomers thereof; and other solubilizers known in the art, such as dimethyl acetamide, dimethyl isosorbide (Arlasolve DMI (ICI)), N-methyl pyrrolidones (Pharmasolve (ISP)), monooctanoin, diethylene glycol nonoethyl ether (available from Gattefosse under the trade name Transcutol), and water. Mixtures of solubilizers are also within the scope of the invention.

Except as indicated, compounds mentioned herein are readily available from standard commercial sources.

Although not necessary, the composition or formulation may further comprise one or more chelating agents, one or more preservatives, one or more antioxidants, one or more adsorbents, one or more acidifying agents, one or more alkalizing agents, one or more antifoaming agents, one or more buffering agents, one or more colorants, one or more electrolytes, one or more salts, one or more stabilizers, one or more tonicity modifiers, one or more diluents, or a combination thereof.

The composition of the invention can also include oils such as fixed oils, peanut oil, sesame oil, cottonseed oil, corn oil and olive oil; fatty acids such as oleic acid, stearic acid and isostearic acid; and fatty acid esters such as ethyl oleate, isopropyl myristate, fatty acid glycerides and acetylated fatty acid glycerides. The composition can also include alcohol such as ethanol, isopropanol, hexadecyl alcohol, glycerol and propylene glycol; glycerol ketals such as 2,2-dimethyl-1,3-dioxolane-4-methanol; ethers such as poly(ethylene glycol) 450; petroleum hydrocarbons such as mineral oil and petrolatum; water; a pharmaceutically suitable surfactant, suspending agent or emulsifying agent; or mixtures thereof.

It should be understood that compounds used in the art of pharmaceutical formulation generally serve a variety of functions or purposes. Thus, if a compound named herein is mentioned only once or is used to define more than one term herein, its purpose or function should not be construed as being limited solely to that named purpose(s) or function(s).

One or more of the components of the formulation can be present in its free base, free acid or pharmaceutically or analytically acceptable salt form. As used herein, “pharmaceutically or analytically acceptable salt” refers to a compound that has been modified by reacting it with an acid as needed to form an ionically bound pair. Examples of acceptable salts include conventional non-toxic salts formed, for example, from non-toxic inorganic or organic acids. Suitable non-toxic salts include those derived from inorganic acids such as hydrochloric, hydrobromic, sulfuric, sulfonic, sulfamic, phosphoric, nitric and others known to those of ordinary skill in the art. The salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and others known to those of ordinary skill in the art. On the other hand, where the pharmacologically active ingredient possesses an acid functional group, a pharmaceutically acceptable base is added to form the pharmaceutically acceptable salt. Lists of other suitable salts are found in Remington's Pharmaceutical Sciences, 17^{th}. ed., Mack Publishing Company, Easton, Pa., 1985, p. 1418, the relevant disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with tissues of human beings and animals and without excessive toxicity, irritation, allergic response, or any other problem or complication, commensurate with a reasonable benefit/risk ratio.

A dosage form can be made by any conventional means known in the pharmaceutical industry. A liquid dosage form can be prepared by providing at least one liquid carrier and OCC in a container. One or more other excipients can be included in the liquid dosage form. A solid dosage form can be prepared by providing at least one solid carrier and OCC. One or more other excipients can be included in the solid dosage form.

A dosage form can be packaged using conventional packaging equipment and materials. It can be included in a pack, bottle, via, bag, syringe, envelope, packet, blister pack, box, ampoule, or other such container.

The composition of the invention can be included in any dosage form. Particular dosage forms include a solid or liquid dosage forms. Exemplary suitable dosage forms include tablet, capsule, pill, caplet, troche, sache, and other such dosage forms known to the artisan of ordinary skill in the pharmaceutical sciences.

In view of the above description and the examples below, one of ordinary skill in the art will be able to practice the invention as claimed without undue experimentation. The foregoing will be better understood with reference to the following examples that detail certain procedures for the preparation of embodiments of the present invention. All references made to these examples are for the purposes of illustration. The following examples should not be considered exhaustive, but merely illustrative of only a few of the many embodiments contemplated by the present invention.

### Example 1

### Supercritical Fluid Extraction of Powdered Oleander Leaves

### Method A. With Carbon Dioxide.

Powdered oleander leaves were prepared by harvesting, washing, and drying oleander leaf material, then passing the oleander leaf material through a comminuting and dehydrating apparatus such as those described in U.S. Pat. Nos. 5,236,132, 5,598,979, 6,517,015, and 6,715,705. The weight of the starting material used was 3.94 kg.

The starting material was combined with pure CO₂ at a pressure of 300 bar (30 MPa, 4351 psi) and a temperature of 50° C. (122° F.) in an extractor device. A total of 197 kg of CO₂ was used, to give a solvent to raw material ratio of 50:1. The mixture of CO₂ and raw material was then passed through a separator device, which changed the pressure and temperature of the mixture and separated the extract from the carbon dioxide.

The extract (65 g) was obtained as a brownish, sticky, viscous material having a nice fragrance. The color was likely caused by chlorophyll and other residual chromophoric compounds. For an exact yield determination, the tubes and separator were rinsed out with acetone and the acetone was evaporated to give an addition 9 g of extract. The total extract amount was 74 g. Based on the weight of the starting material, the yield of the extract was 1.88%. The content of oleandrin in the extract was calculated using high pressure liquid chromatography and mass spectrometry to be 560.1 mg, or a yield of 0.76%.

### Method B. With Mixture of Carbon Dioxide and Ethanol

Powdered oleander leaves were prepared by harvesting, washing, and drying oleander leaf material, then passing the oleander leaf material through a comminuting and dehydrating apparatus such as those described in U.S. Pat. Nos. 5,236,132, 5,598,979, 6,517,015, and 6,715,705. The weight of the starting material used was 3.85 kg.

The starting material was combined with pure CO₂ and 5% ethanol as a modifier at a pressure of 280 bar (28 MPa, 4061 psi) and a temperature of 50° C. (122° F.) in an extractor device. A total of 160 kg of CO₂ and 8 kg ethanol was used, to give a solvent to raw material ratio of 43.6 to 1. The mixture of CO₂, ethanol, and raw material was then passed through a separator device, which changed the pressure and temperature of the mixture and separated the extract from the carbon dioxide.

The extract (207 g) was obtained after the removal of ethanol as a dark green, sticky, viscous mass obviously containing some chlorophyll. Based on the weight of the starting material, the yield of the extract was 5.38%. The content of oleandrin in the extract was calculated using high pressure liquid chromatography and mass spectrometry to be 1.89 g, or a yield of 0.91%.

### Example 2

### Hot-Water Extraction of Powdered Oleander Leaves

### Comparative Example

Hot water extraction is typically used to extract oleandrin and other active components from oleander leaves. Examples of hot water extraction processes can be found in U.S. Pat. Nos. 5,135,745 and 5,869,060.

A hot water extraction was carried out using 5 g of powdered oleander leaves. Ten volumes of boiling water (by weight of the oleander starting material) were added to the powdered oleander leaves and the mixture was stirred constantly for 6 hours. The mixture was then filtered and the leaf residue was collected and extracted again under the same conditions. The filtrates were combined and lyophilized. The appearance of the extract was brown. The dried extract material weighed about 1.44 g. 34.21 mg of the extract material was dissolved in water and subjected to oleandrin content analysis using high pressure liquid chromatography and mass spectrometry. The amount of oleandrin was determined to be 3.68 mg. The oleandrin yield, based on the amount of extract, was calculated to be 0.26%.

### Example 3

### Preparation of Pharmacotherapy Compositions

### Method A. Cremophor-Based Drug Delivery System

The following ingredients were provided in the amounts indicated.

| Reagent | | Percent of Formulation |
|---|---|---|
| Name | Function | (% w/w) |
| OCC | Active agent | 3.7 |
| Vitamin E | Antioxidant | 0.1 |
| Labrasol | Surfactant | 9.2 |
| Ethanol | Co-solvent | 9.6 |
| Cremophor EL | Surfactant | 62.6 |
| Cremophor RH40 | Surfactant | 14.7 |

The excipients were dispensed into ajar and shook in a New Brunswick Scientific C24KC Refrigerated Incubator shaker for 24 hours at 60° C. to ensure homogeneity. The samples were then pulled and visually inspected for solubilization. Both the excipients and OCC were totally dissolved for all formulations after 24 hours.

### Method B. GMO Cremophor-Based Drug Delivery System

The following ingredients were provided in the amounts indicated.

| Reagent | | Percent of Formulation |
|---|---|---|
| Name | Function | (% w/w) |
| OCC | Active agent | 4.7 |
| Vitamin E | Antioxidant | 0.1 |
| Labrasol | Surfactant | 8.5 |
| Ethanol | Co-solvent | 7.6 |
| Cremophor EL | Surfactant | 56.1 |
| Glycerol Monooleate | Surfactant | 23.2 |

The procedure of Method A was followed.

### Method C. Labrasol-Based Drug Delivery System

The following ingredients were provided in the amounts indicated.

| | | Percent of |
|---|---|---|
| Reagent | | Formulation |
| Name | Function | (% w/w) |
| OCC | Active agent | 3.7 |
| Vitamin E | Antioxidant | 0.1 |
| Labrasol | Surfactant | 86.6 |
| Ethanol | Co-solvent | 9.6 |

The procedure of Method A was followed.

### Method D. Vitamin E-TPGS Based Micelle Forming System

The following ingredients were provided in the amounts indicated.

| | | Weight % |
|---|---|---|
| Component | Function | (w/w) |
| Vitamin E | Antioxidant | 1.0 |
| Vitamin E TPGS | Surfactant | 95.2 |
| OCC | Active agent | 3.8 |

The procedure of Method A was followed.

### Method E. Multi-Component Drug Delivery System

The following ingredients were provided in the amounts indicated.

| | Weight | Weight % |
|---|---|---|
| Component | (g) | (w/w) |
| Vitamin E | 10.0 | 1.0 |
| Cremophor ELP | 580.4 | 55.9 |
| Labrasol | 89.0 | 8.6 |
| Glycerol Monooleate | 241.0 | 23.2 |
| Ethanol | 80.0 | 7.7 |
| OCC | 38.5 | 3.7 |
| Total | 1038.9 | 100 |

The procedure of Method A was followed.

### Method F. Multi-Component Drug Delivery System

The following ingredients were provided in the amounts indicated an included in a capsule.

| | | Weight % |
|---|---|---|
| Component | Tradename | (w/w) |
| OCC | FLAVEX Naturextrakte | 0.6 |
| Vitamin E | | 1.3 |
| Caprylocaproyl | Labrasol | 11.1 |
| polyoxyglycerides | Gattefosse 3074TPD | |
| Lauroyl | Gelucire 44/14 | 14.6 |
| polyoxyglycerides | Gattefosse 3061TPD | |
| Polyoxyl 35 Castor | Kolliphor | 72.4 |
| oil | BASF Corp. 50251534 | |
| Total | | 100 |

The procedure of Method A was followed.

### Example 4

### Preparation of Enteric Coated Capsules

### Step I: Preparation of Liquid-Filled Capsule

Hard gelatin capsules (50 counts, 00 size) were filled with a liquid composition of Example 3. These capsules were manually filled with 800 mg of the formulation and then sealed by hand with a 50% ethanol/50% water solution. The capsules were then banded by hand with 22% gelatin solution containing the following ingredients in the amounts indicated.

| Ingredient | Wt. (g) |
|---|---|
| Gelatin | 140.0 |
| Polysorbate 80 | 6.0 |
| Water | 454.0 |
| Total | 650.0 |

The gelatin solution mixed thoroughly and allowed to swell for 1-2 hours. After the swelling period, the solution was covered tightly and placed in a 55° C. oven and allowed to liquefy. Once the entire gelatin solution was liquid, the banding was performed

Using a pointed round 3/0 artist brush, the gelatin solution was painted onto the capsules. Banding kit provided by Shionogi was used. After the banding, the capsules were kept at ambient conditions for 12 hours to allow the band to cure.

### Step II: Coating of Liquid-Filled Capsule

A coating dispersion was prepared from the ingredients listed in the table below.

| Ingredient | Wt. % | Solids % | Solids (g) | g/Batch |
|---|---|---|---|---|
| Eudragit L30D55 | 40.4 | 60.5 | 76.5 | 254.9 |
| TEC | 1.8 | 9.0 | 11.4 | 11.4 |
| AlTalc 500 V | 6.1 | 30.5 | 38.5 | 38.5 |
| Water | 51.7 | na | na | 326.2 |
| Total | 100.0 | 100.0 | 126.4 | 631.0 |

If banded capsules according to Step I were used, the dispersion was applied to the capsules to a 20.0 mg/cm² coating level. The following conditions were used to coat the capsules.

| Parameters | Set-up | |
|---|---|---|
| Coating Equipment | Vector LDCS-3 | |
| Batch Size | 500 | g |
| Inlet Air Temp. | 40° | C. |
| Exhaust Air Temp. | 27-30° | C. |
| Inlet Air Volume | 20-25 | CFM |
| Pan Speed | 20 | rpm |
| Pump Speed | 9 rpm (3.5 to 4.0 g/min) | |
| Nozzle Pressure | 15 | psi |
| Nozzle diameter | 1.0 | mm |
| Distance from tablet bed* | 2-3 | in |

*Spray nozzle was set such that both the nozzle and spray path were under the flow path of inlet air.

### Example 5

### Treatment of GBM with PBI-05204: Mouse Orthotopic Injection Model

### Radiotherapy and Pharmacotherapy

Mice (10) were orthotopically injected with GBM tumor cells (IC1128 or IC3752). After a two-week of tumor development, fractionated X-ray therapy (XRT for five days) and pharmacotherapy (chronic administration of PBI-05204; 25 mg/Kg; i.p. for 28 days) was initiated (FIG. 1A). The mice were divided into four groups: Group 1: control-received no radiotherapy or pharmacotherapy; Group 2: received PBI-05204; Group 3: received XRT; and Group 4: received XRT and PBI-05204.

Survival was quantified for the four groups and the results for mice injected with IC1128 GBM are detailed in FIG. 1B.

### Example 6

### Treatment of GBM with PBI-05204: Mouse Orthotopic Injection Model

### Radiotherapy, Chemotherapy, and Pharmacotherapy

The brain tissue of mice was orthotopically injected with GBM tumor cells (U87). After tumor development (followed via florescent imaging without the need to sacrifice the mice), animals received no treatment (controls), PBI-05204 (40 mg/kg/day orally 5 days/week for 5 weeks), temozolomide (TMZ, 32 mg/kg (4 consecutive days, days 9-12), radiotherapy (XRT, single dose of 4 Gy on day 10) or combinations of these treatments. The combination protocol of FIG. 2A was followed.

The mice were divided into 8 groups: Group 1: control—received only vehicle and no XRT, TMZ, or PBI-05204; Group 2: received TMZ and no XRT or PBI-05204; Group 3: received PBI-05204 in vehicle and no XRT or TMZ; Group 4: received XRT and no TMZ or PBI-05204; Group 5: received XRT and PBI-05204 and no TMZ; Group 6: received PBI-05204 and TMZ; Group 7: received XRT and TMZ; and Group 8: received XRT, TMZ, and PBI-05204.

Overall survival was quantified for the eight groups and the results are detailed in FIGS. 2B-2D.

### Example 7

### Preparation of a Tablet Comprising Pharmacotherapy Composition

An initial tabletting mixture of 3% Syloid 244FP and 97% microcrystalline cellulose (MCC) was mixed. Then, an existing batch of composition prepared according to Example 3 was incorporated into the Syloid/MCC mixture via wet granulation. This mixture is labeled “Initial Tabletting Mixture) in the table below. Additional MCC was added extra-granularly to increase compressibility. This addition to the Initial Tabletting Mixture was labeled as “Extra-granular Addition.” The resultant mixture from the extra-granular addition was the same composition as the “Final Tabletting Mixture.”

| | Weight | Weight % |
|---|---|---|
| Component | (g) | (w/w) |
| Initial Tabletting Mixture | | |
| Microcrystalline cellulose | 48.5 | 74.2 |
| Colloidal Silicon Dioxide/ | 1.5 | 2.3 |
| Syloid 244FP | | |
| Formulation from Ex. 3 | 15.351 | 23.5 |
| Total | 65.351 | 100.0 |

Extragranular Addition

| | Weight | Weight % |
|---|---|---|
| Component | (g) | (w/w) |
| Initial Tabulating Mixture | 2.5 | 50.0 |
| Microcrystalline cellulose | 2.5 | 50.0 |
| Total | 5 | 100.0 |

Final Tabletting Mixture:

Abbreviated

| | Weight | Weight % |
|---|---|---|
| Component | (g) | (w/w) |
| Microcrystalline cellulose | 4.36 | 87.11 |
| Colloidal Silicon Dioxide/ | 0.06 | 1.15 |
| Syloid 244FP | | |
| Formulation from Ex. 3 | 0.59 | 11.75 |
| Total | 5.00 | 100 |

Final Tabletting Mixture:

Detailed

| | Weight | Weight % |
|---|---|---|
| Component | (g) | (w/w) |
| Microcrystalline cellulose | 4.36 | 87.11 |
| Colloidal Silicon Dioxide/ | 0.06 | 1.15 |
| Syloid 244FP | | |
| Vitamin E | 0.01 | 0.11 |
| Cremophor ELP | 0.33 | 6.56 |
| Labrasol | 0.05 | 1.01 |
| Glycerol Monooleate | 0.14 | 2.72 |
| Ethanol | 0.05 | 0.90 |
| SCF extract | 0.02 | 0.44 |
| Total | 5.00 | 100.00 |

Syloid 244FP is a colloidal silicon dioxide manufactured by Grace Davison. Colloidal silicon dioxide is commonly used to provide several functions, such as an adsorbant, glidant, and tablet disintegrant. Syloid 244FP was chosen for its ability to adsorb 3 times its weight in oil and for its 5.5 micron particle size.

### Example 8

### HPLC Analysis of Solutions Containing Oleandrin

Samples (oleandrin standard, SCF extract and hot-water extract) were analyzed on HPLC (Waters) using the following conditions: Symmetry C18 column (5.0 μm, 150×4.6 mm I.D.; Waters); Mobile phase of MeOH:water=54:46 (v/v) and flow rate at 1.0 ml/min. Detection wavelength was set at 217 nm. The samples were prepared by dissolving the compound or extract in a fixed amount of HPLC solvent to achieve an approximate target concentration of oleandrin. The retention time of oleandrin can be determined by using an internal standard. The concentration of oleandrin can be determined/calibrated by developing a signal response curve using the internal standard.

### Example 9

### Preparation of Pharmacotherapy Composition

A pharmaceutical composition of the invention can be prepared any of the following methods. Mixing can be done under wet or dry conditions. The pharmaceutical composition can be compacted, dried or both during preparation. The pharmaceutical composition can be portioned into dosage forms.

### Method A.

At least one pharmaceutical excipient is mixed with at least oleandrin.

### Method B.

At least one pharmaceutical excipient is mixed with at least oleandrin-containing extract as disclosed herein.

### Method C.

At least one pharmaceutical excipient is mixed with at least oleandrin, at least one other active ingredient extracted with oleandrin from oleandrin-containing plant material, and at least one chemotherapeutic agent.

### Method D.

At least one pharmaceutical excipient is mixed with at least oleandrin and at least two triterpenes as disclosed herein.

### Method E.

At least one pharmaceutical excipient is mixed with at least oleandrin and at least three triterpenes as disclosed herein.

### Example 10

### Preparation of Triterpene Mixtures

The following compositions were made by mixing the specified triterpenes in the approximate molar ratios indicated.

| | Triterpene (Approximate | | |
|---|---|---|---|
| | Relative Molar Content) | | |
| | Oleanolic | Ursolic | Betulinic |
| Composition | acid (O) | acid (U) | acid (B) |
| I (A-C) | 3 | 2.2 | 1 |
| II (A-C) | 7.8 | 7.4 | 1 |
| III (A-C) | 10 | 1 | 1 |
| IV (A-C) | 1 | 10 | 1 |
| V (A-C) | 1 | 1 | 10 |
| VI (A-C) | 1 | 1 | 0 |
| VII (A-C) | 1 | 1 | 1 |
| VIII (A-C) | 10 | 1 | 0 |
| IX (A-C) | 1 | 10 | 0 |

For each composition, three different respective solutions were made, whereby the total concentration of triterpenes in each solution was approximately 9 μM, 18 μM, or 36 μM.

| Composition | | | |
|---|---|---|---|
| (total | Triterpene (Approximate | | |
| triterpene | Content of Each, μM) | | |
| content, | Oleanolic | Ursolic | Betulinic |
| μM) | acid (O) | acid (U) | acid (B) |
| I-A (36) | 17.4 | 12.8 | 5.8 |
| I-B (18) | 8.7 | 6.4 | 2.9 |
| I-C (9) | 4.4 | 3.2 | 1.5 |
| II-A (36) | 17.3 | 16.4 | 2.2 |
| II-B (18) | 8.7 | 8.2 | 1.1 |
| II-C (9) | 4.3 | 4.1 | 0.6 |
| III-A (36) | 30 | 3 | 3 |
| III-B (18) | 15 | 1.5 | 1.5 |
| III-C (9) | 7.5 | 0.75 | 0.75 |
| IV-A (36) | 3 | 30 | 3 |
| IV-B (18) | 1.5 | 15 | 1.5 |
| IV-C (9) | 0.75 | 7.5 | 0.75 |
| V-A (36) | 3 | 3 | 30 |
| V-B (18) | 1.5 | 1.5 | 15 |
| V-C (9) | 0.75 | 0.75 | 7.5 |
| VI-A (36) | 18 | 18 | 0 |
| VI-B (18) | 9 | 9 | 0 |
| VI-C (9) | 4.5 | 4.5 | 0 |
| VII-A (36) | 12 | 12 | 12 |
| VII-B (18) | 6 | 6 | 6 |
| VII-C (9) | 3 | 3 | 3 |
| VIII-A (36) | 32.7 | 3.3 | 0 |
| VIII-B (18) | 16.35 | 1.65 | 0 |
| VIII-C (9) | 8.2 | 0.8 | 0 |
| IX-A (36) | 3.3 | 32.7 | 0 |
| IX-B (18) | 1.65 | 16.35 | 0 |
| IX-C (9) | 0.8 | 8.2 | 0 |

### Example 11

### Preparation of Pharmacotherapy Compositions

Anticancer compositions can be prepared by mixing the individual triterpene components thereof to form a mixture. The triterpene mixtures prepared above that were formulated into pharmacotherapy compositions.

Anticancer Composition with Oleanolic Acid and Ursolic Acid

Known amounts of oleanolic acid and ursolic acid were mixed according to a predetermined molar ratio of the components as defined herein. The components were mixed in solid form or were mixed in solvent(s), e.g. methanol, ethanol, chloroform, acetone, propanol, dimethyl sulfoxide (DMSO), dimethylformamide (DMF), dimethylacetamide (DMAC), N-methylpyrrolidone (NMP), water or mixtures thereof. The resultant mixture contained the components in the relative molar ratios as described herein.

For a pharmaceutically acceptable OCC, at least one pharmaceutically acceptable excipient was mixed in with the pharmacologically active agents. An anticancer composition is formulated for administration to a mammal.

Anticancer Composition with Oleanolic Acid and Betulinic Acid

Known amounts of oleanolic acid and betulinic acid were mixed according to a predetermined molar ratio of the components as defined herein. The components were mixed in solid form or were mixed in solvent(s), e.g. methanol, ethanol, chloroform, acetone, propanol, dimethyl sulfoxide (DMSO), dimethylformamide (DMF), dimethylacetamide (DMAC), N-methylpyrrolidone (NMP), water or mixtures thereof. The resultant mixture contained the components in the relative molar ratios as described herein.

For a pharmaceutically acceptable OCC, at least one pharmaceutically acceptable excipient was mixed in with the pharmacologically active agents. An anticancer composition is formulated for administration to a mammal.

Anticancer Composition with Oleanolic Acid, Ursolic Acid, and Betulinic Acid

Known amounts of oleanolic acid, ursolic acid and betulinic acid were mixed according to a predetermined molar ratio of the components as defined herein. The components were mixed in solid form or were mixed in solvent(s), e.g. methanol, ethanol, chloroform, acetone, propanol, dimethyl sulfoxide (DMSO), dimethylformamide (DMF), dimethylacetamide (DMAC), N-methylpyrrolidone (NMP), water or mixtures thereof. The resultant mixture contained the components in the relative molar ratios as described herein.

For a pharmaceutically acceptable OCC, at least one pharmaceutically acceptable excipient was mixed in with the pharmacologically active agents. An anticancer composition is formulated for administration to a mammal.

Anticancer Composition with Oleadrin, Oleanolic Acid, Ursolic Acid, and Betulinic Acid

Known amounts of oleandrin, oleanolic acid, ursolic acid and betulinic acid were mixed according to a predetermined molar ratio of the components as defined herein. The components were mixed in solid form or were mixed in solvent(s), e.g. methanol, ethanol, chloroform, acetone, propanol, dimethyl sulfoxide (DMSO), dimethylformamide (DMF), dimethylacetamide (DMAC), N-methylpyrrolidone (NMP), water or mixtures thereof. The resultant mixture contained the components in the relative molar ratios as described herein.

For a pharmaceutically acceptable OCC, at least one pharmaceutically acceptable excipient was mixed in with the pharmacologically active agents. An anticancer composition is formulated for administration to a mammal.

### Example 12

### Cell Lines and Cell Cultures

Materials for tissue culture were purchased either from Euroclone Italia (Euroclone S.p.A, Milan, Italy) or from ATCC (Manassas, Va.). Three human glioma cell lines (U251, U87MG and T98G) were cultured at 37° C. in 5% CO₂ in Dulbecco's modified Eagle medium (DMEM) containing 10% (v/v) fetal bovine serum, 4 mM glutamine, 100 IU/ml penicillin, 100 microg/ml streptomycin and 1% nonessential amino acids (Thermo Fisher Scientific Inc., Carlsbad, Calif., USA). The risk of working with misidentified and/or contaminated cell lines was minimized by using GBM cells at very low passages and periodic short tandem repeat (STR) DNA profiling. Luciferase tagged U87MG cells were generated and provided by Jari E. Heikkila (Abo Akademi University, Turku, Finland). A GBM patient-derived stem cell line (BT48EF) was provided by J. Gregory Cairncross and Samuel Weiss (University of Calgary, Canada). Isolated neurospheres of U87 cells were assayed for ‘stemness’ properties in terms of clonogenic capacity and positivity for stem cell markers.

### Reagents and Enzymatic Activities

Antibodies against total Akt (Sc-377457), p-AktSer473 (sc-135651), p-AktThr308 (sc-135650), Phospho-S6 Ribosomal Protein (pSer235/236-56) or pSer2448-4E-BP1, anti-human CD31 (PECAM-1, clone M-20, sc-1506), betaIII tubulin (clone 3H3091, sc-69966), and SOX2 (clone A-5, sc-365964) were purchased from Santa Cruz Biotechnology (Santa Cruz, Calif., USA). Antibodies against Ki67 (Clone MIB-1, M7240) were purchased from Dako (Agilent Technologies Italia S.P.A., Cernusco sul Naviglio, Milan, Italy). Antibodies against CD44 (Cell signaling, #357259) and SOX2 for IHC staining (cell signaling, #14926s) were purchased from Cell signaling. The murine CD31 (clone MEC 7.46, ab7388) and CXCR4 antibodies were purchased from Abcam (Cambridge, UK or Cambridge, Mass.). Cell-based enzyme-linked immunosorbent assays (ELISAs) for total and phosphorylated isoforms of Akt (Ser473 and Thr308), Ser65 p-4E-BP 1 and Ser235/236 p-S6 were used for detecting and quantifying target proteins in cultured cells following the “In-Cell ELISA protocol” (Abcam).

### Example 13

### Growth Assays and Viability: Neurosphere Proliferation

### Evaluation of PBI-05204

Twenty-four-well plates were seeded with 2×10⁴ GBM cells/mL. After cells were attached and grown in DMEM cell culture medium with 5% fetal calf serum (FCS) for 24 h, they were treated with different concentrations of PBI-05204. A Nikon Diaphot inverted phase-contrast photomicroscope (Nikon Corp., Tokyo, Japan) was used to monitor cell morphology before cell trypsinization and counting. Cell counts were made with a NucleoCounter NC-100 (Chemotec, Gydevang, Denmark). IC₅₀ values, the concentration of drug required for a 50% reduction in growth/viability, were calculated using the Dojindo Cell Counting Kit-8 (Dojindo EU GmbH, Munich, Germany). For neurosphere proliferation two different modalities of study were used: (i) a direct count and sizing of neurospheres at 1 week of culture from pre-formed spheres, and (ii) an evaluation of the clonal capacity of cancer stem cells cultured as single cells after 14-30 days. For the analysis of sphere growth, pre-formed neurospheres were treated with different doses of PBI-05204 for 72 hr. After treatment, spheres were photographed and counted using phase contrast microscopy. Spheres were recorded as either large colonies (>50 cells) or small colonies (<50 cells). Single cells were also manually counted per microscopic field at 100× magnification. For the clonogenic assay, glioma tumor-initiating cells (GICs) were seeded in 96-well plates as a single cell suspension at a density of 2 cells/ml (equivalent to 1 cell every 3 wells). Cells were maintained for 14-30 days in their culturing media and then the wells were visually scanned by light microscopy to identify and count the clones (spheres) produced.

Evaluation of Non-Oleadrin Components of Extracted from Nerium Species

The method above is repeated, except that compositions comprising one or more components extractable (extracted) from Nerium species are used instead of PBI-05204. Said one or more components are listed in this specification (vide supra). This same procedure was used to evaluate the triterpenes individually and as combinations.

### Example 14

### Cell Cycle Assay

### Evaluation of PBI-05204

For cell cycle analysis, cells (2.5×10⁶) grown in 100-mm dishes were treated with PBI-05204 (0.5-2.5 μg/ml) for 24 hr. Cells were subjected to trypsinization and centrifugation, and the pellets were suspended and washed in 1× phosphate-buffered saline solution (PBS) and were fixed overnight in 70% ethanol at 4° C. The cells were then washed with 1×PBS and were suspended in PBTB staining solution containing PBS, 0.5% bovine serum albumin (BSA), 0.005% Tween-20, propidium iodide (10 μg/ml) and DNase-free RNase (1 μg/ml). Cells were incubated in the dark for 30 min at 37° C. before fluorescence-activated cell-sorting (FACS) analysis using a BD FACS Caliber flow cytometer (BD Biosciences, San Jose, Calif.). The percentage of cells in each phase of the cell cycle was estimated from the DNA histogram content. Apoptosis was evaluated by APOSTRAND™ ELISA apoptosis detection kit (3V Chimica S.r.l. Rome, Italy) and by caspase-specific chromogenic substrates at 450 nm in an ELISA plate reader such as Ac-DEVD-pNA (caspase-3), Ac-IETD-pNA (caspase 8) and Ac-LEHD-pNA (caspase 9) purchased from Kaneka Eurogentec SA (Seraing, Belgium).

Evaluation of Non-Oleadrin Components of Extracted from Nerium Species

The method above is repeated, except that compositions comprising one or more components extractable (extracted) from Nerium species are used instead of PBI-05204. Said one or more components are listed in this specification (vide supra).

### Example 15

### Immunofluorescence Assays

Glioma stem like cells were used for immuno-fluorescence analyses. Spheres were seeded at a density of 10,000 cells/cm² on glass coverslips pretreated with 30 μg/ml Poly-L lysine to promote adherence. The slides were then washed twice with phosphate-buffered saline (PBS) and fixed with 4% paraformaldehyde for 20 min at room temperature (RT). To stain cytoplasmic markers, slides were permeabilized with 0.3% Triton-X-100 for 5 minutes at RT. Spheres were then incubated overnight at 4° C. with the following primary antibodies accordingly to their data sheets: betaIII tubulin, SOX2, CXCR4 and CD44. After washing with PBS, cells were incubated for 30 minutes at RT with AlexaFluor 488 anti-rabbit IgG, AlexaFluor 595 anti-goat IgG or AlexaFluor 633 anti-mouse IgG secondary antibody (1:2000; Molecular Probes, Invitrogen, Carlsbad, Calif., USA). Controls were performed by omitting the primary antibody. Cell nuclei were stained with DAPI (0.5 g/ml). Coverslips were mounted with Vectashield Mounting Medium and examined with a Leica TCS SP5 confocal microscope (Leica Microsystems Inc., Mannheim, Germany).

### Example 16

### Immunoblotting

Cell extracts were obtained from treated or untreated cultures, washed with cold PBS and subjected to lysis buffer containing proteinase and phosphatase inhibitor cocktails. Proteins were subjected to 7% or 15% sodium dodecyl sulphate-polyacrylamide gel electrophoresis (SDS-PAGE), transferred to nitrocellulose and probed with appropriate antibodies as per recommendations of the suppliers. Reactive bands were visualized with a chemiluminescent detection kit (Perbio Science, Tattenhall, UK) in a Bio-Rad gel Doc system (Bio-Rad Laboratories S.r.l., Milan, Italy) or visualized using Pierce ECL Plus substrate (Thermo Fisher Scientific, Waltham, Mass., USA). Normalization of specific bands was performed using an anti-tubulin or anti-β-actin antibody.

### Example 17

### In Vivo Xenograft Mouse Model

### Evaluation of PBI-05204

Female CD1-nu/nu mice, at 6 weeks of age, were purchased from Charles River (Milan, Italy) under guidelines established by the University of L'Aquila, Medical School and Science and Technology School Board Regulations (complying with the Italian government regulation n. 116 Jan. 27, 1992 for the use of laboratory animals). All mice received subcutaneous flank injections (2 each) of 1×10⁶ U251, U87MG or T98G cells. Tumor growth was assessed twice a week by measuring tumor diameters with a Vernier caliper. Xenografts were considered to be equivalent to an ovoid having three diameters: the formula used was ‘TW (mg)=tumor volume (mm³)=4/3πR1×R2×R3 in which R1/R2/R3 are rays of an ellipsoid. Shorter diameter is the thickness/height of tumor, larger diameters are the length and width of tumor. About 10 days after the tumor injection, 20 mice with tumor volumes of 0.8˜1.3 cm³ were retained and randomly divided into 4 groups (5 mice per group with two tumors each). Treatment groups consisted of: (1) Control (vehicle); (2) PBI-05204 (10 mg/kg, 5 Day/week, PO); (3) PBI-05204 (20 mg/kg, 5 Day/week, PO); and (4) PBI-05204 (40 mg/kg, 5 Day/week, PO). At the end of experiments (35 days after initial treatment) animals were sacrificed by carbon dioxide inhalation and tumors were subsequently removed surgically. Half of the tumor was directly frozen in liquid nitrogen for protein analysis and the other half fixed in paraformaldehyde overnight for immunohistochemical analyses.

Evaluation of Non-Oleadrin Components of Extracted from Nerium Species

The method above is repeated, except that compositions comprising one or more components extractable (extracted) from Nerium species are used instead of PBI-05204. Said one or more components are listed in this specification (vide supra).

### Example 18

### In Vivo Orthotopic Intra-Brain Mouse Model

### Evaluation of PBI-05204

Female CD1 nu/nu mice were inoculated intra-cerebrally with luciferase transfected established U87MG cells. Just before treatment initiation (5 days after injection), animals were randomized to treatment groups of 10 mice each. In vivo bioluminescence images were obtained using the UVITEC Cambridge Mini HD6 (UVItec Limited, Cambridge, United Kingdom). Animals were anesthetized and luciferin (150 mg/kg) was injected intra-peritoneally (IP) 15 min prior to imaging. The mice were photographed while placed on their front and the bioluminescence intensity (BLI) was measured in the region of interest. Treatments were started 5 days after cell injection when no luciferase activity was intracranially detectable. Mice received PBI-05204 orally over 35 days with a 45-day non-drug follow up period. Mice were euthanized when they displayed neurological signs (e.g., altered gait, tremors/seizures, lethargy) or weight loss of 20% or greater of pre-surgical weight.

DSF (disease-free survival) was defined as the time during which no bioluminescence evidence of tumour was recorded. Repeated bioluminescence assays were performed in order to monitor tumour progression. Treatments were completed after 35 days when a period without drug administration was started. Relative bioluminescence signal detection was associated with low, intermediate and large intra-brain tumors at necroscopy. Overall survival (OS), defined as the time (days) prior to which an animal did not show the distress signs cited above or was equal to the time of euthanasia. Brains were collected, fixed with 4% paraformaldehyde and paraffin embedded.

Evaluation of Non-Oleadrin Components of Extracted from Nerium Species

The method above is repeated, except that compositions comprising one or more components extractable (extracted) from Nerium species are used instead of PBI-05204. Said one or more components are listed in this specification (vide supra).

### Example 19

### Immunohistochemical Analysis

Indirect immunoperoxidase staining was performed on 4 m paraffin-embedded tissue sections. Tumor microvessels were counted at ×400 in five arbitrarily selected fields and the data were presented as number of CD31+ mouse microvessels/×100 microscopic field for each group. Ki67 labeling index was determined by counting 500 cells at 100× and determining the percentage of cells staining positively for Ki67. Apoptosis was measured as the percentage of tunnel positive cells measured on five random fields (400×) by using TACS Blue Label kit (R&D Systems, Inc., Minneapolis, Minn., USA).

### Example 20

### Statistical Analysis

Continuous variables were summarized as mean and standard deviation (SD) or as median with 95% confidence intervals (CI). For continuous variables not normally distributed, statistical comparisons between control and treated groups were established by carrying out the Kruskal-Wallis test and Dwass-Steel-Chritchlow-Fligner method. For continuous variables normally distributed, statistical comparisons between control and treated groups were established by carrying out an analysis of variance (ANOVA) test or by Student t test for unpaired data (for 2 comparisons). When the ANOVA test revealed a statistical difference, pair-wise comparisons were made by Tukey's honestly significant difference (HSD) test. Overall survival was analysed by Kaplan-Meier curves and Gehan's generalized Wilcoxon test. When more than 2 survival curves were compared the logrank test for trend was used. P values <0.05 were considered statistically significant. MedCalc (MedCalc Software, Ostend, Belgium) was used as a complete statistical program.

### Example 21

### Compositions Comprising One or More Components Extractable from Nerium Species

A supercritical extract (5 g) of oleander leaves (obtained as described herein by extracting a plant mass with a mixture of supercritical CO₂ with EtOH added as a cosolvent/modifier, Batch #270111) was suspended in water (150 mL) and partitioned three times with hexane (150 ml each time). The water layer was subjected to ODS C-18 (octadecyl-functionalized silica gel, 20-22% labeled, 200-400 mesh) open column (400 mm (L)×38 mm (ID)) fractionation by charging the water layer directly to a bed of the ODS resin equilibrated with water. The column was treated successively with mixtures of water and methanol (1000 ml of 30% methanol in water, 1000 ml of 55% methanol in water, 1000 ml of 80% methanol in water, 1000 ml of 100% methanol) and with a mixture of acetone:methanol (2 volumes:1 volume; 1000 ml). The effluent (1000 ML) from each mixture was collected. The solvent was removed from each fraction by evaporation to yield five fractions, namely Fr-O-1, Fr-O-2, Fr-O-3, Fr-O-4, and Fr-O-5. The fractions were then analyzed by HPLC chromatography.

### Example 22

### HPLC Analysis of Fractions of SCF Extract

The purpose of this assay was to identify extract fractions (from above) containing cardiac glycoside. A sample from each fraction obtained according to Example 13 was analyzed as follows. The fraction 1-3 mg) was dissolved in 1-5 ml of aqueous methanol (80% methanol in water). The diluted sample (10-25 μl) was analyzed with an Agilent Zorbax SB-C18 column using 80% methanol in water as the mobile phase, a flow rate of 0.7 mL/min and DAD-UV effluent monitoring at the following wavelengths: 203, 210, 217, 230, 254, 280, 310 and 300 nm.

### Example 23

### Identification of Compounds in a Fraction of Nerium oleander SCF Extract

The water and methanol present in the Fr-O-4 fraction were removed by evaporation under reduced pressure. The residue from the Fr-O-4 fraction of Example 13 was subjected to silica gel chromatography (below) to provide sub-fractions that were then analyzed by thin layer chromatography (TLC). Fractions having similar TLC profiles were combined and the solvents thereof removed by evaporation under reduced pressure. The remaining residues were analyzed by HNMR.

### Thin Layer Chromatography

TLC was performed on conventional analytical grade TLC plates using a mixture of hexane:ethyl acetate (7:3 v:v). The compounds were visualized with H₂SO₄, whereby steroids exhibit a blue color and triterpenes exhibit a purple color.

Prior to further fractionation by flash chromatography, TLC analysis of the Fr-O-4 fraction indicated the presence of one major spot and more than five small spots. The color reaction indicated that the major spot contained a mixture of steroid and triterpene and most of the small spots contained steroids.

### Silica Gel Flash Chromatography

Silica gel (Biotage; (10-15 g) was loaded into a column and equilibrated with a mixture of ethyl acetate (3%) and hexane (97%). The residue from the Fr-O-4 fraction was taken up in mixture 0.2-0.5 ml of ethyl acetate (3%) and hexane (97%) and charged onto the column. Flash chromatography was conducted using a solvent gradient of ethyl acetate (3%-30%) in hexane (97%-70%, respectively) followed by 100% methanol. Sub-fractions collected from the column were analyzed by TLC (above) and those fractions having similar TLC visualization profiles were combined and concentrated to remove solvent.

### HNMR Spectroscopy

A sample of each of the concentrated sub-fractions obtained from flash chromatography was analyzed by HNMR using conventional methods so as to determine the structural class for the major components.

### Example 24

### Identification of Compounds in Nerium oleander SCF Extract Obtained According to Example 1 (Method B) in Unfractionated Form

The SCF extract was analyzed by MS-DART TOF analysis as follows. A JEOL AccuTOF-DART mass spectrometer (Jeol U.S.A., Peobody, Mass., U.S.A.) was used.

A JEOL AccuTOF-DART mass spectrometer (Jeol USA, Peabody, Mass., USA) was used. Analyses were conducted in a positive ion mode (DART+) giving masses corresponding to the M+H+ ions generated by the DART-MS. A range of settings on the instrument was used to determine optimal conditions for N. oleander analyses. The general settings for DART+ included: needle voltage 3500 V; orifice 1-2-20 V; ring lens 2-5 V; orifice 2-2-5 V; and peaks voltage 1000 V. Calibrations were performed internally with each sample using a 10% solution of PEG 600 which provides mass markers throughout the required mass range of 100-1000 mass units. Other analyses were undertaken in the DART-mode and these consisted of: needle voltage 3500 V; heating element 250° C.; electrode 1—150 V; electrode 2—250 V; He gas flow rate 3.79 LPM. Mass spectrometer settings: MCP 2600 V; orifice 1—15 V; ring lens—5 V, orifice 2—5 V; and peaks voltage 1000 V. Calibrations were performed internally with each sample using a perfluorinated carboxylic acid solution that provides markers throughout the required mass range of 100-1000 mass units. The N. oleander samples were introduced neat into the DART helium plasma using the closed end of a borosilicate glass melting point tube. The capillary tube was held in the He plasma for approximately 3-5 s per analysis. Molecular formulas were confirmed by elemental composition and isotope matching programs provided with the JEOL AccuTOF DART-MS instrument. A searchable database of N. oleander constituents, developed by HerbalScience (Naples, Fla., USA) was used.

The SCF extract was found to contain at least the following components present in the indicated relative abundances (%).

| | Relative |
|---|---|
| Component | Abundance (%) |
| Oleandrin | 2.99 |
| Oleandrigenin | 3.31 |
| Ursolic acid/betulinic acid | 15.29 |
| Odoroside | 0.80 |
| Oleanolic acid | 0.60 |
| Urs-12-ene-3β,28-diol/betulin | 5.44 |
| 3β,3β-hydroxy-12-olean-en-28-oic acid | 14.26 |
| 28-norurs-12-en-3β-ol | 4.94 |
| Urs-12-en-3β-ol | 4.76 |

### Example 25

### Growth Assays and Viability; High Throughput Screening Assay

### Evaluation of Triterpenes Individually and as Varying Combinations

A total of 500 GBM9 cells or 1,000 GS28 cells per well suspended in 50 ul of media are seeded into Greiner Black 384-well plates (Cat #781091) using Multidrop Combi liquid dispenser (ThermoFisher).

The cells are allowed to recover and form a monolayer overnight at 37 C in a humidified chamber with 5% CO₂.

After recovery, 50 nl of each drug are transferred into well using an Echo 550 acoustic dispensing platform (Labcyte).

In the combination screen, two or three drugs are tested with a fixed volume of DMSO (0.2% v/v) and two biological replicates. Each assay plate contains a fixed concentration of drug in addition to a negative control (0.1% DMSO), a positive control (10 uM Doxorubicin), and an 8-point dose response curve of the positive controls. Each well image is taken using the ImageXpress Micro Conforcal High Content Imaging System (MolecularDevices) everyday four times.

After 72 hr incubation in the presence of drug, plates are aspirated for left 25 ul and then, 25 ul of Cell Titer Glo-3D (Promega) are added and incubated for 20 minutes. Assay plates are read on the Synergy Neo2 Hybrid Multi-Mode Microplate Reader (BioTek).

### Example 26

### Spheroids Formation Assay or Stem Cells Renewal Assay in Glioblastoma Stem Cells (GSCs)

Evaluation of Combination of Triterpenoic Acids with or without Oleandrin

500 glioblastoma stem cells (GSCs) were plated in each well in 24-well plate in triplicates in each individual cell line under different treatment spheroids assays. Tested agents at the concentration indicated in the figures were added to the medium 72 hrs after the cells were plated and was kept for additional 96 hrs to monitor the progression of sphere formation. The sphere formation status was acquired by live cell imaging using EVOS FL phase contrast microscope (Thermo Fischer Scientific). GSC Spheres were counted in each well (5 wells per each condition) and the average number of spheroids were calculated as the average number of spheres/well. The size of the spheroids was also measured using the ImageJ software.

### Example 27

### Growth and Proliferation Assay

### Evaluation of Combination of Triterpenoic Acids in GBM Cells

96-well plates were seeded with 6×104 GBM cells/mL. After cells were attached and grown in DMEM cell culture medium with 10% fetal bovine serum (FBS) for 24 h, they were treated with three different concentrations of combination of ursolic acid, betulinic acid and oleanoic acid (4:1:16). The starting concentration (defined as 1×) of ursolic acid, betulinic acid and oleanoic acid 1.58, 0.4 and 6.25 μM, respectively. After an additional 72 hr, inhibition of cellular proliferation was assessed by MTT assay. Absorbance was read at a wavelength of 570 nm and a reference wavelength of 650 nm using a VMax Microplate Reader (Molecular Devices, Inc., Sunnyvale, Calif.).

### Example 28

### Growth and Proliferation Assay

### Method for Assessing Cell Proliferation in GBM Cells in the Presence of OUB

Human glioblastoma U87 and U251 cells were obtained from the American Type Culture Collection (ATCC, Manassas, Va.) and were maintained in a humidified atmosphere containing 5% carbon dioxide at 37° C. U87 and U251 cells were routinely cultured in Dulbecco modified Eagle medium supplemented with 10% heat-inactivated fetal bovine serum (HyClone Laboratories, Inc., Logan, Utah), penicillin (10 IU/ml, Invitrogen/Thermo Fisher Scientific, Carlsbad, Calif.), and streptomycin (10 μg/ml, Invitrogen/Thermo Fisher Scientific).

U87 and U251 cells (8×10³) were plated in 96-well plates. After incubating for 24 hr, cells were treated with various concentrations of oleanoic acid (2.85-45.67 μg/ml), betulinic acid (1.42-45.67 μg/ml), ursolic acid (1.42-45.67 μg/ml), and combination of oleanoic acid, betulinic acid and ursolic acid (10:1:1, 3.13-100 μg/ml). After an additional 72 hr of incubation, inhibition of cellular proliferation was assessed by MTT assay. Absorbance was read at a wavelength of 570 nm and a reference wavelength of 650 nm using a VMax Microplate Reader (Molecular Devices, Inc., Sunnyvale, Calif.).

As used herein, the term “about” or “approximately” are taken to mean ±10%, ±5%, ±2.5% or ±1% of a specified valued. For example, “about 20%” is taken to mean 20±2%, 20±1%, 20±0.5%, or 20±0.2%. As used herein, the term “substantially” is taken to mean “to a large degree” or “at least a majority of” or “more than 50% of”.

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except as by the appended claims. All of the embodiments disclosed and claimed herein can be made and executed without undue experimentation in light of the present disclosure.

1) (canceled)

2) A combination protocol method for the treatment of GM, in particular GBM, comprising at least the following steps, which may be executed in any order during a treatment period:
treating said subject with radiotherapy; and/or treating said subject with chemotherapy; and
treating said subject with pharmacotherapy employing a composition comprising a) oleandrin; b) oleanolic acid (OA), ursolic acid (UA), and betulinic acid (BA), and oleandrin; or c) oleanolic acid (OA), ursolic acid (UA), and betulinic acid (BA), and excluding oleandrin;
wherein oleandrin is present in native form or prodrug form; and
optionally resecting the GM.

3) (canceled)

4) (canceled)

5) (canceled)

6) (canceled)

7) (canceled)

8) The method of claim 2, wherein a) radiotherapy is X-ray radiotherapy; and b) chemotherapy is temozolomide (TMZ) chemotherapy.

9) The method of claim 8, wherein said step of resecting is conducted and said steps of treating are conducted in ay order during a treatment period.

10) The method of claim 2, wherein a) radiotherapy is conducted repeatedly during a treatment period; b) chemotherapy is conducted repeatedly during a treatment period; c) pharmacotherapy is conducted repeatedly during a treatment period; d) radiotherapy and chemotherapy are conducted in an overlapping manner during a treatment period; e) radiotherapy and pharmacotherapy are conducted in an overlapping manner during a treatment period; f) chemotherapy and pharmacotherapy are conducted in an overlapping manner during a treatment period; g) radiotherapy, chemotherapy and pharmacotherapy are conducted in an overlapping manner during a treatment period; h) radiotherapy and pharmacotherapy are conducted in a sequential manner during a treatment period; i) chemotherapy and pharmacotherapy are conducted in a sequential manner during a treatment period; j) radiotherapy, chemotherapy and pharmacotherapy are conducted in a sequential manner during a treatment period; k) resection of the tumor is conducted before any one of radiotherapy, chemotherapy and pharmacotherapy; l) resection of the tumor is conducted after any one of radiotherapy, chemotherapy and pharmacotherapy; m) resection of the tumor is conducted between radiotherapy and chemotherapy; n) resection of the tumor is conducted between radiotherapy and pharmacotherapy; o) resection of the tumor is conducted between chemotherapy and pharmacotherapy, or p) any combination of the above.

11) The method of claim 9, wherein said resecting leaves a resection site in said subject; and the following steps are conducted in an overlapping manner during a treatment period:
administering TMZ to said subject;
irradiating tissue defining and surrounding said resection site with X-ray radiation; and
administering said composition to said subject.

12) (canceled)

13) The method of claim 2, wherein a) said radiotherapy comprises fractionated X-ray radiotherapy for a period of at least about 5 days; and said pharmacotherapy comprises chronically administering an oleandrin-containing composition (OCC) to said subject on a daily basis for a period of at least about 28 days; b) said pharmacotherapy comprises chronically administering OCC on a daily basis to said subject five days per week for at least five weeks, said radiotherapy comprises treating said subject to at least a dose of X-ray radiation, and said chemotherapy comprises treating said subject to at least three doses of temozolomide (TMZ); c) said pharmacotherapy comprises chronically administering to said subject OCC, said radiotherapy comprises irradiating the GM of said subject with X-ray radiation at least once, and said chemotherapy comprises administering at least a dose of TMZ to said subject without resecting the GM from said subject; d) said pharmacotherapy comprises chronically administering OCC to said subject, said radiotherapy comprises irradiating the GM of said subject with X-ray radiation at least once, and said chemotherapy comprises administering at least a dose of TMZ to said subject, and resecting the GM from said subject any time prior to or after said administering OCC; e) said pharmacotherapy comprises chronically administering OCC to said subject, said radiotherapy comprises irradiating the GM of said subject with X-ray radiation wherein the total dose of radiation is fractionated over two or more days, and said chemotherapy comprises administering plural doses of TMZ to said subject; f) said pharmacotherapy comprises administering plural doses of OCC to said subject, said radio therapy comprises irradiating the GM of said subject with plural doses of X-ray radiation, and said chemotherapy comprises administering plural doses of TMZ to said subject, said method optionally further comprising resecting the GM; g) said composition comprises oleandrin and said resecting is not conducted; or h) said composition comprises oleandrin and said pharmacotherapy is conducted prior to or after resecting the GM from said subject.

14) (canceled)

15) (canceled)

16) (canceled)

17) (canceled)

18) (canceled)

19) (canceled)

20) (canceled)

21) (canceled)

22) The method of claim 2, wherein a) the total dose of TMZ is evenly divided over two or more days; and/or b) the total dose of said composition is evenly divided over two or more days.

23) The method of claim 2, wherein said composition is selected from the group consisting of a) a pharmaceutical composition comprising oleandrin; b) a pharmaceutical composition comprising extract of plant material, said extract comprising oleandrin; c) a pharmaceutical composition comprising extract of plant material, said extract comprising oleandrin and one or more other active ingredients extractable from said plant material; d) a pharmaceutical composition comprising extract of Nerium sp. plant material, said extract comprising oleandrin; e) a pharmaceutical composition comprising extract of Nerium sp. plant material, said extract comprising oleandrin and one or more other active ingredients extractable from said plant material; and f) any combination of any two or more of these listed items.

24) The method of claim 23, wherein said extract is a supercritical fluid extract, a water extract, an organic solvent extract, or a combination of any of these listed extracts.

25) The method of claim 2, wherein chemotherapy comprises at least administering to a subject one or more chemotherapeutic agents known or found to be therapeutically effective against GM.

26) The method of claim 25, wherein said one or more other chemotherapeutic agents are selected from the group consisting of nitrosoureas, DNA alkylating agent(s), temozolomide (TMZ), carmustine (BCNU), lomustine (CCNU), nimustine (ACNU), fotemusine, cediranib, erlotinib, galunisertib, irinotecan, procarbazine, vincristine, bevacizumab, hydroxyurea, and cytarabine.

27) The method of claim 2, wherein said composition comprises oleandrin and at least one or more active ingredients selected from the group consisting of cardiac glycoside, glycone, aglycone, steroid, triterpene, polysaccharide, saccharide, neritaloside, odoroside, oleanolic acid, ursolic acid, betulinic acid, oleandrigenin, oleaside A, betulin (urs-12-ene-3β,28-diol), 28-norurs-12-en-3β-ol, urs-12-en-3β-ol, 3β,3β-hydroxy-12-oleanen-28-oic acid, 3β,20α-dihydroxyurs-21-en-28-oic acid, 3β,27-dihydroxy-12-ursen-28-oic acid, 3β,13β-dihydroxyurs-11-en-28-oic acid, 3β,12α-dihydroxyoleanan-28,13β-olide, 3β,27-dihydroxy-12-oleanan-28-oic acid, homopolygalacturonan, arabinogalaturonan, chlorogenic acid, caffeic acid, L-quinic acid, 4-coumaroyl-CoA, 3-O-caffeoylquinic acid, 5-O-caffeoylquinic acid, cardenolide B-1, cardenolide B-2, oleagenin, neridiginoside, nerizoside, odoroside-H, 3-beta-O-(D-diginosyl)-5-beta, 14 beta-dihydroxy-card-20(22)-enolide; pectic polysaccharide composed of galacturonic acid, rhamnose, arabinose, xylose, and galactose; polysaccharide with MW in the range of 17000-120000 D, or MW about 35000 D, about 3000 D, about 5500 D, or about 12000 D; cardenolide monoglycoside, cardenolide N-1, cardenolide N-2, cardenolide N-3, cardenolide N-4, pregnane, 4,6-diene-3,12,20-trione, 20R-hydroxypregna-4,6-diene-3,12-dione, 16beta,17beta-epoxy-12beta-hydroxypregna-4,6-diene-3,20-dione, 12beta-hydroxypregna-4,6,16-triene-3,20-dione (neridienone A), 20S,21-dihydroxypregna-4,6-diene-3,12-dione (neridienone B), neriucoumaric acid, isoneriucoumaric acid, oleanderoic acid, oleanderen, 8alpha-methoxylabdan-18-oic acid, 12-ursene, kaneroside, neriumoside, 3β-O-(D-diginosyl)-2α-hydroxy-8,14β-epoxy-5β-carda-16:17, 20:22-dienolide, 3β-O-(D-diginosyl)-2α,14β-dihydroxy-5β-carda-16:17,20:22-dienolide, 3β,27-dihydroxy-urs-18-en-13,28-olide, 3β,22α,28-trihydroxy-25-nor-lup-1(10),20(29)-dien-2-one, cis-karenin (3β-hydroxy-28-Z-p-coumaroyloxy-urs-12-en-27-oic acid), trans-karenin (3-β-hydroxy-28-E-p-coumaroyloxy-urs-12-en-27-oic acid), 3beta-hydroxy-5alpha-carda-14(15),20(22)-dienolide (beta-anhydroepidigitoxigenin), 3 beta-O-(D-digitalosyl)-21-hydroxy-5beta-carda-8,14,16,20(22)-tetraenolide (neriumogenin-A-3beta-D-digitaloside), proceragenin, neridienone A, 3beta,27-dihydroxy-12-ursen-28-oic acid, 3beta,13beta-dihydroxyurs-11-en-28-oic acid, 3beta-hydroxyurs-12-en-28-aldehyde, 28-orurs-12-en-3beta-ol, urs-12-en-3beta-ol, urs-12-ene-3beta,28-diol, 3beta,27-dihydroxy-12-oleanen-28-oic acid, (20S,24R)-epoxydammarane-3beta,25-diol, 20beta,28-epoxy-28alpha-methoxytaraxasteran-3beta-ol, 20beta,28-epoxytaraxaster-21-en-3beta-ol, 28-nor-urs-12-ene-3beta,17 beta-diol, 3beta-hydroxyurs-12-en-28-aldehyde, alpha-neriursate, beta-neriursate, 3alpha-acetophenoxy-urs-12-en-28-oic acid, 3beta-acetophenoxy-urs-12-en-28-oic acid, oleanderolic acid, kanerodione, 3β-p-hydroxyphenoxy-11α-methoxy-12α-hydroxy-20-ursen-28-oic acid, 28-hydroxy-20(29)-lupen-3,7-dione, kanerocin, 3alpha-hydroxy-urs-18,20-dien-28-oic acid, D-sarmentose, D-diginose, neridiginoside, nerizoside, isoricinoleic acid, gentiobiosylnerigoside, gentiobiosylbeaumontoside, gentiobiosyloleandrin, folinerin, 12β-hydroxy-5β-carda-8,14,16,20(22)-tetraenolide, 8β-hydroxy-digitoxigenin, Δ16-8β-hydroxy-digitoxigenin, Δ16-neriagenin, uvaol, ursolic aldehyde, 27(p-coumaroyloxy)ursolic acid, oleanderol, and combination thereof.

28) The method of claim 2, wherein GM is selected from the group consisting of astrocytoma (which include low-grade astrocytomas (Grade I pilocytic astrocytoma, and Grade II difuse astrocytoma), anaplastic astrocytoma (Grade III), glioblastoma (Grade IV, GBM, also known as glioblastoma multiforme)), ependymoma, and oligodendroglioma.

29) (canceled)

30) (canceled)

31) The method of claim 2, wherein the dosing protocol for said radiotherapy or for said irradiating said subject with X-ray is selected from the group consisting of a) fractionated XRT, whereby a total dose of radiation is divided and administered over a predetermined period of days, weeks, or months; b) the subject receiving a dose of X-ray radiation daily for at least one day or for a first period of 1-7, 1-6, 1-5, 1-4, 1-3, or 1-2 days; c) the subject not being exposed to X-ray radiation for at least one day or for a second period of 1-2, 1-3, 1-4, 1-5, 1-6, or 1-7 days; and optionally d) steps (items) b) and c) are repeated at least once, at least twice, at least three time, as least four times, at least five times, at least six times; e) a total dose of about 60 Gy is divided and administered over about 30 daily fractions; f) a total dose of about 60 Gy is administered in about 2 Gy/day fractions over a period of about 30 days; g) a total dose of about 46 Gy is administered in about 2 Gy/day fractions over a period of about 23 days; h) a total dose of about 14 Gy is administered in about 2 Gy/day fractions over a period of about 7 days; i) a total dose of about 35 Gy is divided and administered over about 10 daily fractions; j) a total dose of about 30 Gy is divided and administered over about 5 daily fractions; k) a total dose of about 40 Gy is divided and administered over about 15 fractions over about 3 weeks; 1) a total dose of about 50 Gy is divided into fractions of about 1.5-2 Gy and administered over about 25 to 35 day; m) a total dose of about 36 Gy is administered in about 2 Gy/day fractions; n) a total dose of about 60 Gy is administered in about 2 Gy/day fractions over a period of about 6 weeks.

32) The method of claim 2, wherein the radiotherapy includes focal XRT of GM or GBM tumor bed or resection site with: a) an about 2 to about 3 cm margin; and/or b) an about 2-cm CTV (computed tomographic venography) margin and an about 3-mm to about 5-mm PTV (planning target volume) margin.

33) The method of claim 8, wherein the dosing protocol for said chemotherapy or for said administering TMZ to said subject is selected from the group consisting of a) 75 mg TMZ/m²/day for 6 weeks; b) six maintenance cycles of 150-200 mg of TMZ/m²/day for the first five days of a 28-day cycle; c) any one or more of the dosing schedules detailed in US NDA 021029 for the TEMODAR® dosage form; d) 75-100 mg TMVZ/m²/day for days 1-21 of a 28-day cycle for 6 to 12 cycles; e) 200 mg TMVZ/m²/day for about 5 consecutive days per 28-day treatment cycle; or f) a combination of any two or more of the listed dosing schedules.

34) The method according to claim 33, wherein the daily dose of TMZ is selected from any of the following:

| Total | | | |
|---|---|---|---|
| BSA | 75 mg/m² | 150 mg/m² | 200 mg/m² |
| (m²) | (mg daily) | (mg daily) | (mg daily) |
| 1.0 | 75 | 150 | 200 |
| 1.1 | 82.5 | 165 | 220 |
| 1.2 | 90 | 180 | 240 |
| 1.3 | 97.5 | 195 | 260 |
| 1.4 | 105 | 210 | 280 |
| 1.5 | 112.5 | 225 | 300 |
| 1.6 | 120 | 240 | 320 |
| 1.7 | 127.5 | 255 | 340 |
| 1.8 | 135 | 270 | 360 |
| 1.9 | 142.5 | 285 | 380 |
| 2.0 | 150 | 300 | 400 |
| 2.1 | 157.5 | 315 | 420 |
| 2.2 | 165 | 330 | 440 |
| 2.3 | 172.5 | 345 | 460 |
| 2.4 | 180 | 360 | 480 |
| 2.5 | 187.5 | 375 | 500 . |

35) The method of claim 2, wherein said composition is an oleandrin-containing composition (OCC) and the dosing protocol for said OCC pharmacotherapy or for said administering OCC to said subject is selected from the group consisting of a) chronic daily doses over a period of days; b) chronic daily administration over a period of at least one week, at least two weeks, at least three weeks, at least four weeks, at least five weeks, at least six weeks or more; c) administered maintenance doses of OCC after completion of radiotherapy and chemotherapy; d) daily doses of OCC for a period of at least four weeks or at least one month; e) daily doses of OCC for at least a first period of days, weeks, or months; f) daily doses of OCC for at least one day or for at least a period of 1-7, 1-6, 1-5, 1-4, 1-3, or 1-2 days; g) no daily doses of OCC for at least one day or for at least a period of 1-7, 1-6, 1-5, 1-4, 1-3, or 1-2 days; or h) a combination of any of the listed dosing protocols.

36) The method of claim 35, wherein the daily dose of OCC is selected from any one or more of the following: a) about 140 microg to abut 315 microg of oleandrin per day; b) about 20 microg to about 750 microg; c) about 12 microg to about 300 microg; d) about 12 microg to about 120 microg of oleandrin; e) about 20 microg to about 750 microg; f) about 0.01 microg to about 100 mg; g) about 0.01 microg to about 100 microg; h) about 0.25 to about 50 microg twice daily or about every 12 hours; i) about 0.9 to about 5 microg twice daily or about every 12 hours; j) about 0.5 to about 100 microg/day; k) about 1 to about 80 microg/day; 1) about 1.5 to about 60 microg/day; m) about 1.8 to about 60 microg/day; n) about 1.8 to about 40 microg/day; o) about 0.5 microg/day; p) about 1 microg/day; q) about 1.5 microg/day; r) about 1.8 microg/day; s) about 2 microg/day; or t) about 5 microg/day.

37) (canceled)

38) The method of claim 8, wherein the administration of TMZ and said composition is independently selected upon each occurrence from the group consisting of parenteral, buccal, enteral, intramuscular, subdermal, sublingual, peroral, oral administration, intracranial, intrathecal, intraspinal, or a combination thereof.

39) (canceled)

40) The method of claim 2, wherein the GM is recurrent GM or treatment resistant GM, or wherein the GBM is recurrent GBM or treatment resistant GBM.

41) The method of claim 40, wherein said administering results in reduction in the number and/or size of spheroids of GM or GBM stem cells in the subject.

42) The method of claim 2, wherein the composition or pharmaceutical composition comprises a mixture of oleanolic acid (OA), ursolic acid (UA), and betulinic acid (BA), wherein a) the molar ratio of OA:UA:BA is in the range of about 15.6 OA to about 4 UA to about 1 BA, or about 16 OA to about 4 UA to about 1 BA, or in the range of about 15-16 OA to about 3.5-4.5 UA to about 0.5-1.5 BA, or in the range of about 15.4-15.8 OA to about 3.8-4.2 UA to about 0.8-1.2 BA; b) the molar ratio of the OA:UA is about 4 OA to about 1 UA, the molar ratio OA:UA:BA is about P:Q:1 or greater, wherein P is at least 4, and Q is at least 1, (e.g. about 4:1:1 or greater, about 8:2:1 or greater, or about 16:4:1 or greater), and the molar of OA+UA:BA is about 5:1 or greater (or about 10:1 or greater, or about 20:1 or greater); or c) the molar ratio of UA:BA is about (0.04-0.8):1, the molar ratio of OA:UA:BA is about X:(0.04-0.8):1 or greater, wherein X is about 0.04 or greater.

43) The method of claim 42, wherein the molar ratio of OA:UA:BA is about 4:1:1, about 8:2:1, about 16:4:1, about 32:8:1, about 64:16:1, about 128:32:1, about 256:64:1, about 0.04:0.04:1, about 0.08:0.04:1, about 0.12:0.04.1, about 0.15:0.04:1, about 0.31:0.04:1, about 0.62:0.04:1, about 1.24:0.04:1, about 2.5:0.04:1, about 0.04:0.08:1, about 0.08:0.08:1, about 0.12:0.08.1, about 0.15:0.08:1, about 0.31:0.08:1, about 0.62:0.08:1, about 1.24:0.08:1, about 2.5:0.08:1, or greater.

44) The method of claim 42, wherein the composition a) excludes cardiac glycoside; b) excludes oleandrin; and/or c) comprises the mixture of triterpenes as the primary active ingredient.

45) (canceled)

46) (canceled)

47) (canceled)

48) (canceled)
